SEQUENCE LISTING (78 sequences)

SEQ ID NO: 1 - DNA, 1160 bp, Rhodosporidium toruloides
  CDS (1)..(1104): coding for DAAO
atgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgaattggacgcctttcatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgatggactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtagggcgggatttgtggctgtattgcgggcatctacaagaaaaaaaaaaaaaaaaa

SEQ ID NO: 2 - protein, 368 aa, Rhodosporidium toruloides
MHSQKRVVVLGSGVIGLSSALILARKGYSVHILARDLPEDVSSQTFASPWAGANWTPFMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTMDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

SEQ ID NO: 3 - DNA, 1005 bp, Caenorhabditis elegans
  CDS (1)..(1002): coding for DAAO
atggcaaacataattccgaagattgcaattatcggcgaaggagtcattggatgtacttcagcacttcaaatatcaaaagctataccaaatgcgaaaataactgtgctccacgataaaccatttaaaaaatcgtgcagtgcaggaccagcaggattatttagaatcgattatgaggagaatactgaatacggacgtgcttctttcgcctggttctcacatctctatcgcactacaaaaggatccgaaaccggcgtgaaattagtttctggacatattcaatccgacaacttggagtcattgaagcaacaacaaagagcctatggcgatattgtgtacaactttagattcttggatgatagagaacggctggacatttttcccgaaccatcaaagcactgcattcactacaccgcctacgcatcagaaggtaacaagtacgtgccttatttgaagaatttgctgcttgagcaaaaaatcgagttcaagcaacaagaagtgacgagtttggacgcagtcgccgacgctggttacgatgttattgtaaactgcgcaggcttgtacggtggaaagttggctggtgatgacgatacttgctaccccattagaggagtcattttggaagttgatgcaccatggcacaagcacttcaattatcgagactttactactttcacaattccaaaagagcacagcgtggtggttgggtccaccaagcaggacaatcgatgggatttggagatcaccgacgaggatagaaatgatattttgaaacgatacattgctttacatcctggaatgagagagccaaagattatcaaagaatggtcagcacttcgcccgggacgtaagcatgtcagaattgaagcgcagaagaggacatctgttggaaactcaaaagattatatggttgtgcatcactatggtcacgggagcaacggattcacgttgggttggggaacagcaattgaagcaactaaacttgttaagactgcactaggattataa

SEQ ID NO: 4 - protein, 334 aa, Caenorhabditis elegans
MANIIPKIAIIGEGVIGCTSALQISKAIPNAKITVLHDKPFKKSCSAGPAGLFRIDYEENTEYGRASFAWFSHLYRTTKGSETGVKLVSGHIQSDNLESLKQQQRAYGDIVYNFRFLDDRERLDIFPEPSKHCIHYTAYASEGNKYVPYLKNLLLEQKIEFKQQEVTSLDAVADAGYDVIVNCAGLYGGKLAGDDDTCYPIRGVILEVDAPWHKHFNYRDFTTFTIPKEHSVVVGSTKQDNRWDLEITDEDRNDILKRYIALHPGMREPKIIKEWSALRPGRKHVRIEAQKRTSVGNSKDYMVVHHYGHGSNGFTLGWGTAIEATKLVKTALGL

SEQ ID NO: 5 - DNA, 1186 bp, Nectria haematococca
  CDS (42)..(1124): coding for DAAO
agcgacttgaatttagcgaaaagaacttgtcaaccacaatcatgtccaacacaatcgtcgtcgttggtgccggtgtcattggcttgacgtcggccttgttgctctccaagaacaagggcaacaagatcaccgtcgtggccaagcacatgcccggcgactatgacgttgaatacgcctcgccttttgctggtgccaaccactcccccatggcgacggaagagagcagcgaatgggaacgtcgcacttggtacgagtttaagagactggtcgaggaggtccctgaggccggtgttcatttccagaagtctcgcatccagaggcgcaatgtggacactgaaaaggcgcagaggtctggtttcccagacgccctcttctcgaaagaaccctggttcaagaacatgtttgaggacttccgtgagcagcaccctagcgaggtcatccccggttacgactctggctgcgagttcacatcggtgtgcatcaacacggccatctacctcccctggctcctcggccagtgcatcaagaatggcgtcatcgtcaagcgcgccatcctcaacgacattagcgaggccaagaagctgagccacgcgggcaagacgcccaatatcatcgtcaacgccacgggtctcggctcctacaagctgggcggtgtcgaggacaagaccatggcgcctgcgcggggacagattgtggttgtgcgcaacgagagcagccccatgctcctcacttcaggtgtcgaggacggcggtgctgatgtcatgtacttgatgcagcgagcagctggcggtggcaccatcctgggcggtacctacgacgttggcaactgggagtctcagccagaccccaacatcgcgaatcgcatcatgcagcgcatcgtcgaggtgcggcccgagattgccaacggcaagggcgtcaaggggctgagcgtgatccgacacgccgtcggcatgcggccgtggcgaaaggacggagtcaggatcgaggaggagaagctggatgatgagacttggatcgtgcacaactacggacactctggatggggttaccagggttcgtatggttgtgctgagaatgtagtccagttggttgacaaggtcggcaaggcggccaagtctaagctgtagttgaaaaggcctgaatgagtaatagtaattggatattggaaataccgtatttgccctcg

SEQ ID NO: 6 - protein, 361 aa, Nectria haematococca
MSNTIVVVGAGVIGLTSALLLSKNKGNKITVVAKHMPGDYDVEYASPFAGANHSPMATEESSEWERRTWYEFKRLVEEVPEAGVHFQKSRIQRRNVDTEKAQRSGFPDALFSKEPWFKNMFEDFREQHPSEVIPGYDSGCEFTSVCINTAIYLPWLLGQCIKNGVIVKRAILNDISEAKKLSHAGKTPNIIVNATGLGSYKLGGVEDKTMAPARGQIVVVRNESSPMLLTSGVEDGGADVMYLMQRAAGGGTILGGTYDVGNWESQPDPNIANRIMQRIVEVRPEIANGKGVKGLSVIRHAVGMRPWRKDGVRIEEEKLDDETWIVHNYGHSGWGYQGSYGCAENVVQLVDKVGKAAKSKL

SEQ ID NO: 7 - DNA, 1071 bp, Trigonopsis variabilis
  CDS (1)..(1068): coding for DAAO
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcagccaacgctcccatgttctcaagcgtgatcttcctaaactggaagttgccatgtcggccatctgtcaacgcaatccctggttcaaaaacacagtcgattctttcgagattatcgaggacaggtccaggattgtccacgatgatgtggcttatctagtcgaatttcgttccgtttgtatccacaccggagtctacttgaactggctgatgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagatgtaccctattcgaggacaagtcgtccttgttcgaaactctcttccttttatggcctccttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccaatcctcttacggcatggctgatgaagctgtttcttacgtcgaaagagctcttactcgtccaaacctttag

SEQ ID NO: 8 - protein, 356 aa, Trigonopsis variabilis
MAKIVVIGAGVAGLTTALQLLRKGHEVTIVSEFTPGDLSIGYTSPWAGANWLTFYDGGKLADYDAVSYPILRELARSSPEAGIRLISQRSHVLKRDLPKLEVAMSAICQRNPWFKNTVDSFEIIEDRSRIVHDDVAYLVEFRSVCIHTGVYLNWLMSQCLSLGATVVKRRVNHIKDANLLHSSGSRPDVIVNCSGLFARFLGGVEDKKMYPIRGQVVLVRNSLPFMASFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 9 - DNA, 1047 bp, Schizosaccharomyces pombe
  CDS (22)..(1041): coding for DAAO
atgactaaggaaaataagccaagagatattgtcatcgttggcgctggcgttattggattgaccactgcttggattctttcagacttgggtcttgctcctcgtattaaggtgattgccaagtatacgcctgaagatcgttctgtagaatacacttccccttgggctggcgcaaatttctgtagcatttctgctactgatgacaatgctttgcgctgggataaaatcacttaccatcgtttcgcctacttggcgaaaactcgtcctgaagcaggaatccgttttgctgatcttcgagaattgtgggagtacgagccgaaacacgacaaaatcagatcctggaatacctatgtcagagatttcaaagttatccctgaaaaagatcttccaggagaatgtatctacggacataaggccaccacctttttaatcaacgctcctcattacttgaattatatgtacaagctgctcattgaagctggcgtcgaatttgaaaagaaagaattgagtcacatcaaagagactgtcgaagaaactccagaagcttcagtagtatttaattgcactggtctctgggcttccaaattgggtggcgttgaagacccggacgtttatccgactcgtggacatgttgttttggttaaggctcctcatgtaacagaaactcgcattttgaatggcaagaactctgatacctatattattcctcgtcccttaaatggtggagtcatttgcggcggtttcatgcaaccaggaaactgggatcgtgaaattcaccctgaagacactttggatatccttaagagaacatcggctttgatgccagaattgttccacggcaagggtccggagggtgctgaaattattcaagaatgtgtcggattccgtccttctcgaaagggtggtgcccgcgtagagcttgatgttgttcccggcacctcagtcccccttgttcatgattacggtgcttctggcacaggataccaagctggttatggtatggctcttgactctgtcatgttggctcttcctaaaatcaaattggcttag

SEQ ID NO: 10 - protein, 340 aa, Schizosaccharomyces pombe
RDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTYIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKL

SEQ ID NO: 11 - DNA, 963 bp, Streptomyces coelicolor
  CDS (31)..(957): coding for DAAO
gtggaaaccgaactggatgacgagcgggatggcgaagtcgtcgtggtcggcggcggggtgatcgggctgacgacggccgtcgtcctcgccgagcggggcagacgggtgcggctgtggacccgggagcccgcggagcggaccacctcggtggtagcgggcgggctgtggtggccgtaccggatcgagccggtcgcgctggcccaggcctgggcgctgcgttccctggacgtgtacgaggagctggcggcacggcccgggcagaccggcgtacgcatgctcgaaggggtgctcggcgagaccggcctggacgaggtggacgggtgggccgcggcccggctgccggggctgcgcgcggcgagcgccgccgagtacgccgggacggggctgtgggcgcggctgccgctcatcgacatgtcgacccatctgccgtggctgcgggagcggctgctggccgcgggcggcacggtggaggaccgcgcggtgaccgatctggccgaggcggacgcgccggtggtggtcaactgcaccggcctgggcgcccgggagctggtgccggacccggcggtacggccggtgcgcggacagctggtcgtcgtggagaaccccggcatccacaactggctggtcgcggccgacgcggactccggggagacgacgtacttccttccgcagccgggacggctcctgctgggcggcacggctgaggaggacgcctggtcgaccgagccggacccggaggtcgcggcggccatcgtgcgacggtgcgcggccctgcgtcccgagatcgccggagcgcgggtgctcgcgcacctggtggggctgcggccggcccgggacgcggtccggctggagcgcgggacgctgccggacgggcgccggctggtgcacaactacggtcacggcggcgcgggcgtcaccgtggcctggggctgcgctcaggaggcggcccggctcgcctcctga

SEQ ID NO: 12 - protein, 309 aa, Streptomyces coelicolor
  misc_feature (880)..(936): DAAO signature
GEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTHLPWLRERLLAAGGTVEDRAVTDLAEADAPVVVNCTGLGARELVPDPAVRPVRGQLVVVENPGIHNWLVAADADSGETTYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLA

SEQ ID NO: 13 - DNA, 1038 bp, Candida boidinii
  CDS (1)..(1035): coding for DAAO
atgggtgatcaaattgttgttcttggttccggtattattggtttatatactacatactgtttaatctatgaggctggatgtgctccagctaaaattactattgttgctgaatttttaccaggtgatcaatctacattatatacatctccatgggcaggtggtaatttttcttgtatttcaccagctgatgatacaacattggcttatgataaattcacatatcttaatttattcaagattcacaaaaaattaggtggaccagaatgtggattagataataagccaagtactgaatattgggatttttatcctggtgatgaaaaagtcaattctttaaaacaatatcttaaagattttaaagttattccaaaatcagaattaccagaaggtgttgaatatggtattagttatactacatggaatttcaactgtcctgttttcttacaaaatatggctaattttttaaataaaagaaatgttaccattattagaaaacatttaacacatatttctcaagcttatttaacagttaatacaaaagttgttttcaactgtacaggtattggtgctgctgatttaggtggtgttaaagatgaaaaagtttatccaactagaggacaagttgttgttgttagagctccacatattcaagaaaataaaatgagatggggtaaagactatgctacttatattattccaagaccatattctaatggtgaattagtcttaggtggtttcttacaaaaggataattggacaggtaatacttttggttttgaaactgatgatattgttagtagaactacatctttattaccaaagattttagatgaaccacttcatattattagagttgcagctggtttaagaccaagtagacatggtggtccaagaattgaagctgaagtttgtgaagaaggtaaattaactattcataattatggtgcttctggatatggttatcaagctggttatggtatgtcttatgaagctgtcaaacttttagttgataaccaaaaagttaaagctaaactttag

SEQ ID NO: 14 - protein, 345 aa, Candida boidinii
MGDQIVVLGSGIIGLYTTYCLIYEAGCAPAKITIVAEFLPGDQSTLYTSPWAGGNFSCISPADDTTLAYDKFTYLNLFKIHKKLGGPECGLDNKPSTEYWDFYPGDEKVNSLKQYLKDFKVIPKSELPEGVEYGISYTTWNFNCPVFLQNMANFLNKRNVTIIRKHLTHISQAYLTVNTKVVFNCTGIGAADLGGVKDEKVYPTRGQVVVVRAPHIQENKMRWGKDYATYIIPRPYSNGELVLGGFLQKDNWTGNTFGFETDDIVSRTTSLLPKILDEPLHIIRVAAGLRPSRHGGPRIEAEVCEEGKLTIHNYGASGYGYQAGYGMSYEAVKLLVDNQKVKAKL

SEQ ID NO: 15 - DNA, 12466 bp, Unknown
  Nucleic acid sequence for vector daaoSceITetON
aatattcaaacaaacacatacagcgcgacttatcatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccccaaactgtctcacgacgttttgaacccagattaccctgttatccctagtcgagcggccgccagtgtgatggatatctgcagaattcgcccttttagatcttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatatggtcgactaaagggcgaattccagcacactggcggccgttactagcccgggctcgagcaaatgtctagaaaggccttatatacgtaaagggtcttgcgaagactagatcactctatctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatccgtgatagagaaaagtgaaagtcgagtttaccactccctatcagtgatagagaaaagtgaaagtcgagtttaccactccctatcagtgatagagaaaatgaaagtcgagtttaccactccctatcagtgatagagaaaagtgaaagtcgagctcggtaccgagctcgaattcagcacactggcggccgttactagtggatcaattcactggccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgcctacaacttcgactcccgcgccgcgccgtggtaccgctggaacgcctcgtcgacgagctgcgcgacatcctccgccgcgccccaactctgctggtatcccgcactcgagaagccatacgcatgcacaagcgtgacctccttctccttcgccgctcgtgcgctgcccctgccgagcgagaggggcgactttgtccggtcgagaggcaggacgatccgttctgcctcaacgcggggtccgcctcgtcgtgcaggtcgcaagccgacgttgtggcggaggacctcgatgccttcgatcgttccgtcgctcgagatggtcgggtcgaggcgcaagcagtgcttgaggatccgctggaccgtctctgggttgacagacaagtcccagtctcccacgccgtacgtcccgccgcagatgacttcgccacctggtcggggaatgatgtaggcgggagaagcggggtcggacgagtccatcgtgcatcgcttgcatggggacttgacgaggacggtttgcccgcggattggctcggcggcttggtcgtcgatgcccgcaatcgacttggcgccaagtcccgtagcgttgaccaccaaatccgcaccgtcgaacgcctgctcaagcgacgtaacggtccgtctctcaaacgtcgcgccgagcttctgcagctctcttgcaaggtactggcagtactttggtgcgtggacggagagggtgtcgtaggttacgccgatagcgccaggtggacattcggaagatgggagggggcggtaatttggcgtgatgtccttgtaccagtgcccgagcaagccgtcttcgttctgcgcgaaccgcctcgtccccttgagccacatggcatggcccgtcgggaccaactcgacccacttcttgaaagtcgattcttcccattttgcttgtcgaggaccgtctgtaagcgtcatgaaaggcgtccaattcgcgccagcccatggtgaagcgaaagtctggctcgagacgtcctccggcaagtcgcgcgcgagaatatgcacgctgtagcccttccgagcgaggatgagggcgctgctcagaccgataacgcctgatccgaggacaacgacgcgcttctgcgagtgcatgggccctcgactagagtcgagatccgatatcgcccgggctcgagtctttgttttttactttggttcatgacactcagagacttgagagaagcaatatatagacttttttttgttttttttttgtggtcacgtttattttcctattggagacggtaacgaagatcgaacctgtggtggaaatgaaacaaggtgggactagcccacgtggtttcttttctctgcattgatttgtttttgttttttttgtaaagttcacatcaaacctactaataattgagaagaaaaataaaatctattgattgattaaaccagccgatgctttatgtctgaatataaaaaagaagtgaaaaccccgtttaagaattacaacggtggtttacaaagtatttggacacaataaatccaaacgaaataaaacaaaatggagaactaccaaataaaaaacaaataaaaaacttaaaagaatttattccattttttttcccgtagaatttattcttttatggattccttaaatccatatttgatgcattttgattcctcataataggtaataatatatactatgttatagatatgtttctaattcgtattaacctacctttttttggtcgtacgattctacctaataatattgaacggaattgatgttttggaccacttagaaagtattttttttttggtttgtcttagctgtatttcattaaatataaatttaaataagaaatgtcataaataaaatttgacgtatagattttttaaatccattttatgttatttaatatttgaaatgtgagtttggctcctatttaatcttaggatgggttaatactaagttttccttaatgaattatctcagagaaactggattaaataaactaaaaaatagatcaatgtgttttggtccggtcaaatatctttggatttactattattggcgaaaagaaagtctcatatagtaaatcatattcctacaagagaaatcaaaatttttgaattaacatggattgtatagtttcttatataaccaattagttcgcatcaagaaaaccaaaccccaattaataatcaaacgggcttggtaggaatatttcattgcagctttcagataaaagaaaaaaacacacactcaagtcttttatttcatctttcttacttgcaggaactcaaattccactttgccacttttctttacaaataaacacaaattgtcaatgaaacgaaatagtctttttatgcaaacactgtttgtcttttttcgatcacgtttctgattgtgacagccatccatatatatagggaatgtaaaacaacaacatgtgaagtcacatatacgtaatggtttagcatagcttctattttcgttgtcaatattagtcattccaaaacatttttaagaaaaataaattaatatatgtatattcttggaactaatgtatgtggaaatacagtaacttaattattaaacattctaaatgcaaatatgcaaagaaaaaaaagaaaagaacacaactgaaatcaaagccagattcataataattggctacatggttgtagaatgtagggtaacacaacatccagaattgaacactcaaattggatgatagatggataatctttagatacaagagaattggttctcttccattattaacgaaaataaagaaaaaaagtttagcataaaagtttgaaactcaacataacattttgaacttgactccttcataggagtgacatgaactgacgaatcacaaccgattacttgtttgagtcatcttccgctttctccaccttcgaaatgaatgtgaccggtttcttcgggtgctcatttacggtcaagtgtaaaacatctggtctcgaggtacctggtagggataacagggtaatctgggttcaaaacgtcgtgagacagtttggtgcaggtcgaaattcgagctcggtaccaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatcttgcgccgggtaccgagctcggtagcaattcccgaggctgtagccgacgatggtgcgccaggagagttgttgatctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataacgcgttctctagtgaaaaaccttgttggcataaaaaggctaattgattttcgagagtttcatactgtttttctgtaggccgtgtatctgaatgtacttttgctccattgcgatgacttagtaaagcacatctaaaacttttagcgttattgcgtaaaaaatcttgccagctttccccttttaaagggcaaaagtgagtatggtgcctatctaacatctcaatggctaaggcgtcgagcaaagcccgcttattttttacatgccaatacagtgtaggctgctctacaccaagcttctgggcgagtttacgggttgttaaaccttcgattccgacctcattaagcagctctaatgcgctgttaatcactttacttttatctaatctagacatggtcgatcgactctagactagtggatccgatatcgcccgggctcgactctagagtttcgaagattttagtgtaatgtgtgtgctcactactatgaagctttgcacttaaaaaaatagaatgagtgatgaggtttatatggtgaaaaaaactatgaaattttgatattttgatatatctttctcgtgagtcatattcacggaccatgttgcagcaaattggaattaaactattcattttttatgttaaatcattgattgatttttagtgggcctcgttacatattcaagagttagaatgaattcaaacaaactaggccagaaaaaaggatgtggggccatttttttgtgtcttaagaatttgtttatttttttcatggataaggggaatcaatggaaaaagtttgatgtactagaggacatttttttaacatgtagtgacaagtagtgctattattcgacccgtgatgaaaggggcaatcttaatctttttttcataaatctgcacatgtgatgctttaattatgctttagactttgtgctaaactattggtaatttctttttgtaatcgaatcaagtatcttttaaactatgtatgaaatgtgtcatcctaaaaacaacattttgctagttttagactttgatgtttatatgcttaatggaagaagcaatatgttgatgtttattgggtaaaagaaagggacttgattgagtatgtaattgacaactatgattttatattggatttgatattcctaacattaatttaagtgtgtgggtttcaaagcatgttatgctagtgattcttgtgtttgatgcttgaaaaatctacattcatccttgaatggagggacaaactttgaatgacttttgaataggtgtaaaatccaatcctccctcagcttcacaaaaaattgcggacggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgattttccgggggatcctctagagtcgaggtaccgagctcgaattcactggccgtcgttttacaacgactcagtactgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagacgccccggtgaatcgtggcaaggggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggcctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatg

SEQ ID NO: 16 - DNA, 12539 bp, Unknown
  Nucleic acid sequence for vector daaoNit-PRecombination
aatattcaaacaaacacatacagcgcgacttatcatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccccaaactgtctcacgacgttttgaacccagattaccctgttatccctagtcgagcggccgccagtgtgatggatatctgcagaattcgcccttttagatcagcacactggcggccgttactagtggatcaattcactggccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgcctacaacttcgactcccgcgccgcgccgtggtaccgctggaacgcctcgtcgacgagctgcgcgacatcctccgccgcgccccaactctgctggtatcccgcactcgagaagccatacgcatgcacaagcgtgacctccttctccttcgccgctcgtgcgctgcccctgccgagcgagaggggcgactttgtccggtcgagaggcaggacgatccgttctgcctcaacgcggggtccgcctcgtcgtgcaggtcgcaagccgacgttgtggcggaggacctcgatgccttcgatcgttccgtcgctcgagatggtcgggtcgaggcgcaagcagtgcttgaggatccgctggaccgtctctgggttgacagacaagtcccagtctcccacgccgtacgtcccgccgcagatgacttcgccacctggtcggggaatgatgtaggcgggagaagcggggtcggacgagtccatcgtgcatcgcttgcatggggacttgacgaggacggtttgcccgcggattggctcggcggcttggtcgtcgatgcccgcaatcgacttggcgccaagtcccgtagcgttgaccaccaaatccgcaccgtcgaacgcctgctcaagcgacgtaacggtccgtctctcaaacgtcgcgccgagcttctgcagctctcttgcaaggtactggcagtactttggtgcgtggacggagagggtgtcgtaggttacgccgatagcgccaggtggacattcggaagatgggagggggcggtaatttggcgtgatgtccttgtaccagtgcccgagcaagccgtcttcgttctgcgcgaaccgcctcgtccccttgagccacatggcatggcccgtcgggaccaactcgacccacttcttgaaagtcgattcttcccattttgcttgtcgaggaccgtctgtaagcgtcatgaaaggcgtccaattcgcgccagcccatggtgaagcgaaagtctggctcgagacgtcctccggcaagtcgcgcgcgagaatatgcacgctgtagcccttccgagcgaggatgagggcgctgctcagaccgataacgcctgatccgaggacaacgacgcgcttctgcgagtgcatgggccctcgactagagtcgagatccgatatcgcccgggctcgagtctttgttttttactttggttcatgacactcagagacttgagagaagcaatatatagacttttttttgttttttttttgtggtcacgtttattttcctattggagacggtaacgaagatcgaacctgtggtggaaatgaaacaaggtgggactagcccacgtggtttcttttctctgcattgatttgtttttgttttttttgtaaagttcacatcaaacctactaataattgagaagaaaaataaaatctattgattgattaaaccagccgatgctttatgtctgaatataaaaaagaagtgaaaaccccgtttaagaattacaacggtggtttacaaagtatttggacacaataaatccaaacgaaataaaacaaaatggagaactaccaaataaaaaacaaataaaaaacttaaaagaatttattccattttttttcccgtagaatttattcttttatggattccttaaatccatatttgatgcattttgattcctcataataggtaataatatatactatgttatagatatgtttctaattcgtattaacctacctttttttggtcgtacgattctacctaataatattgaacggaattgatgttttggaccacttagaaagtattttttttttggtttgtcttagctgtatttcattaaatataaatttaaataagaaatgtcataaataaaatttgacgtatagattttttaaatccattttatgttatttaatatttgaaatgtgagtttggctcctatttaatcttaggatgggttaatactaagttttccttaatgaattatctcagagaaactggattaaataaactaaaaaatagatcaatgtgttttggtccggtcaaatatctttggatttactattattggcgaaaagaaagtctcatatagtaaatcatattcctacaagagaaatcaaaatttttgaattaacatggattgtatagtttcttatataaccaattagttcgcatcaagaaaaccaaaccccaattaataatcaaacgggcttggtaggaatatttcattgcagctttcagataaaagaaaaaaacacacactcaagtcttttatttcatctttcttacttgcaggaactcaaattccactttgccacttttctttacaaataaacacaaattgtcaatgaaacgaaatagtctttttatgcaaacactgtttgtcttttttcgatcacgtttctgattgtgacagccatccatatatatagggaatgtaaaacaacaacatgtgaagtcacatatacgtaatggtttagcatagcttctattttcgttgtcaatattagtcattccaaaacatttttaagaaaaataaattaatatatgtatattcttggaactaatgtatgtggaaatacagtaacttaattattaaacattctaaatgcaaatatgcaaagaaaaaaaagaaaagaacacaactgaaatcaaagccagattcataataattggctacatggttgtagaatgtagggtaacacaacatccagaattgaacactcaaattggatgatagatggataatctttagatacaagagaattggttctcttccattattaacgaaaataaagaaaaaaagtttagcataaaagtttgaaactcaacataacattttgaacttgactccttcataggagtgacatgaactgacgaatcacaaccgattacttgtttgagtcatcttccgctttctccaccttcgaaatgaatgtgaccggtttcttcgggtgctcatttacggtcaagtgtaaaacatctggtctcgaggtacctggtagggataacagggtaatctgggttcaaaacgtcgtgagacagtttggtgcaggtcgaaattcgagctcggtacccggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgccgggtaccgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgaaatattcccgtgcacttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgaccaattccacagttttcgcgatccagactgaatgcccacaggccgtcgagttttttgatttcacgggttggggtttctacaggacgtaccatggtcgatcgactctagactagtggatccgatatcgcccgggctcgactctagatgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcaggatccgggggatcctctagagtcgaggtaccgagctcgaattcactggccgtcgttttacaacgactcagtactgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagacgccccggtgaatcgtggcaaggggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggcctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatg

SEQ ID NO: 17 - protein, 19 aa, Unknown
  Consensus motif sequence of D-amino acid oxidase
XXHXYGXXXXGXXXXXGXA

SEQ ID NO: 18 - DNA, 85 bp, Unknown
  Preferred recombination site for suitable recombinases
aactctcatcgcttcggataacttcctgttatccgaaacatatcactcactttggtgatttcaccgtaactgtctatgattaatg

SEQ ID NO: 19 - DNA, 48 bp, Unknown
  Preferred recombination site for suitable recombinases
gaagttcctattccgaagttcctattctctagaaagtataggaacttc

SEQ ID NO: 20 - DNA, 58 bp, Unknown
  Preferred recombination site for suitable recombinases
cgagatcatatcactgtggacgttgatgaaagaatacgttattctttcatcaaatcgt

SEQ ID NO: 21 - DNA, 23 bp, Unknown
  DNA-endonuclease recognition sequence
ctagatgaaataacataaggtgg

SEQ ID NO: 22 - DNA, 39 bp, Unknown
  DNA-endonuclease recognition sequence
ttgaggaggtttctctgtaaataannnnnnnnnnnnnnn

SEQ ID NO: 23 - DNA, 24 bp, Unknown
  DNA-endonuclease recognition sequence
ttttttggtcatccagaagtatat

SEQ ID NO: 24 - DNA, 30 bp, Unknown
  DNA-endonuclease recognition sequence
ctgggttcaaaacgtcgtgagacagtttgg

SEQ ID NO: 25 - DNA, 30 bp, Unknown
  DNA-endonuclease recognition sequence
gtactagcatggggtcaaatgtctttctgg

SEQ ID NO: 26 - DNA, 18 bp, Unknown
  DNA-endonuclease recognition sequence
tcgtagcagctcacggtt

SEQ ID NO: 27 - DNA, 30 bp, Unknown; identical to SEQ ID NO: 24
  DNA-endonuclease recognition sequence

SEQ ID NO: 28 - DNA, 30 bp, Unknown
  DNA-endonuclease recognition sequence
gaaggtttggcacctcgatgtcggctcatc

SEQ ID NO: 29 - DNA, 23 bp, Unknown
  DNA-endonuclease recognition sequence
cgatcctaaggtagcgaaattca

SEQ ID NO: 30 - DNA, 20 bp, Unknown
  DNA-endonuclease recognition sequence
cccggctaactctgtgccag

SEQ ID NO: 31 - DNA, 29 bp, Unknown
  DNA-endonuclease recognition sequence
cgtaactataacggtcctaaggtagcgaa

SEQ ID NO: 32 - DNA, 30 bp, Unknown
  DNA-endonuclease recognition sequence
atgccttgccgggtaagttccggcgcgcat

SEQ ID NO: 33 - DNA, 30 bp, Unknown
  DNA-endonuclease recognition sequence
agttacgctagggataacagggtaatatag

SEQ ID NO: 34 - DNA, 18 bp, Unknown
  DNA-endonuclease recognition sequence
tagggataacagggtaat

SEQ ID NO: 35 - DNA, 29 bp, Unknown
  DNA-endonuclease recognition sequence
ttttgattctttggtcaccctgaagtata

SEQ ID NO: 36 - DNA, 29 bp, Unknown
  DNA-endonuclease recognition sequence
attggaggttttggtaactatttattacc

SEQ ID NO: 37 - DNA, 29 bp, Unknown
  DNA-endonuclease recognition sequence
tcttttctcttgattagccctaatctacg

SEQ ID NO: 38 - DNA, 24 bp, Unknown
  DNA-endonuclease recognition sequence
aataattttcttcttagtaatgcc

SEQ ID NO: 39 - DNA, 24 bp, Unknown
  DNA-endonuclease recognition sequence
gttatttaatgttttagtagttgg

SEQ ID NO: 40 - DNA, 28 bp, Unknown
  DNA-endonuclease recognition sequence
tgtcacattgaggtgcactagttattac

SEQ ID NO: 41 - DNA, 30 bp, Unknown
  DNA-endonuclease recognition sequence
atctatgtcgggtgcggagaaagaggtaat

SEQ ID NO: 42 - DNA, 24 bp, Unknown
  DNA-endonuclease recognition sequence
gatgctgtaggcataggcttggtt

SEQ ID NO: 43 - DNA, 20 bp, Unknown
  DNA-endonuclease recognition sequence
ctttccgcaacagtaaaatt

SEQ ID NO: 44 - DNA, 24 bp, Unknown
  DNA-endonuclease recognition sequence
agtaatgagcctaacgctcagcaa

SEQ ID NO: 45 - DNA, 63 bp, Unknown
  DNA-endonuclease recognition sequence
agtaatgagcctaacgctcaacaannnnnnnnnnnnnnnnnnnnnnnnnnnnnnnnnnnnnnn

SEQ ID NO: 46 - DNA, 24 bp, Unknown
  DNA-endonuclease recognition sequence
cacatccataaccatatcattttt

SEQ ID NO: 47 - DNA, 30 bp, Unknown; identical to SEQ ID NO: 24
  DNA-endonuclease recognition sequence

SEQ ID NO: 48 - DNA, 21 bp, Unknown
  DNA-endonuclease recognition sequence
aagtctggtgccagcacccgc

SEQ ID NO: 49 - DNA, 21 bp, Unknown
  DNA-endonuclease recognition sequence
aagtctggtgccagcacccgc

SEQ ID NO: 50 - DNA, 21 bp, Unknown
  DNA-endonuclease recognition sequence
aagtctggtgccagcacccgc

SEQ ID NO: 51 - DNA, 30 bp, Unknown; identical to SEQ ID NO: 24
  DNA-endonuclease recognition sequence

SEQ ID NO: 52 - DNA, 25 bp, Unknown
  DNA-endonuclease recognition sequence
gcgagcccgtaagggtgtgtacggg

SEQ ID NO: 53 - DNA, 29 bp, Unknown
  DNA-endonuclease recognition sequence
taactatgactctcttaaggtagccaaat

SEQ ID NO: 54 - DNA, 28 bp, Unknown
  DNA-endonuclease recognition sequence
tgtcacattgaggtgcactagttattac

SEQ ID NO: 55 - DNA, 19 bp, Unknown
  DNA-endonuclease recognition sequence
gtcgggctcataacccgaa

SEQ ID NO: 56 - DNA, 30 bp, Unknown
  DNA-endonuclease recognition sequence
gaagatgggaggagggaccggactcaactt

SEQ ID NO: 57 - DNA, 29 bp, Unknown
  DNA-endonuclease recognition sequence
acgaatccatgtggagaagagcctctata

SEQ ID NO: 58 - DNA, 19 bp, Unknown
  DNA-endonuclease recognition sequence
gattttagatccctgtacc

SEQ ID NO: 59 - DNA, 15 bp, Unknown
  DNA-endonuclease recognition sequence
cagtactacggttac

SEQ ID NO: 60 - DNA, 30 bp, Unknown
  DNA-endonuclease recognition sequence
aaaatcctggcaaacagctattatgggtat

SEQ ID NO: 61 - DNA, 26 bp, Unknown
  DNA-endonuclease recognition sequence
tagattttaggtcgctatatccttcc

SEQ ID NO: 62 - DNA, 22 bp, Unknown
  DNA-endonuclease recognition sequence
taygcngayacngacggyttyt

SEQ ID NO: 63 - DNA, 30 bp, Unknown
  DNA-endonuclease recognition sequence
aaattgcttgcaaacagctattacggctat

SEQ ID NO: 64 - DNA, 24 bp, Unknown
  DNA-endonuclease recognition sequence
agtggtatcaacgctcagtagatg

SEQ ID NO: 65 - DNA, 30 bp, Unknown
  DNA-endonuclease recognition sequence
gcttatgagtatgaagtgaacacgttattc

SEQ ID NO: 66 - DNA, 38 bp, Unknown
  DNA-endonuclease recognition sequence
gaaacacaagaaatgtttagtaaannnnnnnnnnnnnn

SEQ ID NO: 67 - DNA, 29 bp, Unknown
  DNA-endonuclease recognition sequence
tttaatcctcgcttcagatatggcaactg

SEQ ID NO: 68 - DNA, 24 bp, Unknown
  DNA-endonuclease recognition sequence
caaaacgtcgtaagttccggcgcg

SEQ ID NO: 69 - DNA, 24 bp, Unknown
  DNA-endonuclease recognition sequence
agtaatgagcctaacgctcagcaa

SEQ ID NO: 70 - DNA, 24 bp, Unknown
  DNA-endonuclease recognition sequence
gttaggctcaagctgagcattact

SEQ ID NO: 71 - DNA, 28 bp, Unknown
  DNA-endonuclease recognition sequence
gagtaagagcccgtagtaatgacatggc

SEQ ID NO: 72 - DNA, 19 bp, Unknown
  DNA-endonuclease recognition sequence
cttcagtatgccccgaaac

SEQ ID NO: 73 - DNA, 20 bp, Unknown
  DNA-endonuclease recognition sequence
tcttgcacctacacaatcca

SEQ ID NO: 74 - DNA, 22 bp, Unknown
  DNA-endonuclease recognition sequence
cgtagctgcccagtatgagtca

SEQ ID NO: 75 - DNA, 22 bp, Unknown
  DNA-endonuclease recognition sequence
gggggcagccagtggtcccgtt

SEQ ID NO: 76 - DNA, 23 bp, Unknown
  DNA-endonuclease recognition sequence
acccctgtggagaggagcccctc

SEQ ID NO: 77 - DNA, 30 bp, Unknown
  PCR primer
attagatcttactactcgaaggacgccatg

SEQ ID NO: 78 - DNA, 30 bp, Unknown
  PCR primer
attagatctacagccacaattcccgcccta

(19) United States
(12) Patent Application Publication
(10) Pub. No.: US 2008/0050819 A1
(43) Pub. Date: Feb. 28, 2008

(54) Constructs for Marker Excision Based on Dual-Function Selection Marker

(75) Inventors: Helke Hillebrand, Mannheim (DE); Marcus Ebneth, Berlin (DE); Torgny Nasholm, Holmsund (SE); Oskar Erikson, Holmsund (SE); Magnus Hertzberg, Umea (SE)

Correspondence Address: CONNOLLY BOVE LODGE & HUTZ, LLP, P O BOX 2207, WILMINGTON, DE, 19899, US

(73) Assignees: BASF Plant Science GmbH, Ludwigshafen (DE); SweTree Technologies AB, Umea (SE)

(21) Appl. No.: 10/593,181

(22) PCT Filed: Mar. 15, 2005
(86) PCT No.: PCT/EP05/02734

§ 371 Date: Sep. 15, 2006

(30) Foreign Application Priority Data
Mar. 17, 2004 (EP) 04006358.8

(51) Int. Cl. A01H 5/00; C07H 21/04; C12N 15/00; C12N 5/04

(52) U.S. Cl. 435/419; 435/320.1; 536/23.1; 800/276; 800/278

(57) ABSTRACT

The invention relates to improved construct and methods for eliminating maker sequences from the genome of plants, based on dual-function selection marker which—depending on the employed compound—can act as both negative and counter-selection marker.

### FIELD OF THE INVENTION

The invention relates to improved construct and methods for eliminating maker sequences from the genome of plants, based on dual-function selection marker which—depending on the employed compound—can act as both negative and counter-selection marker.

### BACKGROUND OF THE INVENTION

An aim of plant biotechnology is the generation of plants with advantageous novel characteristics, for example for increasing agricultural productivity, improving the quality in foodstuffs or for the production of certain chemicals or pharmaceuticals (Dunwell J M (2000) J Exp Bot 51:487-96).

There are various methods described in art for inserting heterogenous DNA sequences into the genome of a host cell or organism. Because of the low insertion-frequency, it is generally required to employ selection marker to select for cells or organisms which have successfully incorporated the transgenic construct. Selectable markers enable transgenic cells or organisms (e.g., plants or plant cells) to be identified after transformation. They can be divided into positive selection marker (conferring a selective advantage), negative selection marker (compensating a selection disadvantage), and counter-selection marker (conferring a selection disadvantage), respectively.

Negative selection markers are most often employed in methods for producing transgenic cells or organisms. Such negative selection markers confer for example a resistance to a biocidal compound such as a metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Examples—especially suitable for plant transformation—are:
Phosphinothricin acetyltransferases (PAT; also named Bialophos® resistance; bar; de Block 1987; EP 0 333 033; U.S. Pat. No. 4,975,374)
5-enolpyruvylshikimate-3-phosphate synthase (EPSPS) conferring resistance to Glyphosate® (N-(phosphonomethyl)glycine) (Shah 1986)
Glyphosate® degrading enzymes (Glyphosate® oxidoreductase; gox),
Dalapon® inactivating dehalogenases (deh)
sulfonylurea- and imidazolinone-inactivating acetolactate synthases (for example mutated ALS variants with, for example, the S4 and/or Hra mutation
Bromoxynil® degrading nitrilases (bxn)
Kanamycin- or. G418-resistance genes (NPTII; NPTI) coding e.g., for neomycin phosphotransferases (Fraley 1983)
2-Desoxyglucose-6-phosphate phosphatase (DOG^{R}1-Gene product; WO 98/45456; EP 0 807 836) conferring resistance against 2-desoxyglucose (Randez-Gil 1995).
hygromycin phosphotransferase (HPT), which mediates resistance to hygromycin (Vanden Elzen 1985).
dihydrofolate reductase (Eichholtz 1987)
D-amino acid metabolizing enzyme (e.g., D-amino acid dehydratases or oxidases; WO 03/060133)

Additional negative selectable marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Hayford 1988; Jones 1987; Svab 1990; Hille 1986).

Additional selection markers are those which do not result in detoxification of a biocidal compound but confer an advantage by increased or improved regeneration, growth, propagation, multiplication as the like of the cell or organism comprising such kind of “positive selection marker”. Examples are isopentenyltransferase (a key enzyme of the cytokinin biosynthesis facilitating regeneration of transformed plant cells by selection on cytokinin-free medium; Ebinuma 2000a; Ebinuma 2000b). Additional positive selection markers, which confer a growth advantage to a transformed plant cells in comparison with a non-transformed one, are described e.g., in EP-A 0 601 092. Growth stimulation selection markers may include (but shall not be limited to) β-Glucuronidase (in combination with e.g., a cytokinin glucuronide), mannose-6-phosphate isomerase (in combination with mannose), UDP-galactose-4-epimerase (in combination with e.g., galactose).

The selectable marker gene is useful during the transformation process to select for, and identify, transformed organisms, but typically provides no useful function once the transformed organism has been identified and contributes substantially to the lack of acceptance of these “gene food” products among consumers (Kuiper H A et al. (2001) Plant J. 27, 503-528), and few markers are available that are not based on these mechanisms (Hare P & Chua N H (2002) Nat. Biotechnol. 20, 575-580). Thus, there is a demand for new markers for both research and commercial crop production. Alternatively, there are multiple attempts to develop techniques by means of which marker DNA can be excised from plant genome (Ow D W and Medberry S L (1995) Crit. Rev in Plant Sci 14:239-261; Gleave A P et al. (1999) Plant Mol. Biol. 40, 223-23).

The person skilled in the art is familiar with a variety of systems for the site-directed removal of recombinantly introduced nucleic acid sequences. They are mainly based on the use of sequence specific recombinases. Various sequence-specific recombination systems are described, such as the Cre/lox system of the bacteriophage P1 (Dale E C and Ow D W (1991) Proc Natl Acad Sci USA 88:10558-10562; Russell S H et al. (1992) Mol Gene Genet 234: 49-59; Osborne B I et al. (1995) Plant J. 7, 687-701), the yeast FLP/FRT system (Kilby N J et al. (1995) Plant J 8:637-652; Lyznik L A et al. (1996) Nucleic Acids Res 24:3784-3789), the Mu phage Gin recombinase, the E. coli Pin recombinase or the R/RS system of the plasmid pSR1 (Onouchi H et al. (1995) Mol Gen Genet 247:653-660; Sugita Ket al. (2000) Plant J. 22:461-469).

Zubko et al. (Nature Biotech (April 2000) 18(4):442-445) describe a system for the deletion of nucleic acid sequences, where the sequence to be deleted is flanked by two 352 bp attP recognition sequences from the bacteriophage Lambda. Deletion of the flanked region takes place independently of the expression of helper proteins in two out of eleven transgenic tobacco lines by spontaneous intrachromosomal recombination between the attP recognition regions.

WO 02/29071 discloses a method for conditional excision of transgenic sequences from the genome of a transgenic organism. Excision occurs directly by action of an enzyme (e.g., a recombinase or a endonuclease). Self-excising constructs based on a site-specific recombinase are described in WO97/037012 and WO02/10415.

WO 03/004659 describes a recombination system based on homologous recombination between two homologous sequences induced by action of a sequence specific double-strand break inducing enzyme, preferably a meganuclease (homing-endonuclease).

Since also the marker excision efficiency is most often low, the systems are most often combined with counter-selection marker. These are sequences encoding for enzymes which are able to convert a non-toxic compound into a toxic compound. In consequence, only cells will survive treatment with said non-toxic compound which are lacking said counter-selection marker, thereby allowing for selection of cells which have successfully undergone sequence (e.g., marker) deletion. Typical counter-selection markers known in the art are for example
a) cytosine deaminases (CodA) in combination with 5-fluorocytosine (5-FC) (WO 93/01281; U.S. Pat. No. 5,358,866; Gleave A P et al. (1999) Plant Mol Biol 40(2):223-35; Perera R J et al. (1993) Plant Mol Biol 23(4):793-799; Stougaard J (1993) Plant J 3:755-761); EP-A1 595 837; Mullen C A et al. (1992) Proc Natl Acad Sci USA 89(1):33-37; Kobayashi T et al. (1995) Jpn J Genet 70(3):409-422; Schlaman H R M & Hooykaas P F F (1997) Plant J 11:1377-1385; Xiaohui Wang H et al. (2001) Gene 272(1-2): 249-255; Koprek T et al. (1999) Plant J 19(6):719-726; Gleave A P et al. (1999) Plant Mol Biol 40(2):223-235; Gallego M E (1999) Plant Mol Biol 39(1):83-93; Salomon S & Puchta H (1998) EMBO J. 17(20):6086-6095; Thykjaer T et al. (1997) Plant Mol Biol 35(4):523-530; Serino G (1997) Plant J 12(3):697-701; Risseeuw E (1997) Plant J 11(4):717-728; Blanc V et al. (1996) Biochimie 78(6):511-517; Corneille S et al. (2001) Plant J 27:171-178).
b) Cytochrome P-450 enzymes in combination with the sulfonylurea pro-herbicide R7402 (2-methylethyl-2-3-dihydro-N-[(4,6-dimethoxypyrimidine-2-yl)aminocarbonyl]-1,2-benzoisothiazol-7-sulfonamid-1,1-dioxide) (O'Keefe D P et al. (1994) Plant Physiol 105:473-482; Tissier A F et al. (1999) Plant Cell 11:1841-1852; Koprek T et al. (1999) Plant J 19(6):719-726; O'Keefe D P (1991) Biochemistry 30(2):447-55).
c) Indoleacetic acid hydrolases like e.g., the tms2 gene product from Agrobacterium tumefaciens in combination with naphthalacetamide (NAM) (Fedoroff N V & Smith D L (1993) Plant J 3:273-289; Upadhyaya N M et al. (2000) Plant Mol Biol Rep 18:227-223; Depicker A G et al. (1988) Plant Cell rep 104:1067-1071; Karlin-Neumannn G A et al. (1991) Plant Cell 3:573-582; Sundaresan V et al. (1995) Gene Develop 9:1797-1810; Cecchini E et al. (1998) Mutat Res 401(1-2):199-206; Zubko E et al. (2000) Nat Biotechnol 18:442-445).
d) Haloalkane dehalogenases (dhlA gene product) from Xanthobacter autotropicus GJ10 in combination with 1,2-dichloroethane (DCE) (Naested H et al. (1999) Plant J 18(5)571-576; Janssen D B et al. (1994) Annu Rev Microbiol 48: 163-191; Janssen D B (1989) J Bacteriol 171(12):6791-9).
e) Thymidine kinases (TK), e.g., from Type 1 Herpes Simplex virus. (TK HSV-1), in combination with acyclovir, ganciclovir or 1,2-deoxy-2-fluoro-b-D-arabinofuranosil-5-iodouracile (FIAU) (Czako M & Marton L (1994) Plant Physiol 104:1067-1071; Wigler M et al. (1977) Cell 11(1):223-232; McKnight S L et al. (1980) Nucl Acids Res 8(24):5949-5964; McKnight S L et al. (1980) Nucl Acids Res 8(24):5931-5948; Preston et al. (1981) J Virol 38(2):593-605; Wagner et al. (1981) Proc Natl Acad Sci USA 78(3):1441-1445; St. Clair et al. (1987) Antimicrob Agents Chemother 31(6):844-849).

Several other counter-selection systems are known in the art (see for example international application WO 04/013333; p. 13 to 20 for a summary; hereby incorporated by reference).

However, the selection systems directed to the production of marker-free cells or organisms described in the art so far requires two separate selection-marker:
1. first, a negative selection marker (e.g., conferring resistance against a herbicide or a antibiotic), which allows for selection of cells which have incorporated the transformation construct, and
2. second, a counter-selection marker (see above) which allows for selection of cells which have successfully undergone deletion/excision of the marker sequences.

WO 03/060133 is describing enzymes like the D-amino acid oxidase from Rhodotorula gracilis. The toxic effect of certain amino acids can—depending on the amino acid—be lowered or increased by metabolization by e.g., a D-amino acid oxidase.

There is so far no combined negative/counter-selection systems described in the art which are based on dual-function marker making subsequent use of both its properties as a negative selection marker and a counter-selection marker. There is furthermore an unsatisfied demand—especially in the plant biotechnology area—for fast transformation systems leading to marker-free plants. It is therefore an objective of the present invention to provide an efficient negative/counter-selection system which allows for fast generation of marker-free transgenic plant cells and plants and which is based on a single dual-function marker. This objective has been achieved by the present invention.

### BRIEF DESCRIPTION OF THE INVENTION

Accordingly, a first embodiment of the invention relates to a method for producing a transgenic plant comprising:
i) transforming a plant cell with a first expression cassette comprising a nucleic acid sequence encoding a D-amino acid oxidase operably linked with a promoter allowing expression in plant cells or plants, in combination with at least one second expression cassette suitable for conferring to said plant an agronomically valuable trait, and
ii) providing at least one first compound X, which is phytotoxic against plant cells not functionally expressing said D-amino acid oxidase, wherein said compound X can be metabolized by said D-amino acid oxidase into one or more compound(s) Y which are non-phytotoxic or less phytotoxic than compound X, and
iii) treating said transformed plant cells of step i) with said first compound X in a phytotoxic concentration and selecting plant cells comprising in their genome both said first and said second expression cassette, wherein said first expression cassette is conferring resistance to said transformed plant cells against said compound X by expression of said D-amino acid oxidase, and
iv) providing at least one second compound M, which is non-phytotoxic or moderately phytotoxic against plant cells not functionally expressing said D-amino acid oxidase, wherein said compound M can be metabolized by said D-amino acid oxidase into one or more compound(s) N which are phytotoxic or more phytotoxic than compound M, and
v) breaking the combination between said first expression cassette and said second expression cassette and treating resulting said plant cells with said second compound M in a concentration toxic to plant cells still comprising said first expression cassette, and selecting plant cells comprising said second expression cassette but lacking said first expression cassette.

The first and the second expression cassette may not be combined on one DNA construct but may be employed in combination in form of—for example—a co-transformation approach wherein the two separate molecules are transformed together into the plant cells. In a scenario like this the combination of the first and the second expression cassette can be broken e.g. by segregation (for example following reproduction of resulting plantlets). In this scenario the multiplicity of resulting segregated transgenic plantlets can be easily screened for lack of the first expression cassette by employment of compound M.

However, the first and the second expression cassette may be combined on one DNA construct. Here the combination can be broken for example by means of sequence specific sequence deletion or excision e.g., by employing a sequence-specific recombinase or by induced sequence specific homologous recombination.

Accordingly, a second embodiment of the invention relates to a method for producing a transgenic plant comprising:
i) transforming a plant cell with a first DNA construct comprising
   a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
   b) at least one second expression cassette suitable for conferring to said plant an agronomically valuable trait, wherein said second expression cassette is not localized between said sequences which allow for specific deletion of said first expression cassette, and
ii) providing at least one first compound X, which is phytotoxic against plant cells not functionally expressing said D-amino acid oxidase, wherein said compound X can be metabolized by said D-amino acid oxidase into one or more compound(s) Y which are non-phytotoxic or less phytotoxic than compound X, and
iii) treating said transformed plant cells of step i) with said first compound X in a phytotoxic concentration and selecting plant cells comprising in their genome said first DNA construct, conferring resistance to said transformed plant cells against said compound X by expression of said D-amino acid oxidase, and
iv) providing at least one second compound M, which is non-phytotoxic or moderately phytotoxic against plant cells not functionally expressing said D-amino acid oxidase, wherein said compound M can be metabolized by said D-amino acid oxidase into one or more compound(s) N which are phytotoxic or more phytotoxic than compound M, and
v) inducing deletion of said first expression cassette from the genome of said trans-formed plant cells and treating said plant cells with said second compound M in a concentration toxic to plant cells still comprising said first expression cassette, thereby selecting plant cells comprising said second expression cassette but lacking said first expression cassette.

In a preferred embodiment the method of the invention further comprises the step of regeneration of a fertile plant. The method may further include sexually or asexually propagating or growing off-spring or a descendant of the plant regenerated from said plant cell.

In another preferred embodiment the first (phytotoxic) compound X is preferably comprising a D-amino acid structure selected from the group consisting of D-tryptophane, D-histidine, D-arginine, D-threonine, D-methionine, D-serine, and D-alanine; more preferably D-alanine, D-serine, and derivatives thereof. Most preferably, X is comprising and/or consisting of D-alanine, D-Serine, or derivatives thereof.

In another preferred embodiment the second (non-phytotoxic, but metabolizable into phytotoxic) compound M is preferably comprising a D-amino acid structure selected from the group consisting of D-isoleucine, D-valine, D-asparagine, D-leucine, D-lysine, D-proline, and D-glutamine; more preferably D-isoleucine, D-valine, and derivatives thereof. Most preferably, M is comprising and/or consisting of D-isoleucine, D-valine, or derivatives thereof.

Preferably, deletion of the first expression cassette can be realized by various means known in the art, including but not limited to one or more of the following methods:
a) recombination induced by a sequence specific recombinase, wherein said first expression cassette is flanked by corresponding recombination sites in a way that recombination between said flanking recombination sites results in deletion of the sequences in-between from the genome,
b) homologous recombination between homology sequences A and A′ flanking said first expression cassette, preferably induced by a sequence-specific double-strand break between said homology sequences caused by a sequence specific endonuclease, wherein said homology sequences A and A′ have sufficient length and homology in order to ensure homologous recombination between A and A′, and having an orientation which—upon recombination between A and A′—will lead to excision of said first expression cassette from the genome of said plant.

Various means are available for the person skilled in art to combine the deletion/excision inducing mechanism with the DNA construct of the invention comprising the D-amino acid oxidase dual-function selection marker. Preferably, a recombinase or endonuclease employable in the method of the invention can be expressed by a method selected from the group consisting of:
a) incorporation of a second expression cassette for expression of the recombinase or sequence-specific endonuclease operably linked to a plant promoter into said DNA construct, preferably together with said first expression cassette flanked by said sequences which allow for specific deletion,
b) incorporation of a second expression cassette for expression of the recombinase or sequence-specific endonuclease operably linked to a plant promoter into the plant cells or plants used as target material for the transformation thereby generating master cell lines or cells,
c) incorporation of a second expression cassette for expression of the recombinase or sequence-specific endonuclease operably linked to a plant promoter into a separate DNA construct, which is transformed by way of co-transformation with said first DNA construct into said plant cells,
d) incorporation of a second expression cassette for expression of the recombinase or sequence-specific endonuclease operably linked to a plant promoter into the plant cells or plants which are subsequently crossed with plants comprising the DNA construct of the invention.

In another preferred embodiment the mechanism of deletion/excision can be induced or activated in a way to prevent pre-mature deletion/excision of the dual-function marker. Preferably, thus expression and/or activity of an preferably employed sequence-specific recombinase or endonuclease can be induced and/or activated, preferably by a method selected from the group consisting of
a) inducible expression by operably linking the sequence encoding said recombinase or endonuclease to an inducible promoter,
b) inducible activation, by employing a modified recombinase or endonuclease comprising a ligand-binding-domain, wherein activity of said modified recombinase or endonuclease can by modified by treatment of a compound having binding activity to said ligand-binding-domain.

Preferably, thus the method of the inventions results in a plant cell or plant which is selection marker-free.

Another subject matter of the invention relates to DNA constructs which are suitable for employing in the method of the invention. A DNA construct suitable for use within the present invention is preferably comprising
a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette.

The D-amino acid oxidase expressed from the DNA-construct of the invention has preferably metabolising activity against at least one D-amino acid and comprises a sequences motive having the following consensus sequence:
[LIVM]-[LIVM]-H*-[NHA]-Y-G-x-[GSA]-[GSA]-x-G-x₅-G-x-A
   wherein amino acid residues given in brackets represent alternative residues for the respective position, x represents any amino acid residue, and indices numbers indicate the respective number of consecutive amino acid residues.

In an preferred embodiment D-amino acid oxidase expressed from the DNA-construct of the invention has preferably enzymatic activity against at least one of the amino acids selected from the group consisting of D-alanine, D-serine, D-isoleucine, D-valine, and derivatives thereof. Preferably said D-amino acid oxidase is selected from the group of amino acid sequences comprising
a) the sequences described by SEQ ID NO: 2, 4, 6, 8, 10, 12, and 14, and
b) the sequences having a sequence homology of at least 40%, preferably 60%, more preferably 80%, most preferably 95% with a sequence as described by SEQ ID NO: 2, 4, 6, 8, 10, 12, and 14, and
c) the sequences hybridizing under low or high stringency conditions—preferably under high stringency conditions—with a sequence as described by SEQ ID NO: 2, 4, 6, 8, 10, 12, and 14.

For ensuring marker deletion/excision the expression cassette for the D-amino acid oxidase (the first expression construct) comprised in the DNA construct of the invention is flanked by recombination sites for a sequence specific recombinase in a way the recombination induced between said flanking recombination sites results in deletion of the said first expression cassette from the genome.

Preferably said sequences which allow for specific deletion of said first expression cassette are selected from the group of sequences consisting of
a) recombination sites for a sequences-specific recombinase arranged in a way that recombination between said flanking recombination sites results in deletion of the sequences in-between from the genome, and
b) homology sequences A and A′ having a sufficient length and homology in order to ensure homologous recombination between A and A′, and having an orientation which—upon recombination between A and A′—will result in deletion of the sequences in-between from the genome.

There are various recombination sites and corresponding sequence specific recombinases known in the art, which can be employed for the purpose of the invention.

In a preferred embodiment, deletion/excision of the dual-marker sequence is deleted by homologous recombination induced by a sequence-specific double-strand break. The basic principals are disclosed in WO 03/004659. For this purpose the first expression construct (encoding for the dual-function marker) is flanked by homology sequences A and A′, wherein said homology sequences have sufficient length and homology in order to ensure homologous recombination between A and A′, and having an orientation which—upon recombination between A and A′—will lead to an excision of first expression cassette from the genome. Furthermore, the sequence flanked by said homology sequences further comprises at least one recognition sequence of at least 10 base pairs for the site-directed induction of DNA double-strand breaks by a sequence specific DNA double-strand break inducing enzyme, preferably a sequence-specific DNA-endonuclease, more preferably a homing-endonuclease, most preferably an endonuclease selected from the group consisting of I-SceI, I-CpaI, I-CpaII, I-CreI and I-ChuI or chimeras thereof with ligand-binding domains.

The expression cassette for the endonuclease or recombinase (comprising a sequence-specific recombinase or endonuclease operably linked to a plant promote) may be included in the DNA construct of the invention. Preferably, said second expression cassette is together with said first expression cassette flanked by said sequences which allow for specific deletion.

In another preferred embodiment, the expression and/or activity of said sequence-specific recombinase or endonuclease can be induced and/or activated for avoiding premature deletion/excision of the dual-function marker during a period where its action as a negative selection marker is still required. Preferably induction/activation can be realized by a method selected from the group consisting of
a) inducible expression by operably linking the sequence encoding said recombinase or endonuclease to an inducible promoter,
b) inducible activation, by employing a modified recombinase or endonuclease comprising a ligand-binding-domain, wherein activity of said modified recombinase or endonuclease can by modified by treatment of a compound having binding activity to said ligand-binding-domain.

Further embodiments of the inventions are related to transgenic vectors comprising a DNA construct of the invention. Transgenic cells or non-human organisms comprising a DNA construct or vector of the invention. Preferably said cells or non-human organisms are plant cells or plants, preferably plants which are of agronomical use.

The present invention enables generation of marker-free transgenic cells and organisms, preferably plants, an accurately predictable manner with high efficiency.

### GENERAL DEFINITIONS

The teachings, methods, sequences etc. employed and described in the international patent applications WO 03/004659, WO 04/013333, WO 03/060133 are hereby incorporated by reference.

To facilitate understanding of the invention, a number of terms are defined below. It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims. It must be noted that as used herein and in the appended claims, the singular forms “a” and “the” include plural reference unless the context clearly dictates otherwise. Thus, for example, reference to “a vector” is a reference to one or more vectors and includes equivalents thereof known to those skilled in the art, and so forth.

The term “about” is used herein to mean approximately, roughly, around, or in the region of. When the term “about” is used in conjunction with a numerical range, it modifies that range by extending the boundaries above and below the numerical values-set forth. In general, the term “about” is used herein to modify a numerical value above and below the stated value by a variance of 20 percent up or down (higher or lower).

As used herein, the word “or” means any one member of a particular list and also includes any combination of members of that list.

“Agronomically valuable trait” include any phenotype in a plant organism that is useful or advantageous for food production or food products, including plant parts and plant products. Non-food agricultural products such as paper, etc. are also included. A partial list of agronomically valuable traits includes pest resistance, vigor, development time (time to harvest), enhanced nutrient content, novel growth patterns, flavors or colors, salt, heat, drought and cold tolerance, and the like. Preferably, agronomically valuable traits do not include selectable marker genes (e.g., genes encoding herbicide or anti-biotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g. luciferase, glucuronidase, chloramphenicol acetyl transferase (CAT, etc.). Such agronomically valuable important traits may include improvement of pest resistance (e.g., Melchers et al. (2000) Curr Opin Plant Biol 3(2):147-52), vigor, development time (time to harvest), enhanced nutrient content, novel growth patterns, flavors or colors, salt, heat, drought, and cold tolerance (e.g., Sakamoto et al. (2000) J Exp Bot 51(342):81-8; Saijo et al. (2000) Plant J 23(3): 319-327; Yeo et al. (2000) Mol Cells 10(3):263-8; Cushman et al. (2000) Curr Opin Plant Biol 3(2):117-24), and the like. Those of skill will recognize that there are numerous polynucleotides from which to choose to confer these and other agronomically valuable traits.

As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The abbreviations used herein are conventional one letter codes for the amino acids: A, alanine; B, asparagine or aspartic acid; C, cysteine; D aspartic acid; E, glutarnate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid (see L. Stryer, Biochemistry, 1988, W. H. Freeman and Company, New York. The letter “x” as used herein within an amino acid sequence can stand for any amino acid residue.

The term “nucleotide sequence of interest” refers to any nucleotide sequence, the manipulation of which may be deemed desirable for any reason (e.g., confer improved qualities), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product, (e.g., promoter sequence, polyadenylation sequence, termination sequence, enhancer sequence, etc.). A nucleic acid sequence of interest may preferably encode for an agronomically valuable trait.

The term “nucleic acid” refers to deoxyribonucleotides or ribonucleotides and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”.

The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid which is transcribed and translated in a sequence-specific manner to produce into a particular polypeptide or protein when placed under the control of appropriate regulatory sequences. The coding region is said to encode such a polypeptide or protein.

A “polynucleotide construct” refers to a nucleic acid at least partly created by recombinant methods. The term “DNA construct” is referring to a polynucleotide construct consisting of deoxyribonucleotides. The construct may be single- or—preferably—double stranded. The construct may be circular or linear.

The skilled worker is familiar with a variety of ways to obtain one of a DNA construct. Constructs can be prepared by means of customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The term “sense” is understood to mean a nucleic acid having a sequence which is homologous or identical to a target sequence, for example a sequence which binds to a protein transcription factor and which is involved in the expression of a given gene. According to a preferred embodiment, the nucleic acid comprises a gene of interest and elements allowing the expression of the said gene of interest.

The term “antisense” is understood to mean a nucleic acid having a sequence complementary to a target sequence, for example a messenger RNA (mRNA) sequence the blocking of whose expression is sought to be initiated by hybridization with the target sequence.

As used herein, the terms “complementary” or “complementarity” are used in reference to nucleotide sequences related by the base-pairing rules. For example, the sequence 5′-AGT-3′ is complementary to the sequence 5′-ACT-3′. Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. A “complement” of a nucleic acid sequence as used herein refers to a nucleotide sequence whose nucleic acids show total complementarity to the nucleic acids of the nucleic acid sequence.

The term “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

The term “chromosomal DNA” or “chromosomal DNA-sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), and in situ PCR.

The term “gene” refers to a coding region operably joined to appropriate regulatory sequences capable of regulating the expression of the polypeptide in some manner. A gene includes untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, posttranscriptional cleavage and polyadenylation.

The term “expression construct” or “expression construct” as used herein is intended to mean the combination of any nucleic acid sequence to be expressed in operable linkage with a promoter sequence and—optionally—additional elements (like e.g., terminator and/or polyadenylation sequences) which facilitate expression of said nucleic acid sequence.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from plants or plant pathogens like e.g., plant viruses.

Promoters may be tissue specific or cell specific. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining (as described for example in Example 7) or immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (e.g., peroxidase conjugated) secondary antibody which is specific for the primary antibody is allowed to bind to the sectioned tissue and specific binding detected (e.g., with avidin/biotin) by microscopy. Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

Where expression of a gene in all tissues of a transgenic plant or other organism is desired, one can use a “constitutive” promoter, which is generally active under most environmental conditions and states of development or cell differentiation (Benfey et al. (1989) EMBO J. 8:2195-2202). The promoter controlling expression of the trait gene and/or selection marker can be constitutive. Suitable constitutive promoters for use in plants include, for example, the cauliflower mosaic virus (CaMV) 35S transcription initiation region (Franck et al. (1980) Cell 21:285-294; Odell et al. (1985) Nature 313:810-812; Shewmaker et al. (1985) Virology 140:281-288; Gardner et al. 1986, Plant Mol. Biol. 6, 221-228), the 19S transcription initiation region (U.S. Pat. No. 5,352,605 and WO 84/02913), and region VI promoters, the 1′- or 2′-promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, or the mannopine synthase promoter (MAS). Other constitutive plant promoters include various ubiquitin or polyubiquitin promoters derived from, inter alia, Arabidopsis (Sun and Callis (1997) Plant J 11(5): 1017-1027), the mas, Mac or DoubleMac promoters (U.S. Pat. No. 5,106,739; Comai et al. (1990) Plant Mol Biol 15:373-381), the ubiquitin promoter (Holtorf S et al. (1995) Plant Mol Biol 29:637-649) and other transcription initiation regions from various plant genes known to those of skill in the art. Useful promoters for plants also include those obtained from Ti- or Ri-plasmids, from plant cells, plant viruses or other organisms whose promoters are found to be functional in plants. Bacterial promoters that function in plants, and thus are suitable for use in the methods of the invention include the octopine synthetase promoter, the nopaline synthase promoter, and the mannopine synthetase promoter. Suitable endogenous plant promoters include the ribulose-1,6-biphosphate (RUBP) carboxylase small subunit (ssu) promoter, the α-conglycinin promoter, the phaseolin promoter, the ADH promoter, and heat-shock promoters. Further preferred constitutive promoters are the nitrilase promoter from Arabidopsis thaliana (WO 03/008596) and the Pisum sativum ptxA promoter (e.g., as incorporated in the construct described by SEQ ID NO: 15; base pair 6479-7341, complementary orientation).

Of course, promoters can regulate expression all of the time in only one or some tissues. Alternatively, a promoter can regulate expression in all tissues but only at a specific developmental time point. As noted above, the excision promoter (i.e., the promoter that is linked to the sequence-specific DNA cleaving polynucleotide) is generally not constitutive, but instead is active for only part of the life cycle or at least one tissue of the transgenic organism. One can use a promoter that directs expression of a gene of interest in a specific tissue or is otherwise under more precise environmental or developmental control. Examples of environmental conditions that may affect transcription by inducible promoters include pathogen attack, anaerobic conditions, ethylene or the presence of light. Promoters under developmental control include promoters that initiate transcription only in certain tissues or organs, such as leaves, roots, fruit, seeds, or flowers, or parts thereof. The operation of a promoter may also vary depending on its location in the genome. Thus, an inducible promoter may become fully or partially constitutive in certain locations.

Examples of tissue-specific plant promoters under developmental control include promoters that initiate transcription only in certain tissues, such as fruit, seeds, flowers, anthers, ovaries, pollen, the meristem, flowers, leaves, stems, roots and seeds. The tissue-specific ES promoter from tomato is particularly useful for directing gene expression so that a desired gene product is located in fruits. See, e.g., Lincoln et al. (1988) Proc Natl Acad Sci USA 84:2793-2797; Deikman et al. (1988) EMBO J. 7:3315-3320 Deikman et al. (1992) Plant Physiol 100:2013-2017. Other suitable seed specific promoters include those derived from the following genes: MAC1 from maize (Sheridan et al. (1996) Genetics 142:1009-1020, Cat3 from maize (GenBank No. L05934, Ableretal. (1993) Plant Mol Biol 22:10131-1038, the gene encoding oleosin 18 kD from maize (GenBank No. J05212, Lee et al. (1994) Plant Mol Biol 26:1981-1987), viviparous-1 from Arabidopsis (Genbank No. U93215), the gene encoding oleosin from Arabidopsis (Genbank No. Z17657), Atmycl from Arabidopsis (Urao et al. (1996) Plant Mol Biol 32:571-576, the 2s seed storage protein gene family from Arabidopsis (Conceicao et al. (1994) Plant 5:493-505) the gene encoding oleosin 20 kD from Brassica napus (GenBank No. M63985), napin from Brassica napus (GenBank No. J02798, Josefsson et al. (1987) J. Biol. Chem. 262:12196-12201), the napin gene family (e.g., from Brassica napus; Sjodahl et al. (1995) Planta 197:264-271, U.S. Pat. No. 5,608,152; Stalberg K, et al. (1996) L. Planta 199: 515-519), the gene encoding the 2S storage protein from Brassica napus (Dasgupta et al. (1993) Gene 133: 301-302), the genes encoding oleosin A (Genbank No. U09118) and oleosin B (Genbank No. U09119) from soybean, the gene encoding low molecular weight sulphur rich protein from soybean (Choi et al. (1995) Mol Gen Genet. 246:266-268), the phaseolin gene (U.S. Pat. No. 5,504,200, Bustos M M et al., Plant Cell. 1989; 1(9):839-53), the 2S albumin gene (Joseffson L G et al. (1987) J Biol Chem 262: 12196-12201), the legumin gene (Shirsat A et al. (1989) Mol Gen Genet. 215(2):326-331), the USP (unknown seed protein) gene (Baümlein H et al. (1991) Mol Gen Genetics 225(3):459-67), the sucrose binding protein gene (WO 00/26388), the legumin B4 gene (LeB4; Bäumlein H et al. (1991) Mol Gen Genet 225:121-128; Baeumlein et al. (1992) Plant J 2(2):233-239; Fiedler U et al. (1995) Biotechnology (NY) 13(10):1090-1093), the Ins Arabidopsis oleosin gene (WO9845461), the Brassica Bce4 gene (WO 91/13980), genes encoding the “high-molecular-weight glutenin” (HMWG), gliadin, branching enzyme, ADP-glucose pyrophosphatase (AGPase) or starch synthase. Furthermore preferred promoters are those which enable seed-specific expression in monocots such as maize, barley, wheat, rye, rice and the like. Promoters which may advantageously be employed are the promoter of the Ipt2 or Ipt1 gene (WO 95/15389, WO 95/23230) or the promoters described in WO 99/16890 (promoters of the hordein gene, the glutelin gene, the oryzin gene, the prolamine gene, the gliadin gene, the zein gene, the kasirin gene or the secalin gene).

Further suitable promoters are, for example, specific promoters for tubers, storage roots or roots such as, for example, the class I patatin promoter (B33), the potato cathepsin D inhibitor promoter, the starch synthase (GBSS1) promoter or the sporamin promoter, and fruit-specific promoters such as, for example, the tomato fruit-specific promoter (EP-A 409 625).

Promoters which are furthermore suitable are those which ensure leaf-specific expression. Promoters which may be mentioned are the potato cytosolic FBPase promoter (WO 98/18940), the Rubisco (ribulose-1,5-bisphosphate carboxylase) SSU (small subunit) promoter or the potato ST-LSI promoter (Stockhaus et al. (1989) EMBO J. 8(9):2445-2451). Other preferred promoters are those which govern expression in seeds and plant embryos.

Further suitable promoters are, for example, fruit-maturation-specific promoters such as, for example, the tomato fruit-maturation-specific promoter (WO 94/21794), flower-specific promoters such as, for example, the phytoene synthase promoter (WO 92/16635) or the promoter of the P-rr gene (WO 98/22593) or another node-specific promoter as described in EP-A 249676 may be used advantageously. The promoter may also be a pith-specific promoter, such as the promoter isolated from a plant TrpA gene as described in WO 93/07278. A development-regulated promoter is, inter alia, described by Baerson et al. (Baerson S R, Lamppa G K (1993) Plant Mol Biol 22(2):255-67).

Other preferred promoters are promoters induced by biotic or abiotic stress, such as, for example, the pathogen-inducible promoter of the PRP1 gene (Ward et al., Plant Mol Biol 1993, 22: 361-366), the tomato heat-inducible hsp80 promoter (U.S. Pat. No. 5,187,267), the potato chill-inducible alpha-amylase promoter (WO 96/12814) or the wound-induced pinII promoter (EP375091).

Promoters may also encompass further promoters, promoter elements or minimal promoters capable of modifying the expression-specific characteristics. Thus, for example, the tissue-specific expression may take place in addition as a function of certain stress factors, owing to genetic control sequences. Such elements are, for example, described for water stress, abscisic acid (Lam E and Chua N H (1991) J Biol Chem 266(26):17131-17135) and heat stress (Schoffl F et al. (1989) Molecular & General Genetics 217(2-3):246-53).

The term “operable linkage” or “operably linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. Operable linkage, and an expression construct, can be generated by means of customary recombination and cloning techniques as described (e.g., in Maniatis 1989; Silhavy 1984; Ausubel 1987; Gelvin 1990). However, further sequences which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of promoter and nucleic acid sequence to be expressed, can exist in a vector-integrated form and be inserted into a plant genome, for example by transformation.

The terms “polypeptide”, “peptide”, “oligopeptide”, “polypeptide”, “gene product”, “expression product” and “protein” are used interchangeably herein to refer to a polymer or oligomer of consecutive amino acid residues.

Preferably, the term “isolated” when used in relation to a nucleic acid, as in “an isolated nucleic acid sequence” refers to a nucleic acid sequence that is identified and separated from at least one contaminant nucleic acid with which it is ordinarily associated in its natural source. Isolated nucleic acid is nucleic acid present in a form or setting that is different from that in which it is found in nature. In contrast, non-isolated nucleic acids are nucleic acids such as DNA and RNA which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, an isolated nucleic acid sequence comprising SEQ ID NO:1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “wild-type”, “natural” or of “natural origin” means with respect to an organism, polypeptide, or nucleic acid sequence, that said organism is naturally occurring or available in at least one naturally occurring organism which is not changed, mutated, or otherwise manipulated by man.

“Transgene”, “transgenic” or “recombinant” refers to an polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples above.

The term “transgenic” or “recombinant” when used in reference to a cell refers to a cell which contains a transgene, or whose genome has been altered by the introduction of a transgene. The term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

The term “transgene” as used herein refers to any nucleic acid sequence which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence which contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc. Preferably, the term “transgenic” or “recombinant” with respect to a regulatory sequence (e.g., a promoter of the invention) means that said regulatory sequence is covalently joined and adjacent to a nucleic acid to which it is not adjacent in its natural environment.

The term “foreign gene” refers to any nucleic acid (e.g., gene sequence) which is introduced into the genome of a cell by experimental manipulations and may include gene sequences found in that cell so long as the introduced gene contains some modification (e.g., a point mutation, the presence of a selectable marker gene, etc.) relative to the naturally-occurring gene.

Preferably, the term “transgene” or “transgenic” with respect to, for example, a nucleic acid sequence (or an organism, expression construct or vector comprising said nucleic acid sequence) refers to all those constructs originating by experimental manipulations in which either
a) said nucleic acid sequence, or
b) a genetic control sequence linked operably to said nucleic acid sequence a), for example a promoter, or
c) (a) and (b)
   is not located in its natural genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1000 bp, very especially preferably at least 5000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815).

“Recombinant” polypeptides or proteins refer to polypeptides or proteins produced by recombinant DNA techniques, i.e., produced from cells transformed by an exogenous recombinant DNA construct encoding the desired polypeptide or protein. Recombinant nucleic acids and polypeptide may also comprise molecules which as such does not exist in nature but are modified, changed, mutated or otherwise manipulated by man.

The term “genetically-modified organism” or “GMO” refers to any organism that comprises transgene DNA. Exemplary organisms include plants, animals and microorganisms.

The terms “heterologous nucleic acid sequence” or “heterologous DNA” are used interchangeably to refer to a nucleotide sequence which is ligated to a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed.

The term “cell” or “plant cell” as used herein refers to a single cell. The term “cells” refers to a population of cells. The population may be a pure population comprising one cell type. Likewise, the population may comprise more than one cell type. In the pre-sent invention, there is no limit on the number of cell types that a cell population may comprise. The cells may be synchronize or not synchronized. A plant cell within the meaning of this invention may be isolated (e.g., in suspension culture) or comprised in a plant tissue, plant organ or plant at any developmental stage.

The term “organ” with respect to a plant (or “plant organ”) means parts of a plant and may include (but shall not limited to) for example roots, fruits, shoots, stem, leaves, anthers, sepals, petals, pollen, seeds, etc.

The term “tissue” with respect to a plant (or “plant tissue”) means arrangement of multiple plant cells including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissue may be in planta, in organ culture, tissue culture, or cell culture.

The term “plant” as used herein refers to a plurality of plant cells which are largely differentiated into a structure that is present at any stage of a plant's development. Such structures include one or more plant organs including, but are not limited to, fruit, shoot, stem, leaf, flower petal, etc.

The term “expression” refers to the biosynthesis of a gene product. For example, in the case of a structural gene, expression involves transcription of the structural gene into mRNA and—optionally—the subsequent translation of mRNA into one or more polypeptides.

The term “transformation” as used herein refers to the introduction of genetic material (e.g., a transgene) into a cell. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA) which detects the presence of a polypeptide encoded by one or more of the transgenes. Alternatively, transient transformation may be detected by detecting the activity of the protein (e.g., β-glucuronidase) encoded by the transgene (e.g., the uid A gene) as demonstrated herein [e.g., histochemical assay of GUS enzyme activity by staining with X-gluc which gives a blue precipitate in the presence of the GUS enzyme; and a chemiluminescent assay of GUS enzyme activity using the GUS-Light kit (Tropix)]. The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction of genomic DNA of the cell to amplify transgene sequences. The term “stable transformant” refers to a cell which has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression which may exhibit variable properties with respect to meiotic stability.

The terms “infecting” and “infection” with a bacterium refer to co-incubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium which causes crown gall. The term “Agtobacterium” includes, but is not limited to, the strains Agrobacterium tumefaciens, (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are herein incorporated by reference), and are commercially available (e.g., the helium gas-driven microprojectile accelerator (PDS-1000/He) (BioRad).

The term “microwounding” when made in reference to plant tissue refers to the introduction of microscopic wounds in that tissue. Microwounding may be achieved by, for example, particle bombardment as described herein.

The terms “homology” or “identity” when used in relation to nucleic acids refers to a degree of complementarity. Homology or identity between two nucleic acids is understood as meaning the identity of the nucleic acid sequence over in each case the entire length of the sequence, which is calculated by comparison with the aid of the program algorithm GAP (Wisconsin Package Version 10.0, University of Wisconsin, Genetics Computer Group (GCG), Madison, USA) with the parameters being set as follows:

| | |
|---|---|
| Gap Weight: 12 | Length Weight: 4 |
| Average Match: 2,912 | Average Mismatch: −2,003 |

For example, a sequence with at least 95% homology (or identity) to the sequence SEQ ID NO. 1 at the nucleic acid level is understood as meaning the sequence which, upon comparison with the sequence SEQ ID NO. 1 by the above program algorithm with the above parameter set, has at least 95% homology. There may be partial homology (i.e., partial identity of less then 100%) or complete homology (i.e., complete identity of 100%).

Alternatively, a partially complementary sequence is understood to be one that at least partially inhibits a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described infra. When used in reference to a single-stranded nucleic acid sequence, the term “substantially homologous” refers to any probe which can hybridize to the single-stranded nucleic acid sequence under conditions of low stringency as described infra.

The term “hybridization” as used herein includes “any process by which a strand of nucleic acid joins with a complementary strand through base pairing.” (Coombs 1994). Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl [see e.g., Anderson and Young, Quantitative Filter Hybridization, in Nucleic Acid Hybridization (1985)]. Other references include more sophisticated computations which take structural as well as sequence characteristics into account for the calculation of Tm.

Low stringency conditions when used in reference to nucleic acid hybridization comprise conditions equivalent to binding or hybridization at 68° C. in a solution consisting of 5×SSPE (43.8 g/L NaCl, 6.9 g/L NaH₂PO₄.H₂O and 1.85 g/L EDTA, pH adjusted to 7.4 with NaOH), 1% SDS, 5×Denhardt's reagent [50×Denhardt's contains the following per 500 mL: 5 g Ficoll (Type 400, Pharmacia), 5 g BSA (Fraction V; Sigma)] and 100 μg/mL denatured salmon sperm DNA followed by washing in a solution comprising 0.2×SSPE, and 0.1% SDS at room temperature when a DNA probe of about 100 to about 1000 nucleotides in length is employed.

High stringency conditions when used in reference to nucleic acid hybridization comprise conditions equivalent to binding or hybridization at 68° C. in a solution consisting of 5×SSPE, 1% SDS, 5×Denhardt's reagent and 100 μg/mL denatured salmon sperm DNA followed by washing in a solution comprising 0.1×SSPE, and 0.1% SDS at 68° C. when a probe of about 100 to about 1000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

### DETAILED DESCRIPTION OF THE INVENTION

Accordingly, a first embodiment of the invention relates to a method for producing a transgenic plant comprising:
i) transforming a plant cell with a first expression cassette comprising a nucleic acid sequence encoding a D-amino acid oxidase operably linked with a promoter allowing expression in plant cells or plants, in combination with at least one second expression cassette suitable for conferring to said plant an agronomically valuable trait, and
ii) providing at least one first compound X, which is phytotoxic against plant cells not functionally expressing said D-amino acid oxidase, wherein said compound X can be metabolized by said D-amino acid oxidase into one or more compound(s) Y which are non-phytotoxic or less phytotoxic than compound X, and
iii) treating said transformed plant cells of step i) with said first compound X in a phytotoxic concentration and selecting plant cells comprising in their genome both said first and said second expression cassette, wherein said first expression cassette is conferring resistance to said transformed plant cells against said compound X by expression of said D-amino acid oxidase, and
iv) providing at least one second compound M, which is non-phytotoxic or moderately phytotoxic against plant cells not functionally expressing said D-amino acid oxidase, wherein said compound M can be metabolized by said D-amino acid oxidase into one or more compound(s) N which are phytotoxic or more phytotoxic than compound M, and
v) breaking the combination between said first expression cassette and said second expression cassette and treating resulting said plant cells with said second compound M in a concentration toxic to plant cells still comprising said first expression cassette, and selecting plant cells comprising said second expression cassette but lacking said first expression cassette.

The term “combination” or “combined” with respect to the relation between the first and the second expression cassette is to be understood in the broad sense and is intended to mean any mode operation which is linking the functionality of the two expression cassettes. The first and the second expression cassette may be comprised in one DNA construct but may also be separate molecules.

Correspondingly the term “breaking the combination” is to be understood in the broad sense to comprise any method which leads to separation of the two expression cassettes. The person skilled in the art is aware of various means to separate sequences comprised in a genome, including but not limited to segregation, sequence excision or deletion etc.

The plant cell to be transformed can be an isolated cell but also part of a plant tissue, culture, organ or an entire plant at any developmental stage. Furthermore, the plant cell placed under negative selection or counter selection can be isolated cells but also part of a plant tissue, culture, organ or an entire plant at any developmental stage. Between the described steps of the method of the invention additional steps may be comprised. For example, between negative selection and counter selection there might be additional steps of e.g., induction of excision and/or plant regeneration.

As mentioned above, the first and the second expression cassette may not be combined on one DNA construct but may be employed in combination in form of—for example—a co-transformation approach wherein the two separate molecules are trans-formed together into the plant cells. In a scenario like this the combination of the first and the second expression cassette can be broken e.g. by segregation (for example following reproduction of resulting plantlets). In this scenario the multiplicity of resulting segregated transgenic plantlets can be easily screened for lack of the first expression cassette by employment of compound M, which can be applied, e.g., by spraying.

However, the first and the second expression cassette may be combined on one DNA construct. Here the combination can be broken for example by means of sequence specific sequence deletion or excision e.g., by employing a sequence-specific recombinase or by induced sequence specific homologous recombination.

Accordingly, a second embodiment of the invention relates to a method for producing a transgenic plant comprising:
i) transforming a plant cell with a first DNA construct comprising
   a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
   b) at least one second expression cassette suitable for conferring to said plant an agronomically valuable trait, wherein said second expression cassette is not localized between said sequences which allow for specific deletion of said first expression cassette, and
ii) providing at least one first compound X, which is phytotoxic against plant cells not functionally expressing said D-amino acid oxidase, wherein said compound X can be metabolized by said D-amino acid oxidase into one or more compound(s) Y which are non-phytotoxic or less phytotoxic than compound X, and
iii) treating said transformed plant cells of step i) with said first compound X in a phytotoxic concentration and selecting plant cells comprising in their genome said first DNA construct, conferring resistance to said transformed plant cells against said compound X by expression of said D-amino acid oxidase, and
iv) providing at least one second compound M, which is non-phytotoxic or moderately phytotoxic against plant cells not functionally expressing said D-amino acid oxidase, wherein said compound M can be metabolized by said D-amino acid oxidase into one or more compound(s) N which are phytotoxic or more phytotoxic than compound M, and
v) inducing deletion of said first expression cassette from the genome of said trans-formed plant cells and treating said plant cells with said second compound M in a concentration toxic to plant cells still comprising said first expression cassette, thereby selecting plant cells comprising said second expression cassette but lacking said first expression cassette.

In a preferred embodiment the method of the invention further comprises the step of regeneration of a fertile plant. The method may further include sexually or asexually propagating or growing off-spring or a descendant of the plant regenerated from said plant cell.

This invention discloses the subsequent use of a the marker gene dao1 encoding a D-amino acid oxidase (DAAO, EC 1.4.3.3) for both negative selection and counter-selection, depending on the substrate. DAAO catalyzes the oxidative deamination of a range of D-amino acids (Alonso J et al. (1998) Microbiol. 144, 1095-1101). Thus, the D-amino acid oxidase constitutes a dual-function marker. The marker has been successfully established in Arabidopsis thaliana, and proven to be versatile, rapidly yielding unambiguous results, and allowing selection immediately after germination (WO 03/060133)

Many prokaryotes and eukaryotes metabolize D-amino acids (Pilone M S (2000) Cell. Mol. Life. Sci. 57, 1732-174), but current information suggests that D-amino acid metabolism is severely restricted in plants.

However, studies of amino acid transporters in plants have shown that several of these proteins may mediate the transport of both L- and D-enantiomers of amino acids, although the latter usually at lower rates (Frommer W B et al. (1995) Proc. Natl. Acad. Sci. USA 92, 12036-12040; Boorer K J et al. (1996) J. Biol. Chem. 271, 2213-22203). These findings imply that plants absorb D-amino acids but metabolize few if any D-amino acids. D-amino acid catabolism follows several routes, one of the most common being oxidative deamination (Pilone M S (2000) Cell. Mol. Life. Sci. 57, 1732-1742). The natural occurrence of D-amino acids in plants is generally low, with measurable levels of D-alanine, D-serine, D-glutamine and D-asparagine but no detectable levels of D-valine and D-isoleucine (Bruckner H & Westhauser T (2003) Amino acids 24, 43-55). Hence, the amount and nature of substrates that DAAO may engage under natural conditions would not cause negative effects on plants.

In a preferred embodiment the first (phytotoxic) compound X is preferably comprising a D-amino acid structure selected from the group consisting of D-tryptophane, D-histidine, D-arginine, D-threonine, D-methionine, D-serine, and D-alanine; more preferably D-alanine, D-serine, and derivatives thereof. Most preferably, X is comprising and/or consisting of D-alanine, D-Serine, or derivatives thereof.

Within this invention it is demonstrated that the toxicity of D-amino acids like e.g., D-serine and D-alanine could be alleviated by the insertion of a gene encoding an enzyme that metabolizes D-amino acids. Wild-type A. thaliana were transformed with the dao1 gene from the yeast Rhodotorula gracilis under the control of the constitutive promoter CaMV 35S promoter. Exposure of this transgenic plant to D-alanine or D-serine showed that it could detoxify both of these D-amino acids (FIG. 4a,b).

In another preferred embodiment the second (non-phytotoxic, but metabolizable into phytotoxic) compound M is preferably comprising a D-amino acid structure selected from the group consisting of D-isoleucine, D-valine, D-asparagine, D-leucine, D-lysine, D-proline, and D-glutamine; more preferably D-isoleucine, D-valine, and derivatives thereof. Most preferably, M is comprising and/or consisting of D-isoleucine, D-valine, or derivatives thereof.

In contrast to D-amino acids like D-serine and D-alanine, other D-amino acids like D-valine and D-isoleucine, which are not toxic to wild-type plants, have a strong negative influence on the growth of plants expressing DAAO (FIG. 4c,d). The findings that DAAO expression mitigated the toxicity of D-serine and D-alanine, but induced metabolic changes that made D-isoleucine and D-valine toxic, demonstrate that the enzyme could provide a substrate-dependent, dual-function, selectable marker in plants. Selection is based on differences in the toxicity of different D-amino acids and their metabolites to plants. Thus, D-alanine and D-serine are toxic to plants, but are metabolized by DAAO into nontoxic products, whereas D-isoleucine and D-valine have low toxicity, but are metabolized by DAAO into the toxic keto acids 3-methyl-2-oxopentanoate and 3-methyl-2-oxobutanoate, respectively. Hence, both positive and negative selection is possible with the same marker gene, which is therefore considered a dual-function marker.

Another subject matter of the invention relates to DNA constructs which are suitable for employing in the method of the invention. A DNA construct suitable for use in the method of the invention is preferably comprising
a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette.

### I. THE DUAL-FUNCTION MARKER OF THE INVENTION

The term D-amino acid oxidase (abbreviated DAAO, DAMOX, or DAO) is referring to the enzyme coverting a D-amino acid into a 2-oxo acid, by—preferably—employing Oxygen (O₂) as a substrate and producing hydrogen peroxide (H₂O₂) as a co-product (Dixon M & Kleppe K. Biochim. Biophys. Acta 96 (1965) 357-367; Dixon M & Kleppe K Biochim. Biophys. Acta 96 (1965) 368-382; Dixon M & Kleppe Biochim. Biophys. Acta 96 (1965) 383-389; Massey V et al. Biochim. Biophys. Acta 48 (1961) 1-9. Meister A & Wellner D Flavoprotein amino acid oxidase. In: Boyer, P. D., Lardy, H. and Myrbäck, K. (Eds.), The Enzymes, 2nd ed., vol. 7, Academic Press, New York, 1963, p. 609-648.)

DAAO can be described by the Nomenclature Committee of the International Union of Biochemistry and Molecular Biology (IUBMB) with the EC (Enzyme Commission) number EC 1.4.3.3. Generally an DAAO enzyme of the EC 1.4.3.3. class is an FAD flavoenzyme that catalyzes the oxidation of neutral and basic D-amino acids into their corresponding keto acids. DAAOs have been characterized and sequenced in fungi and vertebrates where they are known to be located in the peroxisomes. The term D-amino oxidase further comprises D-aspartate oxidases (EC 1.4.3.1) (DASOX) (Negri A et al. (1992) J Biol. Chem. 267:11865-11871), which are enzymes structurally related to DAAO catalyzing the same reaction but active only toward dicarboxylic D-amino acids. Within this invention DAAO of the EC 1.4.3.3. class are preferred.

In DAAO, a conserved histidine has been shown (Miyano M et al. (1991) J Biochem 109:171-177) to be important for the enzyme's catalytic activity. In a preferred embodiment of the invention a DAAO is referring to a protein comprising the following consensus motive:
[LIVM]-[LIVM]-H*-[NHA]-Y-G-x-[GSA]-[GSA]-x-G-x₅-G-x-A
   wherein amino acid residues given in brackets represent alternative residues for the respective position, x represents any amino acid residue, and indices numbers indicate the respective number of consecutive amino acid residues. The abbreviation for the individual amino acid residues have their standard IUPAC meaning as defined above. A Clustal multiple alignment of the characteristic active site from various D-amino acids is shown in FIG. 1. Further potential DAAO enzymes comprising said motif are described in table below:

TABLE 1
Suitable D-amino acid oxidases from various organism. Acc.-No. refers to protein
sequence from SwisProt database.

| Acc.-No. | Gene Name | Description | Source Organism | Length |
|---|---|---|---|---|
| Q19564 | F18E3.7 | Putative D-amino acid oxidase | Caenorhabditis elegans | 334 |
| | | (EC 1.4.3.3) (DAMOX) (DAO) | | |
| | | (DAAO) | | |
| P24552 | | D-amino acid oxidase (EC | Fusarium solani | 361 |
| | | 1.4.3.3) (DAMOX) (DAO) (DAAO) | (subsp. pisi) (Nectria | |
| | | | haematococca) | |
| P14920 | DAO, DAMOX | D-amino acid oxidase (EC | Homo sapiens (Human) | 347 |
| | | 1.4.3.3) (DAMOX) (DAO) (DAAO) | | |
| P18894 | DAO, DAO1 | D-amino acid oxidase (EC | Mus musculus (Mouse) | 346 |
| | | 1.4.3.3) (DAMOX) (DAO) (DAAO) | | |
| P00371 | DAO | D-amino acid oxidase (EC | Sus scrofa (Pig) | 347 |
| | | 1.4.3.3) (DAMOX) (DAO) (DAAO) | | |
| P22942 | DAO | D-amino acid oxidase (EC | Oryctolagus cuniculus | 347 |
| | | 1.4.3.3) (DAMOX) (DAO) (DAAO) | (Rabbit) | |
| O35078 | DAO | D-amino acid oxidase (EC | Rattus norvegicus | 346 |
| | | 1.4.3.3) (DAMOX) (DAO) (DAAO) | (Rat) | |
| P80324 | DAO1 | D-amino acid oxidase (EC | Rhodosporidium | 368 |
| | | 1.4.3.3) (DAMOX) (DAO) (DAAO) | toruloides (Yeast) | |
| | | | (Rhodotorula gracilis) | |
| U60066 | DAO | D-amino acid oxidase (EC | Rhodosporidium | 368 |
| | | 1.4.3.3) (DAMOX) (DAO) (DAAO) | toruloides, strain | |
| | | | TCC 26217 | |
| Q99042 | DAO1 | D-amino acid oxidase (EC | Trigonopsis variabilis | 356 |
| | | 1.4.3.3) (DAMOX) (DAO) (DAAO) | (Yeast) | |
| P31228 | DDO | D-aspartate oxidase (EC | Bos taurus (Bovine) | 341 |
| | | 1.4.3.1) (DASOX) (DDO) | | |
| Q99489 | DDO | D-aspartate oxidase (EC | Homo sapiens (Human) | 341 |
| | | 1.4.3.1) (DASOX) (DDO) | | |
| Q9C1L2 | NCU06558.1 | (AF309689) putative D-amino | Neurospora crassa | 362 |
| | | acid oxidase G6G8.6 (Hypothetical | | |
| | | protein) | | |
| Q7SFW4 | NCU03131.1 | Hypothetical protein | Neurospora crassa | 390 |
| Q8N552 | | Similar to D-aspartate oxidase | Homo sapiens (Human) | 369 |
| Q7Z312 | DKFZP686F04272 | Hypothetical protein | Homo sapiens (Human) | 330 |
| | | DKFZp686F04272 | | |
| Q9VM80 | CG11236 | CG11236 protein (GH12548p) | Drosophila melanogaster | 341 |
| | | | (Fruit fly) | |
| O01739 | F20H11.5 | F20H11.5 protein | Caenorhabditis elegans | 383 |
| O45307 | C47A10.5 | C47A10.5 protein | Caenorhabditis elegans | 343 |
| Q8SZN5 | CG12338 | RE73481p | Drosophila melanogaster | 335 |
| | | | (Fruit fly) | |
| Q9V5P1 | CG12338 | CG12338 protein (RE49860p) | Drosophila melanogaster | 335 |
| | | | (Fruit fly) | |
| Q86JV2 | | Similar to Bos taurus (Bovine). | Dictyostelium discoideum | 599 |
| | | D-aspartate oxidase (EC | (Slime mold) | |
| | | 1.4.3.1) (DASOX) (DDO) | | |
| Q95XG9 | Y69A2AR.5 | Hypothetical protein | Caenorhabditis elegans | 322 |
| Q7Q7G4 | AGCG53627 | AgCP5709 (Fragment) | Anopheles gambiae | 344 |
| | | | str. PEST | |
| Q7PWY8 | AGCG53442 | AgCP12432 (Fragment) | Anopheles gambiae | 355 |
| | | | str. PEST | |
| Q7PWX4 | AGCG45272 | AgCP12797 (Fragment) | Anopheles gambiae | 373 |
| | | | str. PEST | |
| Q8PG95 | XAC3721 | D-amino acid oxidase | Xanthomonas axonopodis | 404 |
| | | | (pv. citri) | |
| Q8P4M9 | XCC3678 | D-amino acid oxidase | Xanthomonas campestris | 405 |
| | | | (pv. campestris) | |
| Q9X7P6 | SCO6740, | Putative D-amino acid oxidase | Streptomyces coelicolor | 320 |
| | SC5F2A.23C | | | |
| Q82MI8 | DAO, | Putative D-amino acid oxidase | Streptomyces avermitilis | 317 |
| | SAV1672 | | | |
| Q8VCW7 | DAO1 | D-amino acid oxidase | Mus musculus | 345 |
| | | | (Mouse) | |
| Q9Z302 | | D-amino acid oxidase | Cricetulus griseus | 346 |
| | | | (Chinese hamster) | |
| Q9Z1M5 | | D-amino acid oxidase | Cavia porcellus | 347 |
| | | | (Guinea pig) | |
| Q922Z0 | | Similar to D-aspartate oxidase | Mus musculus | 341 |
| | | | (Mouse) | |
| Q8R2R2 | | Hypothetical protein | Mus musculus | 341 |
| | | | (Mouse) | |
| P31228 | | D-aspartate oxidase | B. taurus | 341 |

D-Amino acid oxidase (EC-number 1.4.3.3) can be isolated from various organisms, including but not limited to pig, human, rat, yeast, bacteria or fungi. Example organisms are Candida tropicalis, Trigonopsis variabilis, Neurospora crassa, Chlorella vulgaris, and Rhodotorula gracilis. A suitable D-amino acid metabolising polypeptide may be an eukaryotic enzyme, for example from a yeast (e.g. Rhodotorula gracilis), fungus, or animal or it may be a prokaryotic enzyme, for example, from a bacterium such as Escherichia coli. Examples of suitable polypeptides which metabolize D-amino acids are shown in Table 1 and Table 2.

TABLE 2
Suitable D-amino acid oxidases from various organism. Acc.-No.
refers to protein sequence from SwisProt database.

| | |
|---|---|
| Q19564 | Caenorhabditis elegans. F18E3.7. |
| P24552 | Fusarii solani (subsp. pisi) (Nectria haematococca). |
| JX0152 | Fusarium solani |
| P14920 | Homo sapiens (Human) |
| P18894 | Mus musculus (mouse) |
| P00371 | Sus scrofa (pig) |
| P22942 | Oryctolagus cuniculus (Rabbit) |
| O35078 | Rattus norvegicus (Rat) |
| P80324 | Rhodosporidium toruloides (Yeast) (Rhodotorula gracilis) |
| Q99042 | Trigonopsis variabilis |
| Q9Y7N4 | Schizosaccharomyces pombe (Fission yeast) SPCC1450 |
| O01739 | Caenorhabditis elegans.F20H11.5 |
| Q28382 | Sus scrofa (Pig). |
| O33145 | Mycobacterium leprae |
| Q9X7P6 | Streptomyces coelicolor.SCSF2A.23C |
| Q9JXF8 | Neisseria meningitidis (serogroup B). |
| Q9Z302 | Cricetulus griseus (Chinese hamster) |
| Q921M5 | D-AMINO ACID OXIDASE. Cavia parcellus (Guinea pig) |

Preferably the D-amino acid oxidase is selected from the enzymes encoded by a nucleic acid sequence or a corresponding amino acid sequences selected from the following table 3:

TABLE 3
Suitable D-amino acid oxidases from various organism. Acc.-No.
refers to protein sequence from GenBank database.

| GenBanc | | |
|---|---|---|
| Acc.-No | Organism | SEQ ID |
| U60066 | Rhodosporidium toruloides (Yeast) | SEQ ID NO: 1, 2 |
| Z71657 | Rhodotorula gracilis | |
| A56901 | Rhodotorula gracilis | |
| AF003339 | Rhodosporidium toruloides | |
| AF003340 | Rhodosporidium toruloides | |
| U53139 | Caenorhabditis elegans | SEQ ID NO: 3, 4 |
| D00809 | Nectria haematococca | SEQ ID NO: 5, 6 |
| Z50019. | Trigonopsis variabilis | SEQ ID NO: 7, 8 |
| NC_003421 | Schizosaccharomyces pombe | SEQ ID NO: 9, 10 |
| | (fission yeast) | |
| AL939129. | Streptomyces coelicolor A3(2) | SEQ ID NO: 11, 12 |
| AB042032 | Candida boidinii | SEQ ID NO: 13, 14 |

DAAO is a well-characterized enzyme, and both its crystal structure and its catalytic mechanism have been determined by high-resolution X-ray spectroscopy (Umhau S. et al. (2000) Proc. Natl. Acad. Sci. USA 97, 12463-12468). It is a flavoenzyme located in the peroxisome, and its recognized function in animals is detoxification of D-amino acids (Pilone M S (2000) Cell. Mol. Life. Sci. 57, 1732-174). In addition, it enables yeasts to use D-amino acids for growth (Yurimoto H et al. (2000) Yeast 16, 1217-1227). As demonstrated above, DAAO from several different species have been characterized and shown to differ slightly in substrate affinities (Gabler M et al. (2000) Enzyme Microb. Techno. 27, 605-611), but in general they display broad substrate specificity, oxidatively deaminating all D-amino acids (except D-glutamate and D-aspartate for EC 1.4.3.3. calss DAAO enzymes; Pilone M S (2000) Cell. Mol. Life. Sci. 57, 1732-174).

DAAO activity is found in many eukaryotes (Pilone M S (2000) Cell. Mol. Life. Sci. 57, 1732-174), but there is no report of DAAO activity in plants. The low capacity for D-amino acid metabolism in plants has major consequences for the way plants respond to D-amino acids. For instance, the results provided herein demonstrate that growth of A. thaliana in response to D-serine and/or D-alanine is inhibited even at quite low concentrations (FIG. 1a,b). On the other hand, some D-amino acids, like D-valine and D-isoleucine, have minor effects on plant growth (FIG. 1c,d) per se, but can be converted into toxic metabolites by action of a DAAO.

In an preferred embodiment D-amino acid oxidase expressed form the DNA-construct of the invention has preferably enzymatic activity against at least one of the amino acids selected from the group consisting of D-alanine, D-serine, D-isoleucine, D-valine, and derivatives thereof. Preferably said D-amino acid oxidase is selected from the group of amino acid sequences comprising
a) the sequences described by SEQ ID NO: 2, 4, 6, 8, 10, 12, and 14, and
b) the sequences having a sequence homology of at least 40%, preferably 60%, more preferably 80%, most preferably 95% with a sequence as described by SEQ ID NO: 2, 4, 6, 8, 10, 12, and 14, and
c) the sequences hybridizing under low or high stringency conditions—preferably under high stringency conditions—with a sequence as described by SEQ ID NO: 2, 4, 6, 8, 10, 12, and 14.

Suitable D-amino acid oxidases also include fragments, mutants, derivatives, variants and alleles of the polypeptides exemplified above. Suitable fragments, mutants, derivatives, variants and alleles are those which retain the functional characteristics of the D-amino acid oxidase as defined above. Changes to a sequence, to produce a mutant, variant or derivative, may be by one or more of addition, insertion, deletion or substitution of one or more nucleotides in the nucleic acid, leading to the addition, insertion, deletion or substitution of one or more amino acids in the encoded polypeptide. Of course, changes to the nucleic acid that make no difference to the encoded amino acid sequence are included.

The D-amino acid oxidase of the invention may be expressed in the cytosol, peroxisome, or other intracellular compartment of the plant cell. Compartmentalisation of the D-amino acid metabolising polypeptide may be achieved by fusing the nucleic acid sequence encoding the DAAO polypeptide to a sequence encoding a transit peptide to generate a fusion protein. Gene products expressed without such transit peptides generally accumulate in the cytosol. The localisation of expressed DAAO in the peroxisome produces H₂0₂ that can be metabolised by the H₂0₂ degrading enzyme catalase. Higher levels of D-amino acids may therefore be required to produce damaging levels of H₂0₂. Expression of DAAO in the cytosol, where levels of catalase activity are lower, reduces the amount of D-amino acid required to produce damaging levels H₂0₂. Expres7sion of DAAO in the cytosol may be achieved by removing peroxisome targeting signals or transit peptides from the encoding nucleic acid sequence. For example, the dao1 gene (EC: 1.4.3.3: GenBank Acc.-No.: U60066) from the yeast Rhodotorula gracilis (Rhodosporidium toruloides) was cloned as described (WO 03/060133). The last nine nucleotides encode the signal peptide SKL, which guides the protein to the peroxisome sub-cellular organelle. Although no significant differences were observed between cytosolic and peroxisomal expressed DAAO, the peroxisomal construction was found to be marginally more effective than the cytosolic version in respect of inhibiting the germination of the DAAO transgenic plants on 30 mM D-Asn. However, both constructs are inhibited significantly more than the wild-type and may thus be used for conditional counter-selection.

### I.1 The Compounds X and M

The term “Compound X” means one or more chemical substances (i.e. one chemical compound or a mixture of two or more compound) which is phytotoxic against plant cells not functionally expressing the D-amino acid oxidase expressed from the first expression cassette of the invention, and which can be metabolized by said D-amino acid oxidase into one or more compound(s) Y which are non-phytotoxic or less phytotoxic than compound X.

The term “phytotoxic”, “phytotoxicity” or “phytotoxic effect” as used herein is intended to mean any measurable, negative effect on the physiology of a plant or plant cell resulting in symptoms including (but not limited to) for example reduced or impaired growth, reduced or impaired photosynthesis, reduced or impaired cell division, reduced or impaired regeneration (e.g., of a mature plant from a cell culture, callus, or shoot etc.), reduced or impaired fertility etc. Phytotoxicity may further include effects like e.g., necrosis or apoptosis. In an preferred embodiment results in an reduction of growth or regenerability of at least 50%, preferably at least 80%, more preferably at least 90% in comparison with a plant which was not treated with said phytotoxic compound.

The phytotoxic compound X is metabolized by said D-amino acid oxidase into one or more compound(s) Y which are non-phytotoxic or less phytotoxic than compound X. In an improved embodiment the toxicity (as for example assessed by one of the physiological indicators exemplified above like e.g., growth or regenerability) of the phytotoxic compound is reduced by the conversion to at least 50%, preferably at least 80%, more preferably at least 90% of the original phytotoxicity imposed by compound X. More preferred this reduction results in an phytotoxic effect on plants (or plant cells) functionally expressing said D-amino acid oxidase and treated with said compound X in comparison with plants (or plant cells; regardless whether expressing said D-amino acid oxidase or not) not treated with said compound X of not more then 30%, preferably not more then 15%, more preferably not more then 10%, most preferably no statistically significant difference in physiology can be observed.

The term “Compound M” means one or more chemical substances (i.e. one chemical compound or a mixture of two or more compounds) which is non-phytotoxic or moderately phytotoxic against plant cells not functionally expressing said D-amino acid oxidase, and which can be metabolized by said D-amino acid oxidase into one or more compound(s) N which are phytotoxic or more phytotoxic than compound M.

The term “phytotoxic”, “phytotoxicity” or “phytotoxic effect” has the same definition as given above.

The term “non-phytotoxic” means that no statistically significant difference in physiology can be observed between plant cells or plants (not comprising a functional D-amino acid oxidase) and the same plant cells or plants treated with compound M or untreated plants.

The term “moderate phytotoxic” means an reduction of an physiological indicator (as exemplified above like e.g., growth or regenerability) for treated plant cells or plants—not comprising a functional D-amino acid oxidase—in comparison with untreated plants or plant cells (regardless whether expressing said D-amino acid oxidase or not) not irreversibly effecting growth and/or performance of said treated plants or plant cells (but using the compound in a concentration sufficient to allow for distinguishing and/or separating transgenic plants (i.e., comprising said dual function marker) from non-transgenic plants (i.e., not comprising said marker)). Preferably, the reduction of an physiological indicator for said treated plant cells is not more then 30%, preferably not more then 15%, more preferably not more then 10%.

The phytotoxic compound M is metabolized by said D-amino acid oxidase into one or more compound(s) N which are phytotoxic or more phytotoxic than compound M. In an improved embodiment the toxicity (as for example assessed by one of the physiological indicators exemplified above like e.g., growth or regenerability) of the compound M is increased in a way that one or more physiological indicator (as exemplified above like e.g., growth or regenerability) are reduced by at least 20%, preferably at least 40%, more preferably at least 60%, most preferably at least 90%. The phytotoxic effect of compound N in comparison to compound M is increased by at least 100% (i.e. twice), preferably at least 500% (i.e. 5-times), more preferably at least 1000% (i.e. 10 times).

Various chemical compounds and mixtures thereof can be used as compound X or M. The person skilled in the art is aware of assay systems to assess the phytotoxicity of these compounds and the capability of a D-amino oxidase to metabolize said compounds in a way described above leading to decreased or increased phytotoxicity.

Preferably at least one of the chemical substances comprised in compound X and/or M comprises a D-amino acid structure.

As used herein the term a “D-amino acid structure” (such as a “D-leucine structure”, a “D-phenylalanine structure” or a “D-valine structure”) is intended to include the D-amino acid, as well as analogues, derivatives and mimetics of the D-amino acid that maintain the functional activity of the compound (discussed further below). For example, the term “D-phenylalanine structure” is intended to include D-phenylalanine as well as D-pyridylalanine and D-homophenylalanine. The term “D-leucine structure” is intended to include D-leucine, as well as substitution with D-valine or other natural or non-natural amino acid having an aliphatic side chain, such as D-norleucine. The term “D-valine structure” is intended to include D-valine, as well as substitution with D-leucine or other natural or non-natural amino acid having an aliphatic side chain.

The D-amino acid employed may be modified by an amino-terminal or an carboxy-terminal modifying group. The amino-terminal modifying group may be—for example—selected from the group consisting of phenylacetyl, diphenylacetyl, triphenylacetyl, butanoyl, isobutanoyl hexanoyl, propionyl, 3-hydroxybutanoyl, 4-hydroxybutanoyl, 3-hydroxypropionoyl, 2,4-dihydroxybutyroyl, 1-Adamantanecarbonyl, 4-methylvaleryl, 2-hydroxyphenylacetyl, 3-hydroxyphenylacetyl, 4-hydroxyphenylacetyl, 3,5-dihydroxy-2-naphthoyl, 3,7-dihydroxy-2-napthoyl, 2-hydroxycinnamoyl, 3-hydroxycinnamoyl, 4-hydroxycinnamoyl, hydrocinnamoyl, 4-formylcinnamoyl, 3-hydroxy-4-methoxycinnamoyl, 4-hydroxy-3-methoxycinnamoyl, 2-carboxycinnamoyl, 3,4,-dihydroxyhydrocinnamoyl, 3,4-dihydroxycinnamoyl, trans-Cinnamoyl, (.+−.)-mandelyl. (.+−.)-mandelyl-(.+−.)-mandelyl, glycolyl, 3-formylbenzoyl, 4-formylbenzoyl, 2-formylphenoxyacetyl, 8-formyl-1-napthoyl, 4-(hydroxymethyl)benzoyl, 3-hydroxybenzoyl, 4-hydroxybenzoyl, 5-hydantoinacetyl, L-hydroorotyl, 2,4-dihydroxybenzoyl, 3-benzoylpropanoyl, (.+−.)-2,4-dihydroxy-3,3-dimethylbutanoyl, DL-3-(4-hydroxyphenyl)lactyl, 3-(2-hydroxyphenyl)propionyl, 4-(2-hydroxyphenyl)propionyl, D-3-phenyllactyl, 3-(4-hydroxyphenyl)propionyl, L-3-phenyllactyl, 3-pyridylacetyl, 4-pyridylacetyl, isonicotinoyl, 4-quinolinecarboxyl, 1-isoquinolinecarboxyl and 3-isoquinolinecarboxyl. The carboxy-terminal modifying group may be—for example—selected from the group consisting of an amide group, an alkyl amide group, an aryl amide group and a hydroxy group.

The terms “analogue”, “derivative” and “mimetic” as used herein are intended to include molecules which mimic the chemical structure of a D-amino acid structure and retain the functional properties of the D-amino acid structure. Approaches to designing amino acid or peptide analogs, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

As used herein, a “derivative” of a compound X or M (e.g., a D-amino acid) refers to a form of X or M in which one or more reaction groups on the compound have been derivatized with a substituent group. Examples of peptide derivatives include peptides in which an amino acid side chain, or the amino- or carboxy-terminus has been derivatized. As used herein an “analogue” of a compound X or M refers to a compound which retains chemical structures of X or M necessary for functional activity of X or M yet which also contains certain chemical structures which differ from X or M, respectively. As used herein, a “mimetic” of a compound X or M refers to a compound in which chemical structures of X or M necessary for functional activity of X or M have been replaced with other chemical structures which mimic the conformation of X or M, respectively.

Analogues are intended to include compounds in which one or more D-amino acids are substituted with a homologous amino acid such that the properties of the original compound are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Non-limiting examples of homologous substitutions that can be made include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain.

Other possible modifications include N-alkyl (or aryl) substitutions, or backbone crosslinking to construct lactams and other cyclic structures. Other derivatives include C-terminal hydroxymethyl derivatives, O-modified derivatives (e.g., C-terminal hydroxymethyl benzyl ether), N-terminally modified derivatives including substituted amides such as alkylamides and hydrazides.

In certain embodiments the D-amino acid structure is coupled directly or indirectly to at least one modifying group (abbreviated as MG). The term “modifying group” is intended to include structures that are directly attached to the D-amino acid structure (e.g., by covalent coupling), as well as those that are indirectly attached (e.g., by a stable non-covalent association or by covalent coupling to additional amino acid residues). For example, the modifying group can be coupled to the amino-terminus or carboxy-terminus of a D-amino acid structure. Modifying groups covalently coupled to the D-amino acid structure can be attached by means and using methods well known in the art for linking chemical structures, including, for example, amide, alkylamino, carbamate, urea or ester bonds. In a preferred embodiment, the modifying group(s) comprises a cyclic, heterocyclic, polycyclic or branched alkyl group.

No endogenous D-amino acid oxidase activity has been reported in plants. Compound X or M, respectively, as substrates for the D-amino acid oxidase may be a D-amino acid structure comprising the structure of D-arginine, D-glutamate, D-alanine, D-aspartate, D-cysteine, D-glutamine, D-histidine, D-isoleucine, D-leucine, D-lysine, D-methionine, D-asparagine, D-phenylalanine, D-proline, D-serine, D-threonine, D-tryptophane, D-tyrpsine or D-valine. Preferably compound X and M is comprising D-arginine, D-glutamate, D-alanine, D-aspartate, D-cysteine, D-glutamine, D-histidine, D-isoleucine, D-leucine, D-lysine, D-methionine, D-asparagine, D-phenylalanine, D-proline, D-serine, D-threonine, D-tryptophane, D-tyrosine or D-valine. Other suitable substrates for D-amino acid metabolising enzymes include non-protein dextrorotatory amino acids, precursors of dextrorotatory amino acids and dextrorotatory amino acid derivatives. Suitable precursors include D-ornithine and D-citrulline.

The fact that compound X and M preferably comprise a D-amino acid structure does not rule out the presence of L-amino acid structures or L-amino acids. For some applications it may be preferred (e.g., for cost reasons) to apply a racemic mixture of D- and L-amino acids (or a mixture with enriched content of D-amino acids). Preferably, the ratio of the D-amino acid to the corresponding L-enantiomer is at least 1:1, preferably 2:1, more preferably 5:1, most preferably 10:1 or 100:1.

The preferred compound may be used in isolated form or in combination with other substances. For the purpose of application, the compound X or M are advantageously used together with the adjuvants conventionally employed in the art of formulation, and are therefore formulated in known manner, e.g. into emulsifiable concentrates, coatable pastes, directly sprayable or dilutable solutions, dilute emulsions, wettable powders, soluble powders, dusts, granulates, and also encapsulations in e.g. polymer substances. As with the nature of the compositions to be used, the methods of application, such as spraying, atomizing, dusting, scattering, coating or pouring, are chosen in accordance with the intended objectives and the prevailing circumstances.

The formulations, i.e. the compositions, preparations or mixtures containing compound X or M (active ingredient), and, where appropriate, a solid or liquid adjuvant, are pre-pared in known manner, e.g. by homogeneously mixing and/or grinding the active ingredients with extenders, e.g. solvents, solid carriers and, where appropriate, surface-active compounds (surfactants).

Suitable solvents are: aromatic hydrocarbons, preferably the fractions containing 8 to 12 carbon atoms, e.g. xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, ethylene glycol monomethyl or monoethyl ether, ketones such as cyclohexanone, strongly polar solvents such as N-methyl-2-pyrrolidone, dimethyl sulfoxide or dimethylformamide, as well as vegetable oils or epoxidized vegetable oils, such as epoxidized coconut oil or soybean oil; or—preferably—water.

The solid carriers used e.g. for dusts and dispersible powders are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable non-sorbent carriers are, for example, calcite or sand. In addition, a great number of pre-granulated materials of inorganic or organic nature can be used, e.g. especially dolomite or pulverized plant residues.

Depending on the nature of the compound X or M to be formulated suitable surface-active compounds are nonionic, cationic and/or anionic surfactants having good emulsifying, dispersing and wetting properties. The term “surfactants” will also be understood as comprising mixtures of surfactants.

Both so-called water-soluble soaps and also water-soluble synthetic surface-active compounds are suitable anionic surfactants. Suitable soaps are the alkali metal salts, alkaline earth metal salts or unsubstituted or substituted ammonium salts of higher fatty acids (C₁₀-C₂₂), e.g. the sodium or potassium salts of oleic or stearic acid or of natural fatty acid mixtures which can be obtained e.g. from coconut oil or tallow oil. Fatty acid methyltaurin salts may also be mentioned as surfactants.

More frequently, however, so-called synthetic surfactants are used, especially fatty sulfonates, fatty sulfates, sulfonated benzimidazole derivatives or alkylarylsulfonates.

The fatty sulfonates or sulfates are usually in the form of alkali metal salts, alkaline earth metal salts or unsubstituted or substituted ammonium salts and contain a C.sub.8-C.sub.22 alkyl radical which also includes the alkyl moiety of acyl radicals, e.g. the sodium or calcium salt of lignosulfonic acid, of dodecylsulfate or of a mixture of fatty alcohol sulfates obtained from natural fatty acids. These compounds also comprise the salts of sulfated and sulfonated fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a condensate of naphthalenesulfonic acid and formaldehyde. Also suitable are corresponding phosphates, e.g. salts of the phosphoric acid ester of an adduct of p-nonylphenol with 4 to 14 moles of ethylene oxide, or phospholipids.

Non-ionic surfactants are preferably polyglycol ether derivatives of aliphatic or cycloaliphatic alcohols, saturated or unsaturated fatty acids and alkylphenols, said derivatives contains 3 to 30 glycol ether groups and 8 to 20 carbon atoms in the (aliphatic) hydrocarbon moiety and 6 to 18 carbon atoms in the alkyl moiety of the alkylphenols. Further suitable non-ionic surfactants are the water-soluble adducts of polyethylene oxide with polypropylene glycol, ethylenediaminopolypropylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit. Representative examples of non-ionic surfactants are nonylphenolpolyethoxyethanols, castor oil polyglycol ethers, polypropylene/polyethylene oxide adducts, tributylphenoxy-polyethoxyethanol, polyethylene glycol and octylphenoxypolyethoxyethanol. Fatty acid esters of polyoxyethylene sorbitan, e.g. polyoxyethylene sorbitan trioleate, are also suitable.

Cationic surfactants are preferably quaternary ammonium salts which contain, as N-substituent, at least one C₈-C₂₂ alkyl radical and, as further substituents, unsubstituted or halogenated lower alkyl, benzyl or hydroxy-lower alkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described e.g. in the following publications: “McCutcheon's Detergents and Emulsifiers Annual” MC Publishing Corp., Ridgewood, N.J., 1981. Stache, H., “Tensid-Taschenbuch”, Carl Hanser Verlag Munich/Vienna 1981.

The compositions usually contain 0.1 to 99% by weight, preferably 0.1 to 95% by weight, of a compound X or M, 1 to 99.9% by weight, preferably 5 to 99.8% by weight, of a solid or liquid adjuvant and 0 to 25% by weight, preferably 0.1 to 25% by weight, of a surfactant.

The compositions may also contain further ingredients such as stabilizers, antifoams, viscosity regulators, binders, tackifiers as well as fertilizers or other active ingredients for obtaining special effects.

Various methods and techniques are suitable for employing compound X or M or compositions containing them for treating plant cells or plants. Such method may include
i) Incorporation into liquid or solidified media or substrates utilized during transformation, regeneration or growth of plant cells, plant material or plants.
ii) Seed dressing
iii) Application by spraying (e.g. from a tank mixture utilizing a liquid formulation)

### I.1.1 Compound X

Preferably compound X is comprising a substance comprising a structure selected from the group of consisting of D-tryptophane, D-histidine, D-arginine, D-threonine, D-methionine, D-serine, and D-alanine, more preferably a structure selected from the group consisting of D-serine, and D-alanine. Most preferably compound X is comprising a substance comprising the structure of D-alanine.

Preferably compound X is comprising a substance selected from the group of consisting of D-tryptophane, D-histidine, D-arginine, D-threonine, D-methionine, D-serine, and D-alanine, more preferably selected from the group consisting of D-serine, and D-alanine. Most preferably compound X is comprising D-alanine.

The use of D-alanine has the advantage that racemic mixtures of D- and L-alanine can be applied without disturbing or detrimental effects of the L-enantiomer. Therefore, in an improved embodiment an racemic mixture of D/L-alanine is employed as compound X.

Furthermore, D-amino acid structure comprising herbicidal compounds may be employed as compound X. Such compounds are for example described in U.S. Pat. No. 5,059,239, and may include (but shall not be limited to) N-benzoyl-N-(3-chloro-4-fluorophenyl)-DL-alanine, N-benzoyl-N-(3-chloro-4-fluorophenyl)-DL-alanine methyl ester, N-benzoyl-N-(3-chloro-4-fluorophenyl)-DL-alanine ethyl ester, N-benzoyl-N-(3-chloro-4-fluorophenyl)-D-alanine, N-benzoyl-N-(3-chloro-4-fluorophenyl)-D-alanine methyl ester, or N-benzoyl-N-(3-chloro-4-fluorophenyl)-D-alanine isopropyl ester.

When applied via the cell culture medium (e.g., incorporated into agar-solidified MS media plates), D-alanine can be employed in concentrations of about 0.1 mM to about 100 mM, preferably about 0.3 mM to about 30 mM, more preferably about 1 mM to about 5 mM.

When applied via the cell culture medium (e.g., incorporated into agar-solidified MS media plates), D-serine can be employed in concentrations of about 0.1 to about 10 mM, preferably about 0.3 to 4 mM, more preferably about 0.5 mM to about 1.5 mM.

### I.1.2 Compound M

Preferably compound M is comprising a substance comprising a structure selected from the group of consisting of D-isoleucine, D-valine, D-asparagine, D-leucine, D-lysine, D-proline, and D-glutamine, more preferably a structure selected from the group consisting of D-isoleucine, and D-valine. Most preferably compound M is comprising a substance comprising the structure of D-isoleucine.

Preferably compound M is comprising a substance selected from the group of consisting of D-isoleucine, D-valine, D-asparagine, D-leucine, D-lysine, D-proline, and D-glutamine, more preferably selected from the group consisting of D-isoleucine, and D-valine. Most preferably compound M is comprising D-isoleucine.

When applied via the cell culture medium (e.g., incorporated into agar-solidified MS media plates), D-isoleucine can be employed in concentrations of about 0.1 mM to about 100 mM, preferably about 1 mM to about 50 mM, more preferably about 10 mM to about 30 mM.

When applied via the cell culture medium (e.g., incorporated into agar-solidified MS media plates), D-valine can be employed in concentrations of about 1 to about 100 mM, preferably about 5 to 50 mM, more preferably about 15 mM to about 30 mM.

When applied via the cell culture medium (e.g., incorporated into agar-solidified MS media plates), D-asparagine or D-glutamine can be employed in concentrations of about 0.5 to about 100 mM, preferably about 1 to 50 mM, more preferably about 3 mM to about 20 mM.

### I.1.3 Mode of Application

As described above, the selection can be done during any step of plant cell culture, regeneration or plant growth. Surprisingly, the D-amino acid compounds are able to exhibit their growth modulating properties not only during cell culture (e.g., when applied on isolated plant cells, shoots or plantlets) but also later when applied on plants via spraying. When applied via spraying, D-alanine may be applied in concentrations of about 5 to about 100 mM, preferably from about 10 to about 80 mM, more preferably from about 40 to about 60 mM. When applied via spraying, D-serine may be applied in concentrations of about 5 to about 80 mM, preferably from about 10 to about 60 mM, more preferably from about 20 to about 40 mM.

### II. THE MARKER EXCISION FEATURE OF THE INVENTION

It is one essential feature of the invention that the dual-function marker of the invention is specifically deleted after its use. Preferably, deletion of the first expression cassette encoding for said dual-function marker can be realized by various means known in the art, including but not limited to one or more of the following methods:
a) recombination induced by a sequence specific recombinase, wherein said first expression cassette is flanked by corresponding recombination sites in a way that recombination between said flanking recombination sites results in deletion of the sequences in-between from the genome (for specific embodiments see III.1 below),
b) homologous recombination between homology sequences A and A′ flanking said first expression cassette, preferably induced by a sequence-specific double-strand break between said homology sequences caused by a sequence specific endonuclease, wherein said homology sequences A and A′ have sufficient length and homology in order to ensure homologous recombination between A and A′, and having an orientation which—upon recombination between A and A′—will lead to excision of said first expression cassette from the genome of said plant (for specific embodiments see III.2 below).

Accordingly, for ensuring marker deletion/excision the expression cassette for the D-amino acid oxidase (the first expression construct) comprised in the DNA construct of the invention is flanked by sequences which allow for specific deletion of said expression cassette. Said sequences may be recombination sites for a sequence specific recombinase, which are placed in a way the recombination induced between said flanking recombination sites results in deletion of the said first expression cassette from the genome. There are various recombination sites and corresponding sequence specific recombinases known in the art (described herein below), which can be employed for the purpose of the invention.

In another preferred embodiment, deletion/excision of the dual-marker sequence is performed by intramolecular (preferably intrachromosomal) homologous recombination. Homologous recombination may occur spontaneous but is preferably induced by a sequence-specific double-strand break (e.g., between the homology sequences). The basic principals are disclosed in WO 03/004659. For this purpose the first expression construct (encoding for the dual-function marker) is flanked by homology sequences A and A′, wherein said homology sequences have sufficient length and homology in order to ensure homologous recombination between A and A′, and having an orientation which—upon recombination between A and A′—will lead to an excision of first expression cassette from the genome. Furthermore, the sequence flanked by said homology sequences further comprises at least one recognition sequence of at least 10 base pairs for the site-directed induction of DNA double-strand breaks by a sequence specific DNA double-strand break inducing enzyme, preferably a sequence-specific DNA-endonuclease, more preferably a homing-endonuclease, most preferably a endonuclease selected from the group consisting of I-SceI, I-CpaI, I-CpaII, I-CreI and I-ChuI or chimeras thereof with ligand-binding domains. Suitable endonuclease are described herein below.

### III.1 Recombination Sites and Recombinases of the Invention

Sequence specific recombinases and their corresponding recombination sites suitable within the present invention may include but are not limited to the Cre/lox system of the bacteriophage P1 (Dale E C and Ow D W (1991) Proc Natl Acad Sci USA 88:10558-10562; Russell S H et al. (1992) Mol Gene Genet 234: 49-59; Osborne B I et al. (1995) Plant J. 7, 687-701), the yeast FLP/FRT system (Kilby N J et al. (1995) Plant J 8:637-652; Lyznik L A et al. (1996) Nucleic Acids Res 24:3784-3789), the Mu phage Gin recombinase, the E. coli Pin recombinase or the R/RS system of the plasmid pSR1 (Onouchi H et al. (1995) Mol Gen Genet 247:653-660; Sugita Ket al. (2000) Plant J. 22:461-469). The recombinase (for example Cre or FLP) interacts specifically with its corresponding recombination sequences (34 bp lox sequence and 47 bp FRT sequence, respectively) in order to delete or invert the interposed sequences. Deletion of standard selection marker in plants which was flanked by two lox sequences by the Cre is described (Dale E C and Ow D W (1991) Proc Natl Acad Sci USA 88:10558-10562). The preferred recombination sites for suitable recombinases are described in Table 4 below:

Organism

| | | |
|---|---|---|
| Recombinase | of origin | Recombination Sites |

| | | |
|---|---|---|
| CRE | Bacteriophage | 5′-AACTCTCATCGCTTCGGATAACT |
| | P1 | TCCTGTTATCCGAAACATATCACTCA |
| | | CTTTGGTGATTTCACCGTAACT-GTC |
| | | TATGATTAATG-3′ |
| FLP | Saccharomyces | 5′-GAAGTTCCTATTCCGAAGTTCCT |
| | cerevisiae | ATTCTCTAGAA AG-TATAGGAACTT |
| | | C-3′ |
| R | pSR1 | 5′-CGAGATCATATCACTGTGGACGT |
| | Plasmids | TGATGAAAGAATACGTTATTCTTTCA |
| | | TCAAATCGT |

Tab 4:
Suitable sequence specific recombinases for use in the method of the invention.

### III.2 The Homology Sequences

Referring to the homology sequences (e.g., A, A′) “sufficient length” preferably refers to sequences with a length of at least 20 base pairs, preferably at least 50 base pairs, especially preferably at least 100 base pairs, very especially preferably at least 250 base pairs, most preferably at least 500 base pairs.

Referring to the homology sequences (e.g., A, A′), “sufficient homology” preferably refers to sequences with at least 70%, preferably 80%, by preference at least 90%, especially preferably at least 95%, very especially preferably at least 99%, most preferably 100%, homology within these homology sequences over a length of at least 20 base pairs, preferably at least 50 base pairs, especially preferably at least 100 base pairs, very especially preferably at least 250 base pairs, most preferably at least 500 base pairs.

The homology sequences A and A′ are preferably organized in the form of a direct repeat. The term “direct repeat” means a subsequent localization of two sequences on the same strand of a DNA molecule in the same orientation, wherein these two sequences fulfill the above given requirements for homologous recombination between said two sequences.

In a preferred embodiment, the homology sequences may be a duplication of a sequence having additional use within the DNA construct. For example, the homology sequences may be two transcription terminator sequences. One of these terminator sequences may be operably linked to the agronomically valuable trait, while the other may be linked to the dual-function selection marker, which is localized in 3′-direction of the trait gene. Recombination between the two terminator sequences will excise the marker gene but will reconstitute the terminator of the trait gene. In another example, the homology sequences may be two promoter sequences. One of these promoter sequences may be operably linked to the agronomically valuable trait, while the other may be linked to the dual-function selection marker, which is localized in 5′-direction of the trait gene. Recombination between the two promoter sequences will excise the marker gene but will reconstitute the promoter of the trait gene. The person skilled in the art will know that the homology sequences do not need to be restricted to a single functional element (e.g. promoter or terminator), but may comprise or extent to other sequences (e.g. being part of the coding region of the trait gene and the respective terminator sequence of said trait gene.

### III.3. Double-Strand Break Inducing Enzyme of the Invention

Preferably, deletion/excision of the dual-function marker is realized by homologous recombination between the above specified homology sequences induced by a sequence-specific double-strand break, preferably between the homology sequences which should recombine. General methods are disclosed for example in WO 03/004659, incorporated herein entirely by reference. Various enzyme suitable for induction of sequence-specific double-strand breaks (hereinafter together “endonuclease”) are known in the art. The endonuclease may be for example selected from the group comprising:
1. Restriction endonucleases (type II), preferably homing endonucleases as described in detail hereinbelow.
2. Transposases, for example the P-element transposase (Kaufman P D and Rio D C (1992) Cell 69(1):27-39) or AcDs (Xiao Y L and Peterson T (2000) Mol Gen Genet 263(1):22-29). In principle, all transposases or integrases are suitable as long as they have sequence specificity (Haren L et al. (1999) Annu Rev Microbiol. 1999; 53:245-281; Beall E L, Rio D C (1997) Genes Dev. 11 (16):2137-2151).
3. Chimeric nucleases as described in detail hereinbelow.
4. Enzymes which induce double-strand breaks in the immune system, such as the RAG1/RAG2 system (Agrawal A et al. (1998) Nature 394(6695):744-451).
5. Group II intron endonucleases. Modifications of the intron sequence allows group II introns to be directed to virtually any sequence in a double-stranded DNA, where group II introns can subsequently insert by means of a reverse splice mechanism (Mohr et al. (2000) Genes & Development 14:559-573; Guo et al. (2000) Science 289:452-457). During this reverse splice mechanism, a double-strand break is introduced into the target DNA, the excised intron RNA cleaving the sense strand while the protein portion of the group II intron endonuclease hydrolyses the antisense strand (Guo et al. (1997) EMBO J. 16: 6835-6848). If it is only desired to induce the double-strand break without achieving complete reverse splicing, as is the case in the present invention, it is possible to resort to, for example, group II intron endonucleases which lack the reverse transcriptase activity. While this does not prevent the generation of the double-strand break, the reverse splicing mechanism cannot proceed to completion.

Suitable enzymes are not only natural enzymes, but also synthetic enzymes. Preferred enzymes are all those endonucleases whose recognition sequence is known and which can either be obtained in the form of their proteins (for example by purification) or expressed using their nucleic acid sequence.

In an preferred embodiment a sequence-specific endonuclease is employed for specific induction of double-strand breaks and subsequent induced homologous recombination. The term “Sequence specific DNA-endonuclease” generally refers to all those enzymes which are capable of generating double-strand breaks in double stranded DNA in a sequence-specific manner at one or more recognition sequences. Said DNA cleavage may result in blunt ends, or so called “sticky” ends of the DNA (having a 5′- or 3′-overhang). The cleavage site may be localized within or outside the recognition sequence. Various kinds of endonucleases can be employed. Endonucleases can be, for example, of the Class II or Class IIs type. Class IIs R-M restriction endonucleases catalyze the DNA cleavage at sequences other than the recognition sequence, i.e. they cleave at a DNA sequence at a particular number of nucleotides away from the recognition sequence (Szybalski et al. (1991) Gene 100:13-26). The following may be mentioned by way of example, but not by limitation:
1. Restriction endonucleases (e.g., type II or IIs), preferably homing endonucleases as described in detail hereinbelow.
2. Chimeric or synthetic nucleases as described in detail hereinbelow.

Unlike recombinases, restriction enzymes typically do not ligate DNA, but only cleave DNA. Restriction enzymes are described, for instance, in the New England Biolabs online catalog (www.neb.com), Promega online catalog (www.promega.com) and Rao et al. (2000) Prog Nucleic Acid Res Mol Biol 64:1-63. Within this invention “ligation” of the DNA ends resulting from the cleavage by the endonuclease is realized by fusion by homologous recombination of the homology sequences.

Preferably, the endonuclease is chosen in a way that its corresponding recognition sequences are rarely, if ever, found in the unmodified genome of the target plant organism. Ideally, the only copy (or copies) of the recognition sequence in the genome is (or are) the one(s) introduced by the DNA construct of the invention, thereby eliminating the chance that other DNA in the genome is excised or rearranged when the sequence-specific endonuclease is expressed.

One criterion for selecting a suitable endonuclease is the length of its corresponding recognition sequence. Said recognition sequence has an appropriate length to allow for rare cleavage, more preferably cleavage only at the recognition sequence(s) comprised in the DNA construct of the invention. One factor determining the minimum length of said recognition sequence is—from a statistical point of view—the size of the genome of the host organism. In an preferred embodiment the recognition sequence has a length of at least 10 base pairs, preferably at least 14 base pairs, more preferably at least 16 base pairs, especially preferably at least 18 base pairs, most preferably at least 20 base pairs.

A restriction enzyme that cleaves a 10 base pair recognition sequence is described in Huang B et al. (1996) J Protein Chem 15(5):481-9.

Suitable enzymes are not only natural enzymes, but also synthetic enzymes. Preferred enzymes are all those sequence specific DNA-endonucleases whose recognition sequence is known and which can either be obtained in the form of their proteins (for example by purification) or expressed using their nucleic acid sequence.

Especially preferred are restriction endonucleases (restriction enzymes) which have no or only a few recognition sequences—besides the recognition sequences present in the transgenic recombination construct—in the chromosomal DNA sequence of a particular eukaryotic organism. This avoids further double-strand breaks at undesired loci in the genome. This is why homing endonucleases are very especially preferred (Review: (Belfort M and Roberts R J (1997) Nucleic Acids Res 25: 3379-3388; Jasin M (1996) Trends Genet. 12:224-228; Internet: http://rebase.neb.com/rebase/re-base.homing.html). Owing to their long recognition sequences, they have no, or only a few, further recognition sequences in the chromosomal DNA of eukaryotic organisms in most cases.

The sequences encoding for such homing endonucleases can be isolated for example from the chloroplast genome of Chlamydomonas (Turmel M et al. (1993) J Mol Biol 232: 446-467). They are small (18 to 26 kD) and their open reading frames (ORF) have a “codon usage” which is suitable directly for nuclear expression in eukaryotes (Monnat R J Jr et al. (1999) Biochem Biophys Res Com 255:88-93). Homing endonucleases which are very especially preferably isolated are the homing endonucleases I-SceI (WO96/14408), I-SceII (Sarguiel B et al. (1990) Nucleic Acids Res 18:5659-5665), I-SceIII (Sarguiel B et al. (1991) Mol Gen Genet. 255:340-341), I-CeuI (Marshall (1991) Gene 104:241-245), I-CreI (Wang J et al. (1997) Nucleic Acids Res 25: 3767-3776), I-Chul (Cote V et al. (1993) Gene 129:69-76), I-TevI (Chu et al. (1990) Proc Natl Acad Sci USA 87:3574-3578; Bell-Pedersen et al. (1990) Nucleic Acids Res 18:3763-3770), I-TevII (Bell-Pedersen et al. (1990) Nucleic Acids Res 18:3763-3770), I-TevIII (Eddy et al. (1991) Genes Dev. 5:1032-1041), Endo SceI (Kawasaki et al. (1991) J Biol Chem 266:5342-5347), I-CpaI (Turmel M et al. (1995a) Nucleic Acids Res 23:2519-2525) and I-CpaII (Turmel M et al. (1995b) Mol. Biol. Evol. 12, 533-545).

Further homing endonucleases are detailed in the abovementioned Internet website, and examples which may be mentioned are homing endonucleases such as F-SceI, F-SceII, F-SuvI, F-TevI, F-TevII, I-AmaI, I-AniI, I-CeuI, I-CeuAIIP, I-ChuI, I-CmoeI, I-CpaI, I-CpaII, I-CreI, I-CrepsbIP, I-CrepsbIIP, I-CrepsbIIIP, I-CrepsbIVP, I-CsmI, I-CvuI, I-CvuAIP, I-DdiI, I-DdiII, I-DirI, I-DmoI, I-HmuI, I-HmuII, I-HspNIP, I-LlaI, I-MsoI, I-NaaI, I-NanI, I-NcIIP, I-NgrIP, I-NitI, I-NjaI, I-Nsp2361P, I-PakI, I-PboIP, I-PcuIP, I-PcuAI, I-PcuVI, I-PgrIP, I-PobIP, I-PorI, I-PorIIP, I-PpbIP, I-PpoI, I-SPBetaIP, I-ScaI, I-SceI, I-SceII, I-SceIII, I-SceIV, I-SceV, I-SceVI, I-SceVII, I-SexIP, I-SneIP, I-SpomCP, I-SpomIP, I-SpomIIP, I-SquIP, I-Ssp6803I, I-SthPhiJP, I-SthPhiST3P, I-SthPhiS3bP, I-TdeIP, I-TevI, 1-TevII, I-TevIII, I-UarAP, I-UarHGPA1P, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-PspI, PI-Rma438121P, PI-SPBetaIP, PI-SceI, PI-TfuI, PI-TfuII, PI-ThyI, PI-TliI, PI-TliII, H-DreI, I-BasI, I-BmoI, I-PogI, I-TwoI, PI-MgaI, PI-PabI, PI-PabII.

Preferred in this context are the homing endonucleases whose gene sequences are already known, such as, for example, F-SceI, I-CeuI, I-ChuI, I-DmoI, I-CpaI, I-CpaII, I-CreI, I-CsmI, F-TevI, F-TevII, I-TevI, I-TevII, I-AniI, I-CvuI, I-DdiI, 1-HmuI, 1-HmuII, I-LlaI, I-NanI, I-MsoI, I-NitI, I-NjaI, I-PakI, I-PorI, I-PpoI, I-ScaI, I-Ssp6803I, PI-PkoI, PI-PkoII, PI-PspI, PI-TfuI, PI-TliI. Especially preferred are commercially available homing endonucleases such as I-CeuI, I-SceI, I-DmoI, I-PpoI, PI-PspI or PI-SceI. Endonucleases with particularly long recognition sequences, and which therefore only rarely (if ever) cleave within a genome include: I-CeuI (26 bp recognition sequence), PI-PspI (30 bp recognition sequence), PI-SceI (39 bp recognition sequence), I-SceI (18 bp recognition sequence) and I-PpoI (15 bp recognition sequence). The enzymes can be isolated from their organisms of origin in the manner with which the skilled worker is familiar, and/or their coding nucleic acid sequence can be cloned. The sequences of various enzymes are deposited in GenBank. Very especially preferred are the homing endonucleases I-SceI, I-CpaI, I-CpaII, I-CreI and I-ChuI. Sequences encoding said nucleases are known in the art and—for example—specified in WO 03/004659 (e.g., as SEQ ID NO: 2, 4, 6, 8, and 10 of WP 03/004659 hereby incorporated by reference).

In an preferred embodiment, the sequences encoding said homing endonucleases can be modified by insertion of an intron sequence. This prevents expression of a functional enzyme in procaryotic host organisms and thereby facilitates cloning and transformations procedures (e.g., based on E. coli or Agrobacterium). In plant organisms, expression of a functional enzyme is realized, since plants are able to recognize and “splice” out introns. Preferably, introns are inserted in the homing endonucleases mentioned as preferred above (e.g., into I-SceI or I-CreI).

In some aspects of the invention, molecular evolution can be employed to create an improved endonuclease. Polynucleotides encoding a candidate endonuclease enzyme can, for example, be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer (1994) Proc Natl Acad Sci USA 91:10747-10751; Stemmer (1994) Nature 370:389-391; and U.S. Pat. No. 5,605,793, U.S. Pat. No. 5,837,458, U.S. Pat. No. 5,830,721 and U.S. Pat. No. 5,811,238.

Other synthetic endonucleases which may be mentioned by way of example are chimeric nucleases which are composed of an unspecific nuclease domain and a sequence-specific DNA binding domain consisting of zinc fingers (Bibikova M et al. (2001) Mol Cell Biol. 21:289-297). These DNA-binding zinc finger domains can be adapted to suit any DNA sequence. Suitable methods for preparing suitable zinc finger domains are described and known to the skilled worker (Beerli R R et al., Proc Natl Acad Sci USA. 2000; 97 (4):1495-1500; Beerli R R, et al., J Biol Chem 2000; 275(42):32617-32627; Segal D J and Barbas C F 3rd., Curr Opin Chem Biol 2000; 4(1):34-39; Kang J S and Kim J S, J Biol Chem 2000; 275(12):8742-8748; Beerli R R et al., Proc Natl Acad Sci USA 1998; 95(25):14628-14633; Kim J S et al., Proc Natl Acad Sci USA 1997; 94(8):3616-3620; Klug A, J Mol Biol 1999; 293(2):215-218; Tsai S Y et al., Adv Drug Deliv Rev 1998; 30(1-3):23-31; Mapp A K et al., Proc Natl Acad Sci USA 2000; 97(8):3930-3935; Sharrocks A D et al., Int J Biochem Cell Biol 1997; 29(12):1371-1387; Zhang L et al., J Biol Chem 2000; 275(43):33850-33860).

The endonuclease is preferably expressed as a fusion protein with a nuclear localization sequence (NLS). This NLS sequence enables facilitated transport into the nucleus and increases the efficacy of the recombination system. A variety of NLS sequences are known to the skilled worker and described, inter alia, by Jicks G R and Raikhel N V (1995) Annu. Rev. Cell Biol. 11:155-188. Preferred for plant organisms is, for example, the NLS sequence of the SV40 large antigen. Examples are provided in WO 03/060133. However, owing to the small size of many DSBI enzymes (such as, for example, the homing endonucleases), an NLS sequence is not necessarily required. These enzymes are capable of passing through the nuclear pores even without any aid.

In a further preferred embodiment, the activity of the endonuclease can be induced. Suitable methods have been described for sequence-specific recombinases (Angrand P O et al. (1998) Nucl. Acids Res. 26(13):3263-3269; Logie C and Stewart A F (1995) Proc Natl Acad Sci USA 92(13):5940-5944; Imai T et al. (2001) Proc Natl Acad Sci USA 98(1):224-228). These methods employ fusion proteins of the endonuclease and the ligand binding domain for steroid hormone receptor (for example the human androgen receptor, or mutated variants of the human estrogen receptor as described therein). Induction may be effected with ligands such as, for example, estradiol, dexamethasone, 4-hydroxytamoxifen or raloxifen. Some endonucleases are active as dimers (homo- or heterodimers; I-CreI forms a homodimer; I-SecIV forms a heterodimerk) (Wernette C M (1998) Biochemical & Biophysical Research Communications 248(1):127-333)). Dimerization can be designed as an inducible feature, for example by exchanging the natural dimerization domains for the binding domain of a low-molecular-weight ligand. Addition of a dimeric ligand then brings about dimerization of the fusion protein. Corresponding inducible dimerization methods, and the preparation of the dimeric ligands, have been described (Amara J F et al. (1997) Proc Natl Acad Sci USA 94(20): 10618-1623; Muthuswamy S K et al. (1999) Mol Cell Biol 19(10):6845-685; Schultz L W and Clardy J (1998) Bioorg Med Chem Lett. 8(1):1-6; Keenan T et al. (1998) Bioorg Med Chem. 6(8):1309-1335).

Recognition sequences for sequence specific DNA endonuclease (e.g., homing endonucleases) are described in the art. “Recognition sequence” refers to a DNA sequence that is recognized by a sequence-specific DNA endonuclease of the invention. The recognition sequence will typically be at least 10 base pairs long, is more usually 10 to 30 base pairs long, and in most embodiments, is less than 50 base pairs long.

“Recognition sequence” generally refers to those sequences which, under the conditions in a plant cell used within this invention, enable the recognition and cleavage by the sequence specific DNA-endonuclease. The recognition sequences for the respective sequence specific DNA-endonucleases are mentioned in Table 5 hereinbelow by way of example, but not by limitation.

TABLE 5

Recognition sequences and organisms of origin for
endonucleases (e.g., homing endonucleases “\hat{ }” indicates
the cleavage site of the sequence specific DNA-endonuclease
within a recognition sequence).

| | |
|---|---|
| DSBI | Organism |

| | | |
|---|---|---|
| Enzyme | of origin | Recognition sequence |

| | | |
|---|---|---|
| P- | Drosophila | 5′-CTAGATGAAATAACATAAGGTGG |
| Element | | |
| Trans- | | |
| posase | | |
| I-AniI | Aspergillus | 5′-TTGAGGAGGTT\hat{ }TCTCTGTAAATAANNNNNNNNNNNNNNN |
| | nidulans | 3′-AACTCCTCCAAAGAGACATTTATTNNNNNNNNNNNNNNN\hat{ } |
| I-DdiI | Dictyostelium | 5′-TTTTTTGGTCATCCAGAAGTATAT |
| | discoideumAX3 | 3′-AAAAAACCAG\hat{ }TAGGTCTTCATATA |
| I-CvuI | Chlorella | 5′-CTGGGTTCAAAACGTCGTGA\hat{ }GACAGTTTGG |
| | vulgaris | 3′-GACCCAAGTTTTGCAG\hat{ }CACTCTGTCAAACC |
| I-CsmI | Chlamydomonas | 5′-GTACTAGCATGGGGTCAAATGTCTTTCTGG |
| | smithii | |
| I-CmoeI | Chlamydomona- | 5′-TCGTAGCAGCT\hat{ }CACGGTT |
| | smoewusii | 3′-AGCATCG\hat{ }TCGAGTGCCAA |
| I-CreI | Chlamydomonas | 5′-CTGGGTTCAAAACGTCGTGA\hat{ }GACAGTTTGG |
| | reinhardtii | 3′-GACCCAAGTTTTGCAG\hat{ }CACTCTGTCAAACC |
| I-ChuI | Chlamydomonas | 5′-GAAGGTTTGGCACCTCG\hat{ }ATGTCGGCTCATC |
| | humicola | 3′-CTTCCAAACCGTG\hat{ }GAGCTACAGCCGAGTAG |
| I-CpaI | Chlamydomonas | 5′-CGATCCTAAGGTAGCGAA\hat{ }ATTCA |
| | pallidostigmatica | 3′-GCTAGGATTCCATC\hat{ }GCTTTAAGT |
| I-CpaII | Chlamydomonas | 5′-CCCGGCTAACTC\hat{ }TGTGCCAG |
| | pallidostigmatica | 3′-GGGCCGAT\hat{ }TGAGACACGGTC |
| I-CeuI | Chlamydomonas | 5′-CGTAACTATAACGGTCCTAA\hat{ }GGTAGCGAA |
| | eugametos | 3′-GCATTGATATTGCCAG\hat{ }GATTCCATCGCTT |
| I-DmoI | Desulfuro- | 5′-ATGCCTTGCCGGGTAA\hat{ }GTTCCGGCGCGCAT |
| | coccus mobilis | 3′-TACGGAACGGCC\hat{ }CATTCAAGGCCGCGCGTA |
| I-SceI | Saccharomyces | 5′-AGTTACGCTAGGGATAA\hat{ }CAGGGTAATATAG |
| | cerevisiae | 3′-TCAATGCGATCCC\hat{ }TATTGTCCCATTATATC |
| | | 5′-TAGGGATAA\hat{ }CAGGGTAAT |
| | | 3′-ATCCC\hat{ }TATTGTCCCATTA |
| | | (“Core”-Sequence) |
| I-SceII | S. cerevisiae | 5′-TTTTGATTCTTTGGTCACCC\hat{ }TGAAGTATA |
| | | 3′-AAAACTAAGAAACCAG\hat{ }TGGGACTTCATAT |
| I-SceIII | S. cerevisiae | 5′-ATTGGAGGTTTTGGTAAC\hat{ }TATTTATTACC |
| | | 3′-TAACCTCCAAAACC\hat{ }ATTGATAAATAATGG |
| I-SceIV | S. cerevisiae | 5′-TCTTTTCTCTTGATTA\hat{ }GCCCTAATCTACG |
| | | 3′-AGAAAAGAGAAC\hat{ }TAATCGGGATTAGATGC |
| I-SceV | S. cerevisiae | 5′-AATAATTTTCT\hat{ }TCTTAGTAATGCC |
| | | 3′-TTATTAAAAGAAGAATCATTA\hat{ }CGG |
| I-SceVI | S. cerevisiae | 5′-GTTATTTAATG\hat{ }TTTTAGTAGTTGG |
| | | 3′-CAATAAATTACAAAATCATCA\hat{ }ACC |
| I-SceVII | S. cerevisiae | 5′-TGTCACATTGAGGTGCACTAGTTATTAC |
| PI-SceI | S. cerevisiae | 5′-ATCTATGTCGGGTGC\hat{ }GGAGAAAGAGGTAAT |
| | | 3′-TAGATACAGCC\hat{ }CACGCCTCTTTCTCCATTA |
| F-SceI | S. cerevisiae | 5′-GATGCTGTAGGC\hat{ }ATAGGCTTGGTT |
| | | 3′-CTACGACA\hat{ }TCCGTATCCGAACCAA |
| F-SceII | S. cerevisiae | 5′-CTTTCCGCAACA\hat{ }GTAAAATT |
| | | 3′-GAAAGGCG\hat{ }TTGTCATTTTAA |
| I-HmuI | Bacillus subtilis | 5′-AGTAATGAGCCTAACGCTCAGCAA |
| | bacteriophage | 3′-TCATTACTCGGATTGC\hat{ }GAGTCGTT |
| | SPO1 | |
| I-HmuII | Bacillus subtilis | 5′-AGTAATGAGCCTAACGCTCAACAANNNNNNNNNNNNNNN |
| | bacteriophage | N-NNNNNNNNNNNNNNNNNNNNNNN |
| | SP82 | |
| I-LlaI | Lactococcus | 5′-CACATCCATAAC\hat{ }CATATCATTTTT |
| | lactis | 3′-GTGTAGGTATTGGTATAGTAA\hat{ }AAA |
| I-MsoI | Monomastix | 5′-CTGGGTTCAAAACGTCGTGA\hat{ }GACAGTTTGG |
| | species | 3′-GACCCAAGTTTTGCAG\hat{ }CACTCTGTCAAACC |
| I-NanI | Naegleria | 5′-AAGTCTGGTGCCA\hat{ }GCACCCGC |
| | andersoni | 3′-TTCAGACC\hat{ }ACGGTCGTGGGCG |
| I-NitI | Naegleria | 5′-AAGTCTGGTGCCA\hat{ }GCACCCGC |
| | italica | 3′-TTCAGACC\hat{ }ACGGTCGTGGGCG |
| I-NjaI | Naegleria | 5′-AAGTCTGGTGCCA\hat{ }GCACCCGC |
| | jamiesoni | 3′-TTCAGACC\hat{ }ACGGTCGTGGGCG |
| I-PakI | Pseudendoclonium | 5′-CTGGGTTCAAAACGTCGTGA\hat{ }GACAGTTTGG |
| | akinetum | 3′-GACCCAAGTTTTGCAG\hat{ }CACTCTGTCAAACC |
| I-PorI | Pyrobaculum | 5′-GCGAGCCCGTAAGGGT\hat{ }GTGTACGGG |
| | organotrophum | 3′-CGCTCGGGCATT\hat{ }CCCACACATGCCC |
| I-PpoI | Physarum | 5′-TAACTATGACTCTCTTAA\hat{ }GGTAGCCAAAT |
| | polycephalum | 3′-ATTGATACTGAGAG\hat{ }AATTCCATCGGTTTA |
| I-ScaI | Saccharomyces | 5′-TGTCACATTGAGGTGCACT\hat{ }AGTTATTAC |
| | capensis | 3′-ACAGTGTAACTCCAC\hat{ }GTGATCAATAATG |
| I- | Synechocystis | 5′-GTCGGGCT\hat{ }CATAACCCGAA |
| Ssp6803I | species | 3′-CAGCCCGAGTA\hat{ }TTGGGCTT |
| PI-PfuI | Pyrococcus | 5′-GAAGATGGGAGGAGGG\hat{ }ACCGGACTCAACTT |
| | furiosus Vc1 | 3′-CTTCTACCCTCC\hat{ }TCCCTGGCCTGAGTTGAA |
| PI-PfuII | Pyrococcus | 5′-ACGAATCCATGTGGAGA\hat{ }AGAGCCTCTATA |
| | furiosus Vc1 | 3′-TGCTTAGGTACAC\hat{ }CTCTTCTCGGAGATAT |
| PI-PkoI | Pyrococcus | 5′-GATTTTAGAT\hat{ }CCCTGTACC |
| | kodakaraensis | 3′-CTAAAA\hat{ }TCTAGGGACATGG |
| | KOD1 | |
| PI-PkoII | Pyrococcus | 5′-CAGTACTACG\hat{ }GTTAC |
| | kodakaraensis | 3′-GTCATG\hat{ }ATGCCAATG |
| | KOD1 | |
| PI-PspI | Pyrococcus sp. | 5′-AAAATCCTGGCAAACAGCTATTAT\hat{ }GGGTAT |
| | | 3′-TTTTAGGACCGTTTGTCGAT\hat{ }AATACCCATA |
| PI-TfuI | Thermococcus | 5′-TAGATTTTAGGT\hat{ }CGCTATATCCTTCC |
| | fumicolans | 3′-ATCTAAAA\hat{ }TCCAGCGATATAGGAAGG |
| | ST557 | |
| PI-TfuII | Thermococcus | 5′-TAYGCNGAYACN\hat{ }GACGGYTTYT |
| | fumicolans | 3′-ATRCGNCT\hat{ }RTGNCTGCCRAARA |
| | ST557 | |
| PI-ThyI | Thermococcus | 5′-TAYGCNGAYACN\hat{ }GACGGYTTYT |
| | hydrothermalis | 3′-ATRCGNCT\hat{ }RTGNCTGCCRAARA |
| PI-TliI | Thermococcus | 5′-TAYGCNGAYACNGACGG\hat{ }YTTYT |
| | litoralis | 3′-ATRCGNCTRTGNC\hat{ }TGCCRAARA |
| PI-TliII | Thermococcus | 5′-AAATTGCTTGCAAACAGCTATTACGGCTAT |
| | litoralis | |
| I-TevI | Bacteriophage | 5′-AGTGGTATCAAC\hat{ }GCTCAGTAGATG |
| | T4 | 3′-TCACCATAGT\hat{ }TGCGAGTCATCTAC |
| I-TevII | Bacteriophage | 5′-GCTTATGAGTATGAAGTGAACACGT\hat{ }TATTC |
| | T4 | 3′-CGAATACTCATACTTCACTTGTG\hat{ }CAATAAG |
| F-TevI | Bacteriophage | 5′-GAAACACAAGA\hat{ }AATGTTTAGTAAANNNNNNNNNNNN NN |
| | T4 | 3′-CTTTGTGTTCTTTACAAATCATTTNNNNNNNNNNNNNN\hat{ } |
| F-TevII | Bacteriophage | 5′-TTTAATCCTCGCTTC\hat{ }AGATATGGCAACTG |
| | T4 | 3′-AAATTAGGAGCGA\hat{ }AGTCTATACCGTTGAC |
| H-DreI | E. coli pl-DreI | 5′-CAAAACGTCGTAA\hat{ }GTTCCGGCGCG |
| | | 3′-GTTTTGCAG\hat{ }CATTCAAGGCCGCGC |
| I-BasI | Bacillus- | 5′-AGTAATGAGCCTAACGCTCAGCAA |
| | thuringiensis | 3′-TCATTACGAGTCGAACTCGGATTG |
| | phage | |
| | Bastille | |
| I-BmoI | Bacillus | 5′-GAGTAAGAGCCCG\hat{ }TAGTAATGACATGGC |
| | mojavensis | 3′-CTCATTCTCG\hat{ }GGCATCATTACTGTACCG |
| | s87-18 | |
| I-PogI | Pyrobaculum | 5′-CTTCAGTAT\hat{ }GCCCCGAAAC |
| | oguniense | 3′-GAAGT\hat{ }CATACGGGGCTTTG |
| I-TwoI | Staphylococcus | 5′-TCTTGCACCTACACAATCCA |
| | aureus phage | 3′-AGAACGTGGATGTGTTAGGT |
| | Twort | |
| PI-MgaI | Mycobacterium | 5′-CGTAGCTGCCCAGTATGAGTCA |
| | gastri | 3′-GCATCGACGGGTCATACTCAGT |
| PI-PabI | Pyrococcus | 5′-GGGGGCAGCCAGTGGTCCCGTT |
| | abyssi | 3′-CCCCCGTCGGTCACCAGGGCAA |
| PI-PabII | Pyrococcus | 5′-ACCCCTGTGGAGAGGAGCCCCTC |
| | abyssi | 3′-TGGGGACACCTCTCCTCGGGGAG |

Also encompassed are minor deviations (degenerations) of the recognition sequence which still enable recognition and cleavage by the sequence specific DNA-endonuclease in question. Such deviations—also in connection with different frame-work conditions such as, for example, calcium or magnesium concentration—have been described (Argast G M et al. (1998) J Mol Biol 280: 345-353). Also encompassed are core sequences of these recognition sequences and minor deviations (degenerations) in there. It is known that the inner portions of the recognition sequences suffice for an induced double-strand break and that the outer ones are not absolutely relevant, but can codetermine the cleavage efficacy. Thus, for example, an 18 bp core sequence can be defined for I-SceI.

### III.4. Combination with Other Recombination Enhancing Techniques

In a further preferred embodiment, the efficacy of the recombination system is increased by combination with systems which promote homologous recombination. Such systems are described and encompass, for example, the expression of proteins such as RecA or the treatment with PARP inhibitors. It has been demonstrated that the intrachromosomal homologous recombination in tobacco plants can be increased by using PARP inhibitors (Puchta H et al. (1995) Plant J. 7:203-210). Using these inhibitors, the homologous recombination rate in the DNA constructs of the invention after induction of the sequence-specific DNA double-strand break, and thus the efficacy of the deletion of the transgene sequences, can be increased further. Various PARP inhibitors may be employed for this purpose. Preferably encompassed are inhibitors such as 3-aminobenzamide, 8-hydroxy-2-methylquinazolin-4-one (NU1025), 1,11b-dihydro-[2H]benzopyrano[4,3,2-de]isoquinolin-3-one (GPI 6150), 5-aminoisoquino-linone, 3,4-dihydro-5-[4-(1-piperidinyl)butoxy]-1(2H)-isoquinolinone, or the compounds described in WO 00/26192, WO 00/29384, WO 00/32579, WO 00/64878, WO 00/68206, WO 00/67734, WO 01/23386 and WO 01/23390.

In addition, it was possible to increase the frequency of various homologous recombination reactions in plants by expressing the E. coli RecA gene (Reiss B et al. (1996) Proc Natl Acad Sci USA 93(7):3094-3098). Also, the presence of the protein shifts the ratio between homologous and illegitimate DSB repair in favor of homologous repair (Reiss B et al. (2000) Proc Natl Acad Sci USA 97(7):3358-3363). Reference may also be made to the methods described in WO 97/08331 for increasing the homologous recombination in plants. A further increase in the efficacy of the recombination system might be achieved by the simultaneous expression of the RecA gene or other genes which increase the homologous recombination efficacy (Shalev G et al. (1999) Proc Natl Acad Sci USA 96(13):7398-402). The above-stated systems for promoting homologous recombination can also be advantageously employed in cases where the DNA construct of the invention is to be introduced in a site-directed fashion into the genome of a eukaryotic organism by means of homologous recombination.

### III.5 Initiation of Deletion/Excision

There are various means to appropriately initiate deletion/excision of the dual-function marker. Preferably deletion is only initiated after the dual-function marker has successfully completed its function has negative selection marker resulting in insertion of the DNA construct of the invention into the genome of the cell or organism to be transformed.

Various means are available for the person skilled in art to combine the deletion/excision inducing mechanism with the DNA construct of the invention comprising the D-amino oxidase dual-function selection marker. Preferably, a recombinase or endonuclease (hereinafter together also “excision enzyme”) employable in the method of the invention can be expressed or combined with its corresponding recombination or recognition site, respectively, by a method selected from the group consisting of:
a) incorporation of a second expression cassette for expression of the excision enzyme (the recombinase or sequence-specific endonuclease) operably linked to a plant promoter into said DNA construct, preferably together with said first expression cassette flanked by said sequences which allow for specific deletion,
b) incorporation of a second expression cassette for expression of the excision enzyme (the recombinase or sequence-specific endonuclease) operably linked to a plant promoter into the plant cells or plants used as target material for the transformation thereby generating master cell lines or cells,
c) incorporation of a second expression cassette for expression of the excision enzyme (the recombinase or sequence-specific endonuclease) operably linked to a plant promoter into a separate DNA construct, which is transformed by way of co-transformation with said first DNA construct into said plant cells or transformed into cells already comprising said first DNA construct.

Accordingly the first DNA construct of the invention and the excision enzyme (e.g., the recombinase or endonuclease) can be combined in a plant organism, cell, cell compartment or tissue for example as follows:
1.) Plants which have the first DNA construct inserted into their genome (preferably into the chromosomal DNA) are generated in the customary manner utilizing the dual-function marker as negative selection marker (for example, by Agrobacteria mediated transformation). A second expression cassette for the excision enzyme is then combined with said DNA constructs by
   a) a second transformation with said second expression cassette, or
   b) crossing of the plants comprising the first DNA construct with master plants comprising the expression cassette for the excision enzyme.
2.) The expression cassette encoding for the excision enzyme can be integrated into the DNA construct which already bears the expression cassette for the dual-function marker. It is preferred to insert the sequence encoding the excision enzyme between the sequences allowing for deletion and thus to delete it from the genomic DNA after it has fulfilled its function. Very especially preferably, expression of the endonuclease is inducible in such a case (for example under the control of one of the inducible promoters described hereinbelow), in a development-dependent fashion using a development-dependent promoter, or else excision enzymes are employed whose activity is inducible in order to avoid premature deletion of the dual-function marker prior to its insertion into the genome.
4.) Relying on the co-transformation technique, the expression cassette which ensures the expression of the excision enzyme can be transformed into the cells simultaneously with the first DNA construct, but on a separate vector. Co-transformation can be in each case stable or transient. In such a case, expression of the excision enzyme is preferably inducible (for example under the control of one of the inducible promoters described hereinbelow), in a development-dependent fashion using a development-dependent promoter, or else excision enzymes are employed whose activity is inducible in order to avoid premature deletion of the dual-function marker prior to its insertion into the genome.
5.) Plants expressing the excision enzyme may also act as parent individuals. In the progeny from the crossing between plants expressing the excision enzyme on the one hand and plants bearing the first DNA construct on the other hand, the desired marker deletion (e.g., by double-strand breaks and recombination between the homology sequences) are observed.
6.) Expression of the excision enzyme is also conceivable in a transient transformation approach in which the possibilities 2 to 4 can be exploited.
7.) The excision enzyme can also be introduced into cells comprising or bearing the transgenic recombination construct directly, for example via microinjection, particle bombardment (biolistic method), polyethylene glycol transfection or liposome-mediated transfection. This embodiment is advantageous since no excision enzyme-encoding sequences remains in the genome. Such a method has been described for example by Segal D J et al. (1995) Proc Natl Acad Sci USA 92:806-810.
8.) The excision enzyme may also be generated by introducing the excision enzyme-encoding, in-vitro-generated mRNA into cells (for example via microinjection, particle bombardment (biolistic method) or liposome-mediated transfection). This embodiment is advantageous since no excision-enzyme-encoding sequences will remain in the genome.
9.) The excision enzyme can be introduced into plant cells as a fusion protein with the VirE2 or VirF protein of an Agrobacterium. Such methods have been described for example for Cre recombinase (Vergunst A C et al. (2000) Science. 290: 979-982). If the expression cassette for the fusion protein is located outside the border sequences, it is not inserted

As described above, the excision enzyme can be generated using an expression cassette which comprises the DNA encoding an excision enzyme and is introduced into a plant cell or organism. In this context, the expression cassette for the excision enzyme preferably comprises a nucleic acid sequence encoding an excision enzyme. Various suitable cassettes are described in WO 03/004659.

A preferred embodiment of the invention is related to DNA constructs comprising both the expression cassette for the dual-function marker (the first expression cassette) and a second expression cassette for the excision enzyme (e.g., an endonuclease or recombinase encoding sequence linked to a plant promoter), preferably in a way that said second expression cassette is together with said first expression cassette flanked by said sequences which allow for specific deletion.

In another preferred embodiment the mechanism of deletion/excision can be induced or activated in a way to prevent pre-mature deletion/excision of the dual-function marker. Preferably, thus expression and/or activity of an preferably employed excision enzyme can be induced, preferably by a method selected from the group consisting of
a) inducible expression by operably linking the sequence encoding said excision enzyme (e.g., a recombinase or endonuclease) to an inducible promoter,
b) inducible activation, by employing a modified excision enzyme (e.g., a recombinase or endonuclease) comprising a ligand-binding-domain, wherein activity of said modified excision enzyme can by modified by treatment of a compound having binding activity to said ligand-binding-domain.

Expression of the polynucleotide encoding the excision enzyme is preferably controlled by an excision promoter, which allows for expression in a timely manner so that the dual-function marker can perform its function as a negative selection marker before getting excised. Suitable promoters are for example described in the German Patent Application DE 03028884.9. Such promoters may have for example expression specificity for late developmental stages like e.g., reproductive tissue. The excision promoter may be selected from one of the following groups of promoters:
a) Pollen-specific promoters such as, for example, the promoter of the B. campestris bgp1 gene (GenBank Acc.-No: X68210; Xu H et al. (1993) Mol Gen Genet 239(1-2):58-65; WO 94/13809), of the Oryza sativa ory s 1 gene (GenBank Acc.-No.: AJ012760; Xu H et al. (1995) Gene 164 (2):255-259), of the pollen-specific maize gene ZM13 (Hamilton D A et al. (1998) Plant Mol Biol 38(4):663-669; U.S. Pat. No. 5,086,169), and of the B. napus gene Bp10 (GenBank Acc.-No.: X64257; Albani D (1992) Plant J 2(3):331-342; U.S. Pat. No. 6,013,859). The promoter of the potato invGF gene from potato (Plant Mol. Biol., 1999, 41:741-751; EMBL Acc No. AJ133765; especially preferred is the promoter described in German Patent Application DE 03028884.9 by SEQ ID NO: 1). The Lcg1 promoter (WO 99/05281; XU H et al. (1999) Proc. Natl. Acad. Sci. USA Vol. 96:2554-2558).
b) Promoters active in ovules (i.e. egg cells)
   The promoter of the Arabidopsis AtSERK1 gene (Somatic Embryogenesis Receptor-Like Kinase 1; At1G71830; Hecht et al. (2001) Plant Physiol 127:803-816). Especially preferred is the promoter described in German Patent Application DE 03028884.9 by SEQ ID NO: 2.
c) Promoters active in zygotes
   The promoter of the Arabidopsis gene Atcyc1A (Cyclin cyc1 gene, type cyclin B; At4g37490; Plant Cell 6: 1763-1774 (1994)). Especially preferred is the promoter described in German Patent Application DE 03028884.9 by SEQ ID NO: 5. USP promoter from Vicia faba (Bäumlein H et al. (1991) Mol Gen Genet 225:459-467; Fiedler U et al. (1993) Plant Mol Biol 22:669-679). Especially preferred is the promoter described in German Patent Application DE 03028884.9 by SEQ ID NO: 3. The USP promoter has further activity also in early immature embryos.
d) Promoters active in meristems

The promoter of the gene erecta (Acc. No. D83257) from Arabidopsis is active in meristematic cells (Yokoyama et al., 1998, Plant J. 15: 301-310). Especially preferred is the promoter described in German Patent Application DE 03028884.9 by SEQ ID NO: 4.

Alternatively, an inducible promoter (which may have ubiquitous expression activity when induced) can be employed with the application of the corresponding inducer at the appropriate time point. Preferably, the inducer for the inducible promoter is applied together with (or briefly before) application of the compound M (e.g., D-isoleucine or D-valine) which is converted by action of the dual-function DAAO marker into a phytotoxic compound.

The term “inducible” as applied to a promoter is well understood by those skilled in the art. In essence, expression under the control of an inducible promoter is “switched on” or increased in response to an applied stimulus (which may be generated within a cell or provided exogenously). The nature of the stimulus varies between promoters. Whatever the level of expression is in the absence of the stimulus, expression from any inducible promoter is increased in the presence of the correct stimulus. The preferable situation is where the level of expression increases upon in the presence of the relevant stimulus by an amount effective to alter a phenotypic characteristic (i.e. to express a DAAO and modify tolerance of a D-amino acid). Thus an inducible (or “switchable”) promoter may be used which causes a basic level of expression in the absence of the stimulus which level is too low to bring about the desired D-amino acid tolerant or sensitive phenotype (and may in fact be zero). Upon application of the stimulus, expression is increased (or switched on) to a level that causes enhanced D-amino acid tolerance or sensitivity. Many examples of inducible promoters will be known to those skilled in the art.

The inducer can be a physical stimulus like light, heat, drought (low moisture), wounding etc. However, preferably, the inducer is an externally applied chemical substance. It is preferred that the inducible excision promoter only causes functional expression of the endonuclease operably linked if this chemical inducer is externally applied. This leads to a controlled, governable expression and deletion.

Inducible and repressible promoters have been developed for use in plants (Rewiew: Gatz, Annu Rev Plant Physiol Plant Mol Biol 1997, 48:89-108), based on—for example—bacterial repressor (Gatz C & Quail P H (1988) Proc. Natl. Acad. Sci. USA 85:1394-1397), animal steroid (Aoyarna T & Chua N H (1997) Plant J. 11:605-612; Martinez A et al. (1999) Plant J. 19:97-106) or fungal regulatory elements (Caddick M X et al. (1998) Nature Biotechnol 16:177-180). Promoter systems that are positively regulated by chemical ligands (inducible systems) include the tetracycline(doxycycline)-induced ‘Triple-Op’ promoter (Gatz C & Quail P H (1988) Proc Natl Acad Sci USA 85:1394-1397; Gatz C et al. (1991) Mol Gen Genet 277:229-237; Gatz C et al. (1992) Plant J. 2:397-404), the glucocorticoid-inducible ‘GAL4-UAS’ promoter (Aoyarna T & Chua N H (1997) Plant J. 11:605-612), the ecdysone-inducible ‘GRHEcR’ promoter (Martinez A et al. (1999) Plant J. 19:97-106) and the ethanol-inducible ′alcA promoter (Caddick M X et al. (1998) Nature Biotechnol 16:177-180). Hormones that have been used to regulate gene expression include, for example, estrogen, tomoxifen, toremifen and ecdysone (Ramkumar and Adler (1995) Endocrinology 136: 536-542). See, also, Gossen and Bujard Proc. Natl. Acad. Sci. USA 89: 5547 (1992); Gossen et al. Science 268: 1766 (1995). In tetracycline-inducible systems, tetracycline or doxycycline modulates the binding of a repressor to the promoter, thereby modulating expression from the promoter.

Inducible expression system can be distinguished into positively and negatively regulated systems. For positively regulated system, expression is induced by adding the corresponding inducer, for negatively regulated systems expression is induced by removing the inducer (better named repressor in this case). An example for a negatively regulated (repressible) system is the tetracycline-inactivated ‘Top10’ promoter and derivatives (Bohner S et al. (1999) Plant J. 19. 87-95; Weinmann P et al. (1994) Plant J 5:559-569). The Top10 promoter sequence contains a tandem repeat of seven copies of the Tn10 tet operator (tet-OP) DNA sequence that tightly bind the tetracycline repressor polypeptide TetR (Lederer T et al. (1995) Anal Biochem 232:190-196). This element is fused to a truncated version of e.g., the CaMV 35S promoter (nucleotide positions −53 to 0). The Top10 promoter sequence is recognized by a transactivator that effectively acts as an artificial transcription factor. The transactivator is a chimeric protein fusion between amino acids 1-207 of TetR (Postle K et al. (1984) Nucl Acids Res 12:4849-4963) and amino acids 363-490 of the transcriptional activation domain (VP16) from the Herpes simplex virus (Triezenberg S J et al. (1988) Genes Dev. 2:718-729), and is labelled ‘TetR/VP16’ or ′tTA (tetracycline transactivator). In the absence of tetracycline, the TetR portion of the tTA binds the tet-OP DNA sequences within the Top10 promoter with high affinity (Hinrichs W et al. (1994) Science 264:418-420; Lederer T et al. (1995) Anal Biochem 232:190-196; Lederer T et al. (1996) Biochemistry 35:7439-7446). This interaction positions the VP16 domain of the tTA in close proximity to the Top10 promoter TATA box, enabling transgene transcription. However, in the presence of tetracycline, the TetR undergoes a conformational change (Hinrichs W et al. (1994) Science 264:418-420; Orth P et al. (1998) J Mol Biol 279: 439-447) that lowers its affinity for the Top10 promoter to non-specific binding levels (Lederer T et al. (1996) Biochemistry 35:7439-7446). Consequently, tTA binding to the Top10 promoter is inhibited, and transcription is switched off. Use of the Top10 promoter system is particularly advantageous in plants. First the Top10 promoter is not functional in the absence of the tTA. Second, transcriptional control is stringent, and tightly controlled by tetracycline. Third, tetracycline has no naturally occurring analogue in plant cells, which might otherwise interfere with promoter regulation. Fourth, the levels of tetracycline used to repress the Top10 promoter are extremely low, normally of the order of 1 μg/ml, and have no discernible secondary effect on plants (Weinmann P et al. (1994) Plant J 5:559-569). Finally, coupling the two transformations required for promoter function can be achieved by transforming the same plants first with the 35S::tTA plasmid construct and then with the Top10 promoter driving the gene of interest, or by mating transgenics which have independently been transformed with the appropriate constructs. The Top10 promoter has been successfully used in Nicotiana sp. (Weinmann P et al. (1994) Plant J 5:559-569) and in the moss Physcomitrella patens (Zeidler M et al. (1996) Plant Mol Biol 30:199-205).

Alternatively, a positively regulated tetracyclin based inducible expression system can be employed. Especially preferred is the inducible reverse tetracycline system, which allows expression to be up-regulated only upon addition of tetracyclin or a lipid-soluble derivative of tetracycline, doxycyclin (dox, Gossen M. et al. (1995) Science 268:1766-1769; Jiang D M et al. (2001) J. Neurochem. 76(6); 1745-1755).

Inducible promoters that are directly responsive to physiologically active stimuli such as heat-shock (Prandl R et al. (1995) Plant Mol. Biol. 28:73-82; 1995; Severin K & Schoeffl F (1990) Plant Mol. Biol. 15:827-834), stress signalling molecules (Suchara K I et al. (1996) J. Ferm. Bioeng. 82, 51-55) or heavy metals (McKenzie, M. J., et al. (1998) Plant Physiol. 116, 969-977) may also be employed. However, chemically inducible promoter systems are preferred.

Inducibel expression systems have been used in several plant species, including tobacco (Gatz C et al. (1991) Mol. Gen. Genet. 277:229-237), potato (Kumar A et al. (1996) Plant J. 9:147-158), tomato (Thompson A J & Myatt S C (1997) Plant Mol. Biol. 34:687-692) and Arabidopsis thaliana (Aoyarna T & Chua N H (1997) Plant J. 11:605-612).

An additional example includes the ecdysone responsive element (No et al., Proc. Natl. Acad. Sci. USA 93: 3346 (1997)). Other examples of inducible promoters include the glutathione-S-transferase II promoter which is specifically induced upon treatment with chemical safeners such as N,N-diallyl-2,2-dichloroacetamide (PCT Application Nos. WO 90/08826 and WO 93/01294) and the alcA promoter from Aspergillus, which in the presence of the alcR gene product is induced with cyclohexanone (Lockington, et al., Gene 33: 137-149 (1985); Felenbok, et al. Gene 73: 385-396 (1988); Gwynne, et al. Gene 51: 205-216 (1987)) as well as ethanol. Chemical inducers of promoters can be combined with other active chemicals or inert carriers prior to application to an organism. For example, other agronomically useful chemical compositions such as pesticides or fertilizers as well as carriers and solvents can be combined with the inducer.

Further examples for inducible promoters include the PRP1 promoter (Ward et al., Plant. Mol. Biol. 22 (1993), 361-366), a salicylic-acid-inducible promoter (WO 95/19443), a benzenesulfonamide-inducible promoter (EP-A-0388186), a tetracyclin-inducible promoter (Gatz et al., (1992) Plant J. 2, 397-404), an abscisic acid-inducible promoter (EP-A 335528), a salicylic acid-inducible promoter (WO 95/19443) or an ethanol—(Salter M G et al. (1998) Plant J. 16:127-132) or cyclohexanone-inducible (WO 93/21334) promoter may likewise be used.

Other preferred promoters are promoters induced by biotic or abiotic stress, such as, for example, the pathogen-inducible promoter of the PRP1 gene (Ward et al., Plant Mol Biol 1993, 22: 361-366), the tomato heat-inducible hsp80 promoter (U.S. Pat. No. 5,187,267), the potato chill-inducible alpha-amylase promoter (WO 96/12814) or the wound-induced pinII promoter (EP375091).

In an especially preferred embodiment, the excision enzyme is expressed under the control of an inducible promoter. This leads to a controlled, governable expression and deletion—for example in plants—, and any problems caused by a constitutive expression of an excision enzyme are avoided.

Obviously, also the promoter controlling expression of the agronomically valuable trait or selection marker gene may be selected from the promoters preferred as excision promoters.

### III.6 Optional Methods of Preventing Premature Excision of the Excision Construct

It is useful to have a system to maintain the dual-function marker comprising construct of the invention especially during transformation and selection. In general, a control polynucleotide can be introduced into the DNA-construct encoding for the excision enzyme to achieve this goal. The control polynucleotide generally functions either to inhibit expression of the excision enzyme when inhibition is desired (e.g., during trans-formation and selection; for preferred time frames see above) or to release repression of the excision promoter, thus allowing for expression from the excision promoter. Those of skill will recognize that there are numerous variations for controlling or pre-venting expression of the excision enzyme in a particular cell or tissue or at a particular developmental stage.

In one aspect, expression from the first excision promoter (i.e. the promoter operably linked to the a first excision enzyme, which excises the dual-function marker) can be countered by a second no-excision promoter. For example, the second no-excision promoter can be operably linked to a repressor gene, which, when expressed, prevents expression of the first excision promoter. Examples of repressors include the tet and lac repressors (Gatz, et al. (1991) Mol Gen Genet. 227:229-237). The second no-excision promoter is preferably a promoter which has the highest activity in the tissue used for transformation/selection but has low activity in the reproductive cell (e.g., pollen or oocyte), a precursor cell or tissue of said reproductive cell, or an omnipotent cell (e.g. zygote) resulting from reproduction. Also an inducible promoter can be employed and induction is used during the transformation/selection phase. Such an inducible promoter can be for example a tetracycline (doxycycline)-inducible system, which is induced by tetracycline or doxycycline (see above). Antibiotics like this can be employed during transformation/selection.

Alternatively, the second no-excision promoter can be linked to the polynucleotide encoding the endonuclease in the opposite orientation of the first excision promoter (i.e., from the 3′-end of the coding sequence towards the 5′-end of the sequence), thereby interrupting expression of the DNA cleaving enzyme. In these embodiments, the transcriptional activity of the second no-excision promoter prevents completion of transcripts from the first excision promoter, thereby preventing expression of the excision enzyme.

In other embodiments, an antisense polynucleotide or a polynucleotide producing a double-stranded RNA molecule can be operably linked to the second no-excision promoter, thereby preventing the translation of the DNA cleaving enzyme mRNA. See, e.g., Sheehy et al. (1988) Proc Natl Acad Sci USA 85:8805-8809, and U.S. Pat. No. 4,801,340 for a description of antisense technology; and EP-A1 1 042 462, EP-A1 1 068 311 for a description of the double-stranded RNA interference technique. The antisense or double-stranded RNA molecule should have homology to the nucleotide encoding the excision enzyme to guarantee efficient suppression. In general, antisense technology involves the generation of RNA transcripts that hybridize to a target transcript (i.e., the transcript encoding the sequence-specific endonuclease). Alternatively, the second no-excision promoter can be operably linked to a DNA cleaving enzyme polynucleotide in the sense orientation to induce sense suppression of the gene (see, e.g., Napoli et al. (1990) Plant Cell 2:279-289, U.S. Pat. No. 5,034,323, U.S. Pat. No. 5,231,020, and U.S. Pat. No. 5,283,184 for a description of sense suppression technology).

In some embodiments, aptamer technology can be used to repress expression of the first excision promoter. See, e.g., Hermann et al. (2000) Science 287(5454):820-5; and Famulok et al. (1999) Curr Top Microbiol Immunol 243:123-36. For example, a small oligonucleotide could be developed that only binds and represses the first excision promoter when stabilized by a particular chemical which can be applied when transgenic seed are desired. For example, combinatorial library selections through the systematic evolution of ligands by exponential enrichment (SELEX) technique can be used to identify nucleic acid aptamers that bind with high affinity and specificity to a wide range of selected molecules. See, e.g., Conrad et al. (1995) Mol Divers 1(1):69-78; and Kusser (2000) J Biotechnol 74(I):27-38.

In some embodiments, a multi-tiered excision system is used. For example, the first excision promoter can be interrupted by a second recombination cassette. This second recombination cassette may again be flanked by a second set of homology sequences B and B′ flanking a chemically-induced promoter operably linked to a polynucleotide encoding a second sequence-specific DNA cleaving enzyme. In general, this system allows for the transgenic construct to remain intact in the genome (e.g., during trans-formation and selection) as long as the chemical inducer is not provided. Once the chemical inducer is presented, the second DNA cleaving enzyme is induced and excises its own coding region, induces homologous recombination between B and B′, thereby reconstituting the first excision promoter to an intact promoter. Since B remains after excision, B and B′ are preferably a sub-sequence of said first excision promoter.

### IV. ADDITIONAL ELEMENTS IN THE DNA CONSTRUCTS OF THE INVENTION

The DNA construct may—beside the various promoter sequences—comprise additional genetic control sequences. The term “genetic control sequences” is to be understood in the broad sense and refers to all those sequences which affect the making or function of the DNA construct to the invention or an expression cassette comprised therein. Preferably, an expression cassettes according to the invention encompass 5′-upstream of the respective nucleic acid sequence to be expressed a promoter and 3′-downstream a terminator sequence as additional genetic control sequence, and, if appropriate, further customary regulatory elements, in each case in operable linkage with the nucleic acid sequence to be expressed.

Genetic control sequences are described, for example, in “Goeddel; Gene Expression Technology: Methods in Enzymology 185, Academic Press, San Diego, Calif. (1990)” or “Gruber and Crosby, in: Methods in Plant Molecular Biology and Biotechnolgy, CRC Press, Boca Raton, Fla., eds.: Glick and Thompson, Chapter 7, 89-108” and the references cited therein.

Examples of such control sequences are sequences to which inductors or repressors bind and thus regulate the expression of the nucleic acid. Genetic control sequences furthermore also encompass the 5′-untranslated region, introns or the non-coding 3′-region of genes. It has been demonstrated that they may play a significant role in the regulation of gene expression. Thus, it has been demonstrated that 5′-untranslated sequences are capable of enhancing the transient expression of heterologous genes. Furthermore, they may promote tissue specificity (Rouster J et al. (1998) Plant J 15:435-440.). Conversely, the 5′-untranslated region of the opaque-2 gene suppresses expression. Deletion of the region in question leads to an increased gene activity (Lohmer S et al. (1993) Plant Cell 5:65-73). Genetic control sequences may also encompass ribosome binding sequences for initiating translation. This is preferred in particular when the nucleic acid sequence to be expressed does not provide suitable sequences or when they are not compatible with the expression system.

The expression cassette can advantageously comprise one or more of what are known as enhancer sequences in operable linkage with the promoter, which enable the increased transgenic expression of the nucleic acid sequence. Additional advantageous sequences, such as further regulatory elements or terminators, may also be inserted at the 3′ end of the nucleic acid sequences to be expressed recombinantly. One or more copies of the nucleic acid sequences to be expressed recombinantly may be present in the gene construct. Genetic control sequences are furthermore understood as meaning sequences which encode fusion proteins consisting of a signal peptide sequence.

Polyadenylation signals which are suitable as genetic control sequences are plant polyadenylation signals, preferably those which correspond essentially to T-DNA polyadenylation signals from Agrobacterium tumefaciens. Examples of particularly suitable terminator sequences are the OCS (octopine synthase) terminator and the NOS (nopaline synthase) terminator.

The DNA-constructs of the invention may encompass further nucleic acid sequences. Such nucleic acid sequences may preferably constitute expression cassettes. Said further sequences may include but shall not be limited to:
i) Additional negative, positive or counter selection marker as described above.
ii) Report genes which encode readily quantifiable proteins and which, via intrinsic color or enzyme activity, ensure the assessment of the transformation efficacy or of the location or timing of expression. Very especially preferred here are genes encoding reporter proteins (see also Schenborn E, Groskreutz D. Mol Biotechnol. 1999; 13(1):29-44) such as
   “green fluorescence protein” (GFP) (Chui W L et al., Curr Biol 1996, 6:325-330; Leffel S M et al., Biotechniques. 23(5):912-8, 1997; Sheen et al. (1995) Plant Journal 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228).
   Chloramphenicol transferase,
   luciferase (Millar et al., Plant Mol Biol Rep 1992 10:324-414; Ow et al. (1986) Science, 234:856-859); permits the detection of bioluminescence,
   β-galactosidase, encodes an enzyme for which a variety of chromogenic substrates are available,
   β-glucuronidase (GUS) (Jefferson et al., EMBO J. 1987, 6, 3901-3907) or the uidA gene, which encodes an enzyme for a variety of chromogenic substrates,
   R locus gene product: protein which regulates the production of anthocyanin pigments (red coloration) in plant tissue and thus makes possible the direct analysis of the promoter activity without the addition of additional adjuvants or chromogenic substrates (Dellaporta et al., In: Chromosome Structure and Function: Impact of New Concepts, 18th Stadler Genetics Symposium, 11:263-282, 1988),
   β-lactamase (Sutcliffe (1978) Proc Natl Acad Sci USA 75:3737-3741), enzyme for a variety of chromogenic substrates (for example PADAC, a chromogenic cephalosporin),
   xylE gene product (Zukowsky et al. (1983) Proc Natl Acad Sci USA 80:1101-1105), catechol dioxygenase capable of converting chromogenic catechols,
   alpha-amylase (Ikuta et al. (1990) Bio/technol. 8:241-242),
   tyrosinase (Katz et al. (1983) J Gene Microbiol 129:2703-2714), enzyme which oxidizes tyrosine to give DOPA and dopaquinone which subsequently form melanine, which is readily detectable,
   aequorin (Prasher et al. (1985) Biochem Biophys Res Commun 126(3):1259-1268), can be used in the calcium-sensitive bioluminescence detection.

The DNA construct according to the invention and any vectors derived therefrom may comprise further functional elements. The term “further functional elements” is to be understood in the broad sense. It preferably refers to all those elements which affect the generation, multiplication, function, use or value of said DNA construct or vectors comprising said DNA construct, or cells or organisms comprising the before mentioned. These further functional elements may include but shall not be limited to:
i) Origins of replication which ensure replication of the expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989).
ii) Multiple cloning sites (MCS) to enable and facilitate the insertion of one or more nucleic acid sequences.
iii) Sequences which make possible homologous recombination or insertion into the genome of a host organism.
iv) Elements, for example border sequences, which make possible the Agrobacterium-mediated transfer in plant cells for the transfer and integration into the plant genome, such as, for example, the right or left border of the T-DNA or the vir region.

### V. CONSTRUCTION OF THE DNA CONSTRUCTS OF THE INVENTION

Typically, constructs to be introduced into these cells are prepared using transgene expression techniques. Recombinant expression techniques involve the construction of recombinant nucleic acids and the expression of genes in transfected cells.

Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Vol. 152, Academic Press, hic., San Diego, Calif. (Berger); T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and Current Protocols in Molecular Biology, F. M. Ausubel et al., eds., Current Protocols, a joint venture between Greene Publishing Associates, Inc. and John Wiley & Sons, Inc., (1998 Supplement). Preferably, the DNA construct according to the invention is generated by joining the abovementioned essential constituents of the DNA construct together in the abovementioned sequence using the recombination and cloning techniques with which the skilled worker is familiar.

Generally, a gene to be expressed will be present in an expression cassette, meaning that the gene is operably linked to expression control signals, e.g., promoters and terminators, that are functional in the host cell of interest. The genes that encode the sequence-specific DNA cleaving enzyme and, optionally, the selectable marker, will also be under the control of such signals that are functional in the host cell. Control of expression is most easily achieved by selection of a promoter. The transcription terminator is not generally as critical and a variety of known elements may be used so long as they are recognized by the cell. The invention contemplates polynucleotides operably linked to a promoter in the sense or antisense orientation.

A DNA construct of the invention (or a expression cassette or other nucleic acid employed herein) is preferably introduced into cells using vectors into which these constructs or cassettes are inserted. Examples of vectors may be plasmids, cosmids, phages, viruses, retroviruses or else agrobacteria.

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress™ Expression System, Qiagen). The isolated and purified plasmids can then be further manipulated to produce other plasmids, used to transfect cells or incorporated into Agrobacterium tumefaciens to infect and transform plants. Where Agrobacterium is the means of transformation, shuttle vectors are constructed.

However, an expression cassette (e.g., for an excision enzyme) may also be constructed in such a way that the nucleic acid sequence to be expressed (for example one encoding an excision enzyme) is brought under the control of an endogenous genetic control element, for example a promoter, for example by means of homologous recombination or else by random insertion. Such constructs are likewise understood as being expression cassettes for the purposes of the invention. The skilled worker furthermore knows that nucleic acid molecules may also be expressed using artificial transcription factors of the zinc finger protein type (Beerli R R et al. (2000) Proc Natl Acad Sci USA 97(4):1495-500). These factors can be adapted to suit any sequence region and enable expression independently of certain promoter sequences.

### VI. TARGET ORGANISMS

The methods of the invention are useful for obtaining marker-free plants, or cells, parts, tissues, harvested material derived therefrom. Accordingly, another subject matter of the invention relates to transgenic plants or plant cells comprising in their genome, preferably in their nuclear, chromosomal DNA, the DNA construct according to the invention, and to cells, cell cultures, tissues, parts or propagation material—such as, for example, in the case of plant organisms leaves, roots, seeds, fruit, pollen and the like—derived from such plants.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom.

Preferred are plants and plant materials of the following plant families: Amaranthaceae, Brassicaceae, Carophyllaceae, Chenopodiaceae, Compositae, Cucurbitaceae, Labiatae, Leguminosae, Papilionoideae, Liliaceae, Linaceae, Malvaceae, Rosaceae, Saxifragaceae, Scrophulariaceae, Solanaceae, Tetragoniaceae.

Annual, perennial, monocotyledonous and dicotyledonous plants are preferred host organisms for the generation of transgenic plants. The use of the recombination system, or method according to the invention is furthermore advantageous in all ornamental plants, forestry, fruit, or ornamental trees, flowers, cut flowers, shrubs or turf. Said plant may include—but shall not be limited to—bryophytes such as, for example, Hepaticae (hepaticas) and Musci (mosses); pteridophytes such as ferns, horsetail and clubmosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae.

Plants for the purposes of the invention may comprise the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geraniums, Liliaceae such as drachaena, Moraceae such as ficus, Araceae such as philodendron and many others.

The transgenic plants according to the invention are furthermore selected in particular from among dicotyledonous crop plants such as, for example, from the families of the Leguminosae such as pea, alfalfa and soybean; the family of the Umbelliferae, particularly the genus Daucus (very particularly the species carota (carrot)) and Apium (very particularly the species graveolens dulce (celery)) and many others; the family of the Solanaceae, particularly the genus Lycopersicon, very particularly the species esculentum (tomato) and the genus Solanum, very particularly the species tuberosum (potato) and melongena (aubergine), tobacco and many others; and the genus Capsicum, very particularly the species annum (pepper) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli); and the genus Arabidopsis, very particularly the species thaliana and many others; the family of the Compositae, particularly the genus Lactuca, very particularly the species sativa (lettuce) and many others.

The transgenic plants according to the invention are selected in particular among monocotyledonous crop plants, such as, for example, cereals such as wheat, barley, sorghum and millet, rye, triticale, maize, rice or oats, and sugar cane.

Further preferred are trees such as apple, pear, quince, plum, cherry, peach, nectarine, apricot, papaya, mango, and other woody species including coniferous and deciduous trees such as poplar, pine, sequoia, cedar, oak, etc.

Especially preferred are Arabidopsis thaliana, Nicotiana tabacum, oilseed rape, soy-bean, corn (maize), wheat, linseed, potato and tagetes.

Plant varieties may be excluded, particularly registrable plant varieties according to Plant Breeders Rights. It is noted that a plant need not be considered a “plant variety” simply because it contains stably within its genome a transgene, introduced into a cell of the plant or an ancestor thereof.

In addition to a plant, the present invention provides any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated off-spring, clone or descendant of such a plant, or any part or propagule of said plant, off-spring, clone or descendant.

Plant organisms are furthermore, for the purposes of the invention, other organisms which are capable of photosynthetic activity, such as, for example, algae or cyanobacteria, and also mosses. Preferred algae are green algae, such as, for example, algae of the genus Haematococcus, Phaedactylum tricornatum, Volvox or Dunaliella.

Genetically modified plants according to the invention which can be consumed by humans or animals can also be used as food or feedstuffs, for example directly or following processing known in the art.

### VII. METHODS FOR INTRODUCING CONSTRUCTS INTO TARGET CELLS

A DNA construct according to the invention may advantageously be introduced into cells using vectors into which said DNA construct is inserted. Examples of vectors may be plasmids, cosmids, phages, viruses, retroviruses or agrobacteria. In an advantageous embodiment, the expression cassette is introduced by means of plasmid vectors. Preferred vectors are those which enable the stable integration of the expression cassette into the host genome.

The DNA construct can be introduced into the target plant cells and/or organisms by any of the several means known to those of skill in the art, a procedure which is termed transformation (see also Keown et al. (1990) Meth Enzymol 185:527-537). Production of stable, fertile transgenic plants in almost all economically relevant monocot plants is now routine: (Toriyama, et al. (1988) Bio/Technology 6:1072-1074; Zhang et al. (1988) Plant Cell Rep. 7:379-384; Zhang, et al. (1988) Theor Appl Genet. 76:835-840; Shimamoto et al. (1989) Nature 5338:274-276; Datta et al. (1990) Bio/Technology 8:736-740; Christou et al. (1991) Bio/Technology 9:957-962; Peng, et al. (1991) International Rice Research Institute, Manila, Philippines 563-574; Cao et al. (1992) Plant Cell Rep 11:585-591; Li et al. (1993) Plant Cell Rep. 12:250-255; Rathore et al. (1993) Plant Mol Biol 21:871-884; Fromm et al. (1990) Bio/Technology 8:833-839; Gordon-Kamm et al. (1990) Plant Cell 2:603-618; D'Halluin et al. (1992) Plant Cell 4:1495-1505; Walters et al. (1992) Plant Mol Biol 18:189-200; Koziel et al. (1993) Biotechnology 11:194-200; Vasil I K (1994) Plant Mol Biol 25, 925-937; Weeks et al. 11993) Plant Physiology 102, 1077-1084; Somers et al. (1992) Bio/Technology 10, 1589-1594; WO 92/14828).

For instance, the DNA constructs can be introduced into cells, either in culture or in the organs of a plant by a variety of conventional techniques. For example, the DNA constructs can be introduced directly to plant cells using ballistic methods, such as DNA particle bombardment, or the DNA construct can be introduced using techniques such as electroporation and microinjection of a cell. Particle-mediated transformation techniques (also known as “biolistics”) are described in, e.g., Klein et al. (1987) Nature 327:70-73; Vasil Vet al. (1993) Bio/Technol 11:1553-1558; and Becker D et al. (1994) Plant J 5:299-307. These methods involve penetration of cells by small particles with the nucleic acid either within the matrix of small beads or particles, or on the surface. The biolistic PDS-1000 Gene Gun (Biorad, Hercules, Calif.) uses helium pressure to accelerate DNA-coated gold or tungsten microcarriers toward target cells. The process is applicable to a wide range of tissues and cells from organisms, including plants. Other transformation methods are also known to those of skill in the art.

Microinjection techniques are known in the art and are well described in the scientific and patent literature. Also, the cell can be permeabilized chemically, for example using polyethylene glycol, so that the DNA can enter the cell by diffusion. The DNA can also be introduced by protoplast fusion with other DNA-containing units such as minicells, cells, lysosomes or liposomes. The introduction of DNA constructs using polyethylene glycol (PEG) precipitation is described in Paszkowski et al. (1984) EMBO J. 3:2717. Liposome-based gene delivery is e.g., described in WO 93/24640; Mannino and Gould-Fogerite (1988) BioTechniques 6(7):682-691; U.S. Pat. No. 5,279,833; WO 91/06309; and Felgner et al. (1987) Proc Natl Acad Sci USA 84:7413-7414).

Another suitable method of introducing DNA is electroporation, where the cells are permeabilized reversibly by an electrical pulse. Electroporation techniques are described in Fromm et al. (1985) Proc Natl Acad Sci USA 82:5824. PEG-mediated trans-formation and electroporation of plant protoplasts are also discussed in Lazzeri P (1995) Methods Mol Biol 49:95-106. Preferred general methods which may be mentioned are the calcium-phosphate-mediated transfection, the DEAE-dextran-mediated transfection, the cationic lipid-mediated transfection, electroporation, transduction and infection. Such methods are known to the skilled worker and described, for example, in Davis et al., Basic Methods In Molecular Biology (1986). For a review of gene transfer methods for plant and cell cultures, see, Fisk et al. (1993) Scientia Horticulturae 55:5-36 and Potrykus (1990) CIBA Found Symp 154:198.

Methods are known for introduction and expression of heterologous genes in both monocot and dicot plants. See, e.g., U.S. Pat. No. 5,633,446, U.S. Pat. No. 5,317,096, U.S. Pat. No. 5,689,052, U.S. Pat. No. 5,159,135, and U.S. Pat. No. 5,679,558; Weising et al. (1988) Ann. Rev. Genet. 22: 421-477. Transformation of monocots in particular can use various techniques including electroporation (e.g., Shimamoto et al. (1992) Nature 338:274-276; biolistics (e.g., EP-A1 270,356); and Agrobacterium (e.g., Bytebier et al. (1987) Proc Natl Acad Sci USA 84:5345-5349). In particular, Agrobacterium mediated transformation is now a highly efficient transformation method in monocots (Hiei et al. (1994) Plant J 6:271-282). Aspects of the invention provide an expression vector for use in such transformation methods which is a disarmed Agrobacterium Ti plasmid, and an Agrobacterium tumefaciens bacteria comprising such an expression vector. The generation of fertile transgenic plants has been achieved using this approach in the cereals rice, maize, wheat, oat, and barley (reviewed in Shimamoto K (1994) Current Opinion in Biotechnology 5:158-162; Vasil et al. (1992) Bio/Technology 10:667-674; Vain et al. (1995) Biotechnology Advances 13(4):653-671; Vasil (1996) Nature Biotechnology 14:702; Wan & Lemaux (1994) Plant Physiol. 104:37-48)

Other methods, such as microprojectile or particle bombardment (U.S. Pat. No. 5,100,792, EP-A-444 882, EP-A-434 616), electroporation (EP-A 290 395, WO 87/06614), microinjection (WO 92/09696, WO 94/00583, EP-A 331 083, EP-A 175 966, Green et al. (1987) Plant Tissue and Cell Culture, Academic Press) direct DNA uptake (DE 4005152, WO 90/12096, U.S. Pat. No. 4,684,611), liposome mediated DNA uptake (e.g. Freeman et al. (1984) Plant Cell Physiol 2 9:1353), or the vortexing method (e.g., Kindle (1990) Proc Natl Acad Sci USA 87:1228) may be preferred where Agrobacterium transformation is inefficient or ineffective.

In particular, transformation of gymnosperms, such as conifers, may be performed using particle bombardment 20 techniques (Clapham D et al. (2000) Scan J For Res 15: 151-160). Physical methods for the transformation of plant cells are reviewed in Oard, (1991) Biotech. Adv. 9 :1-11. Alternatively, a combination of different techniques may be employed to enhance the efficiency of the transformation process, e.g. bombardment with Agrobacterium coated microparticles (EP-A-486234) or microprojectile bombardment to induce wounding followed by co-cultivation with Agrobacterium (EP-A-486233).

In plants, methods for transforming and regenerating plants from plant tissues or plant cells with which the skilled worker is familiar are exploited for transient or stable transformation. Suitable methods are especially protoplast transformation by means of poly-ethylene-glycol-induced DNA uptake, biolistic methods such as the gene gun (“particle bombardment” method), electroporation, the incubation of dry embryos in DNA-containing solution, sonication and microinjection, and the transformation of intact cells or tissues by micro- or macroinjection into tissues or embryos, tissue electroporation, or vacuum infiltration of seeds. In the case of injection or electroporation of DNA into plant cells, the plasmid used does not need to meet any particular requirement. Simple plasmids such as those of the pUC series may be used. If intact plants are to be regenerated from the transformed cells, the presence of an additional selectable marker gene on the plasmid is useful.

In addition to these “direct” transformation techniques, transformation can also be carried out by bacterial infection by means of Agrobacterium tumefaciens or Agrobacterium rhizogenes. These strains contain a plasmid (Ti or Ri plasmid). Part of this plasmid, termed T-DNA (transferred DNA), is transferred to the plant following agrobacterial infection and integrated into the genome of the plant cell.

For Agrobacterium-mediated transformation of plants, the DNA construct of the invention may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Agrobacterium tumefaciens-mediated transformation techniques are well described in the scientific literature. See, for example, Horsch et al. (1984) Science 233:496-498, Fraley et al. (1983) Proc Natl Acad Sci USA 80:4803-4807, Hooykaas (1989) Plant Mol Biol 13:327-336, Horsch R B (1986) Proc Natl Acad Sci USA 83(8):2571-2575), Bevans et al. (1983) Nature 304:184-187, Bechtold et al. (1993) Comptes Rendus De L'Academie Des Sciences Serie III-Sciences De La Vie-Life Sciences 316:1194-1199, Valvekens et al. (1988) Proc Natl Acad Sci USA 85:5536-5540.

The DNA construct is preferably integrated into specific plasmids, either into a shuttle, or intermediate, vector or into a binary vector). If, for example, a Ti or Ri plasmid is to be used for the transformation, at least the right border, but in most cases the right and the left border, of the Ti or Ri plasmid T-DNA is linked with the expression cassette to be introduced as a flanking region. Binary vectors are preferably used. Binary vectors are capable of replication both in E. coli and in Agrobacterium. As a rule, they contain a selection marker gene and a linker or polylinker flanked by the right or left T-DNA flanking sequence. They can be transformed directly into Agrobacterium (Holsters et al. (1978) Mol Gen Genet 163:181-187). The selection marker gene permits the selection of transformed agrobacteria and is, for example, the DAAO gene of the invention, which imparts resistance to D-amino acids like D-alanine (see above). The Agrobacterium, which acts as host organism in this case, should already contain a plasmid with the vir region. The latter is required for transferring the T-DNA to the plant cell. An Agrobacterium thus transformed can be used for transforming plant cells.

Many strains of Agrobacterium tumefaciens are capable of transferring genetic material—for example the DNA construct according to the invention—, such as, for example, the strains. EHA101[pEHA101] (Hood E E et al. (1996) J Bacteriol 168(3):1291-1301), EHA105[pEHA105] (Hood et al. 1993, Transgenic Research 2, 208-218), LBA4404[pAL4404] (Hoekema et al. (1983) Nature 303:179-181), C58C1[pMP90] (Koncz and Schell (1986) Mol Gen Genet 204, 383-396) and C58C1[pGV2260] (Deblaere et al. (1985) Nucl Acids Res. 13, 4777-4788).

The agrobacterial strain employed for the transformation comprises, in addition to its disarmed Ti plasmid, a binary plasmid with the T-DNA to be transferred, which, as a rule, comprises a gene for the selection of the transformed cells and the gene to be transferred. Both genes must be equipped with transcriptional and translational initiation and termination signals. The binary plasmid can be transferred into the agrobacterial strain for example by electroporation or other transformation methods (Mozo & Hooykaas (1991) Plant Mol Biol 16:917-918). Co-culture of the plant explants with the agrobacterial strain is usually performed for two to three days.

A variety of vectors could, or can, be used. In principle, one differentiates between those vectors which can be employed for the agrobacterium-mediated transformation or agroinfection, i.e. which comprise the DNA construct of the invention within a T-DNA, which indeed permits stable integration of the T-DNA into the plant genome. Moreover, border-sequence-free vectors may be employed, which can be transformed into the plant cells for example by particle bombardment, where they can lead both to transient and to stable expression.

The use of T-DNA for the transformation of plant cells has been studied and described intensively (EP-A1 120 516; Hoekema, In: The Binary Plant Vector System, Offset-drukkerij Kanters B. V., Alblasserdam, Chapter V; Fraley et al. (1985) Crit Rev Plant Sci 4:1-45 and An et al. (1985) EMBO J 4:277-287). Various binary vectors are known, some of which are commercially available such as, for example, pBIN19 (Clontech Laboratories, Inc. USA).

To transfer the DNA to the plant cell, plant explants are cocultured with Agrobacterium tumefaciens or Agrobacterium rhizogenes. Starting from infected plant material (for example leaf, root or stalk sections, but also protoplasts or suspensions of plant cells), intact plants can be regenerated using a suitable medium which may contain, for example, antibiotics or biocides for selecting transformed cells. The plants obtained can then be screened in the presence of the DNA introduced, in this case the DNA construct according to the invention. As soon as the DNA has integrated into the host genome, the genotype in question is, as a rule, stable and the insertion in question is also found in the subsequent generations. Preferably the stably transformed plant is selected using the method of the invention (however other selection schemes employing other selection markers comprised in the DNA construct of the invention may be used). The plants obtained can be cultured and hybridized in the customary fashion. Two or more generations should be grown in order to ensure that the genomic integration is stable and hereditary.

The abovementioned methods are described, for example, in B. Jenes et al., Techniques for Gene Transfer, in: Transgenic Plants, Vol. 1, Engineering and Utilization, edited by S D Kung and R Wu, Academic Press (1993), 128-143 and in Potrykus (1991) Annu Rev Plant Physiol Plant Molec Biol 42:205-225). The construct to be expressed is preferably cloned into a vector which is suitable for the transformation of Agrobacterium tumefaciens, for example pBin19 (Bevan et al. (1984) Nucl Acids Res 12:8711).

The DNA construct of the invention can be used to confer desired traits on essentially any plant. One of skill will recognize that after DNA construct is stably incorporated in transgenic plants and confirmed to be operable, it can be introduced into other plants by sexual crossing. Any of a number of standard breeding techniques can be used, depending upon the species to be crossed.

### VIII. REGENERATION OF TRANSGENIC PLANTS

Transformed cells, i.e. those which comprise the DNA integrated into the DNA of the host cell, can be selected from untransformed cells preferably using the selection method of the invention. As soon as a transformed plant cell has been generated, an intact plant can be obtained using methods known to the skilled worker. For example, callus cultures are used as starting material. The formation of shoot and root can be induced in this as yet undifferentiated cell biomass in the known fashion. The shoots obtained can be planted and cultured.

Transformed plant cells, derived by any of the above transformation techniques, can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124176, Macmillian Publishing Company, New York (1983); and in Binding, Regeneration of Plants, Plant Protoplasts, pp. 21-73, CRC Press, Boca Raton (1985). Regeneration can also be obtained from plant callus, explants, somatic embryos (Dandekar et al. (1989) J Tissue Cult Meth 12:145; McGranahan et al. (1990) Plant Cell Rep 8:512), organs, or parts thereof. Such regeneration techniques are described generally in Klee et al. (1987) Ann Rev Plant Physiol 38:467-486. Other available regeneration techniques are reviewed in Vasil et al., Cell Culture and Somatic Cell Genetics of Plants, Vol I, II, and III, Laboratory Procedures and Their Applications, Academic Press, 1984, and Weissbach and Weissbach, Methods for Plant Molecular Biology, Academic Press, 1989.

### IX. GENERATION OF DESCENDANTS

After transformation, selection and regeneration of a transgenic plant (comprising the DNA construct of the invention) descendants are generated, which—because of the activity of the excision promoter—underwent excision and do not comprise the marker sequence(s) and expression cassette for the endonuclease.

Descendants can be generated by sexual or non-sexual propagation. Non-sexual propagation can be realized by introduction of somatic embryogenesis by techniques well known in the art. Preferably, descendants are generated by sexual propagation/fertilization. Fertilization can be realized either by selfing (self-pollination) or crossing with other transgenic or non-transgenic plants. The transgenic plant of the invention can herein function either as maternal or paternal plant.

After the fertilization process, seeds are harvested, germinated and grown into mature plants. Isolation and identification of descendants which underwent the excision process can be done at any stage of plant development. Methods for said identification are well known in the art and may comprise—for example—PCR analysis, Northern blot, Southern blot, or phenotypic screening (e.g., for an negative selection marker).

Descendants may comprise one or more copies of the agronomically valuable trait gene. Preferably, descendants are isolated which only comprise one copy of said trait gene. It is another inventive feature of the present invention that multiple insertion (e.g., of a T-DNA) in one genomic location will be reduced to a single insertion event by excision of the redundant copies (FIG. 10).

In a preferred embodiment the transgenic plant made by the process of the invention is marker-free. The terms “marker-free” or “selection marker free” as used herein with respect to a cell or an organisms are intended to mean a cell or an organism which is not able to express a functional selection marker protein (encoded by expression cassette b; as defined above) which was inserted into said cell or organism in combination with the gene encoding for the agronomically valuable trait. The sequence encoding said selection marker protein may be absent in part or—preferably—entirely. Furthermore the promoter operably linked thereto may be dysfunctional by being absent in part or entirely.

The resulting plant may however comprise other sequences which may function as a selection marker. For example the plant may comprise as a agronomically valuable trait a herbicide resistance conferring gene. However, it is most preferred that the resulting plant does not comprise any selection marker.

Also in accordance with the invention are cells, cell cultures, parts—such as, for example, in the case of transgenic plant organisms, roots, leaves and the like—derived from the above-described transgenic organisms, and transgenic propagation material (such as seeds or fruits).

Genetically modified plants according to the invention which can be consumed by humans or animals can also be used as food or feedstuffs, for example directly or following processing known per se. Here, the deletion of, for example, resistances to antibiotics and/or herbicides, as are frequently introduced when generating the transgenic plants, makes sense for reasons of customer acceptance, but also product safety.

A further subject matter of the invention relates to the use of the above-described transgenic organisms according to the invention and the cells, cell cultures, parts—such as, for example, in the case of transgenic plant organisms, roots, leaves and the like—derived from them, and transgenic propagation material such as seeds or fruits, for the production of food or feedstuffs, pharmaceuticals or fine chemicals. Here again, the deletion of, for example, resistances to antibiotics and/or herbicides is advantageous for reasons of customer acceptance, but also product safety.

Fine chemicals is understood as meaning enzymes, vitamins, amino acids, sugars, fatty acids, natural and synthetic flavors, aromas and colorants. Especially preferred is the production of tocopherols and tocotrienols, and of carotenoids. Culturing the trans-formed host organisms, and isolation from the host organisms or from the culture medium, is performed by methods known to the skilled worker. The production of pharmaceuticals such as, for example, antibodies or vaccines, is described by Hood E E, Jilka J M. (1999) Curr Opin Biotechnol. 10(4):382-386; Ma J K and Vine N D (1999) Curr Top Microbiol Immunol. 236:275-92).

Various further aspects and embodiments of the present invention will be apparent to those skilled in the art in view of the present disclosure. All documents mentioned in this specification are incorporated herein in their entirety by reference. Certain aspects and embodiments of the invention will now be illustrated by way of example and with reference to the figure described below.

### X. SEQUENCES

1. SEQ ID NO:1: Nucleic acid sequence encoding D-amino acid oxidase from Rhodosporidium toruloides (Yeast)
2. SEQ ID NO:2: Amino acid sequence encoding D-amino acid oxidase from Rhodosporidium toruloides (Yeast)
3. SEQ ID NO:3: Nucleic acid sequence encoding D-amino acid oxidase from Caenorhabditis elegans
4. SEQ ID NO:4: Amino acid sequence encoding D-amino acid oxidase from Caenorhabditis elegans
5. SEQ ID NO:5: Nucleic acid sequence encoding D-amino acid oxidase from Nectria haematococca
6. SEQ ID NO:6: Amino acid sequence encoding D-amino acid oxidase from Nectria haematococca
7. SEQ ID NO:7: Nucleic acid sequence encoding D-amino acid oxidase from Trigonopsis variabilis
8. SEQ ID NO:8: Amino acid sequence encoding D-amino acid oxidase from Trigonopsis variabilis
9. SEQ ID NO:9: Nucleic acid sequence encoding D-amino acid oxidase from Schizosaccharomyces pombe (fission yeast)
10. SEQ ID NO:10: Amino acid sequence encoding D-amino acid oxidase from Schizosaccharomyces pombe (fission yeast)
11. SEQ ID NO:11: Nucleic acid sequence encoding D-amino acid oxidase from Streptomyces coelicolor A3(2)
12. SEQ ID NO:12: Amino acid sequence encoding D-amino acid oxidase from Streptomyces coelicolor A3(2)
13. SEQ ID NO:13: Nucleic acid sequence encoding D-amino acid oxidase from Candida boidinii
14. SEQ ID NO:14: Amino acid sequence encoding D-amino acid oxidase from Candida boidinii
15. SEQ ID NO:15: Nucleic acid sequence encoding vector daaoSceITetON (length: 12466 bp)

| Feature | Position (Base No.) | Orientation |
|---|---|---|
| LB (Left Border) | 7618-7834 | direct |
| 35SpA (35S terminator) | 7345-7549 | complement |
| ptxA promoter | 6479-7341 | complement |
| rtTA (reverse tetracycline | 5418-6425 | complement |
| transcription transactivator) | | |
| OCS-T (OCS terminator) | 5118-5343 | complement |
| nit1P (Nit1 promoter) | 3217-5028 | complement |
| daao (D-amino acid oxidase) | 2067-3173 | complement |
| nosT (nos terminator) | 1735-1990 | complement |
| pTOP10P (tet regulated promoter) | 1270-1660 | complement |
| IScel (I-Scel endonuclease) | 515-1222 | complement |
| I-Sce recognition/cleavage site | 445-462 | direct |
| 35SpA (35S terminator) | 196-400 | complement |
| RB (right border) | 38-183 | direct |

16. SEQ ID NO:16: Nucleic acid sequence encoding vector daaoNit-PRecombination (length: 12539 bp)

| Feature | Position (Base No.) | Orientation |
|---|---|---|
| LB (Left Border) | 7691-7907 | direct |
| STPT (sTPT promoter) | 7619-6302 | complement |
| GUS (GUS gene) | 6248-4251 | complement |
| 35SpA (35S terminator) | 4176-3972 | complement |
| Nit1P (Nit1 promoter) | 3882-2071 | complement |
| daao (D-amino acid oxidase) | 2027-921 | complement |
| nosT (nos terminator) | 844-589 | complement |
| I-Sce recognition/cleavage site | 445-462 | direct |
| 35SpA (35S terminator) | 400-196 | complement |
| RB (right border) | 38-183 | direct |

### XI. BRIEF DESCRIPTION OF THE FIGURES

FIG. 1: Basic Principle of the dual-function selection marker
A: A mixture population consisting of wild-type, non-transgenic plants (gray color) and transgenic plants comprising the dual-function marker (black color) is treated with either D-alanine or D-isoleucine. While the toxic effect of D-alanine on non-transgenic plants is detoxified by the transgene-mediated conversion (thereby selectively killing the wild-type plantlets), the non-toxic D-isoleucine is converted by the same enzymatic mechanism into a phytotoxic compound (thereby selectively killing the transgenic plantlets).
B: The dual-function of the marker can be employed subsequently for construction of marker-free transgenic plants. While the function as a negative selection marker is utilized to allow for insertion of a transgene comprising a gene of interest (GOI) into a wild-type plant (gray color), the counter-selection-function is employed to subsequently delete the selection marker by combining marker-deletion technology and counter-selection (thereby killing the dual-function marker comprising plantlets (black-color)) resulting in plantlets comprising the GOI but lacking the dual function marker (gray hatching).

FIG. 2: Wild-type Arabidopsis thaliana plantlets (left side) and transgenic plantlets comprising the dual function marker (DAAO gene from Rhodotorula gracilis) are treated with either 30 mM D-isoleucine (upper side) or 30 mM D-alanine (bottom side). A toxic effect of D-isoleucine on the transgenic plants and D-alanine on the wild-type plants, respectively, can be observed, while no severe damage can be detected on the respective other group, thereby allowing for clear distinguishing and easy selection of either transgenic or wild-type plants.

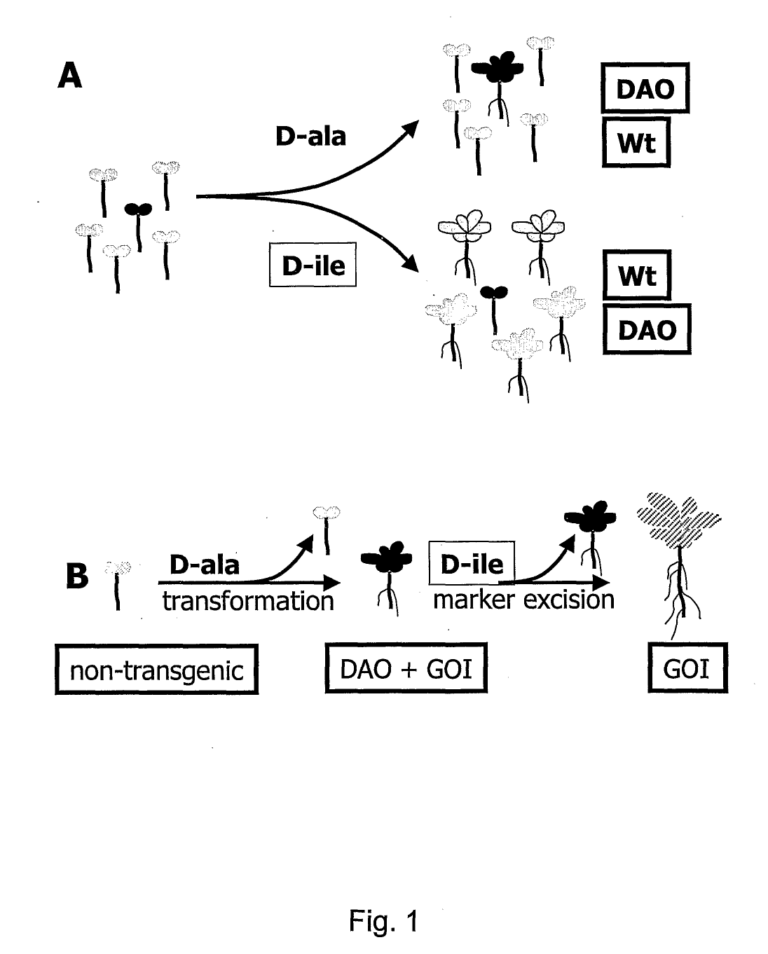

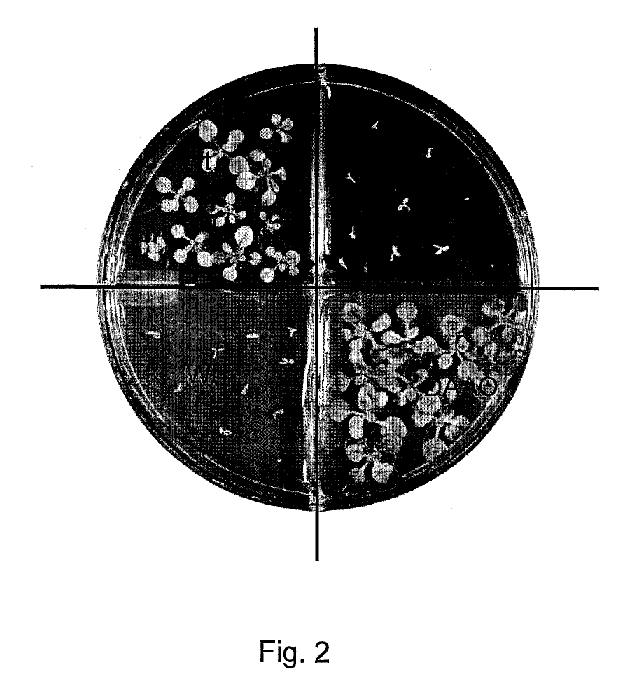

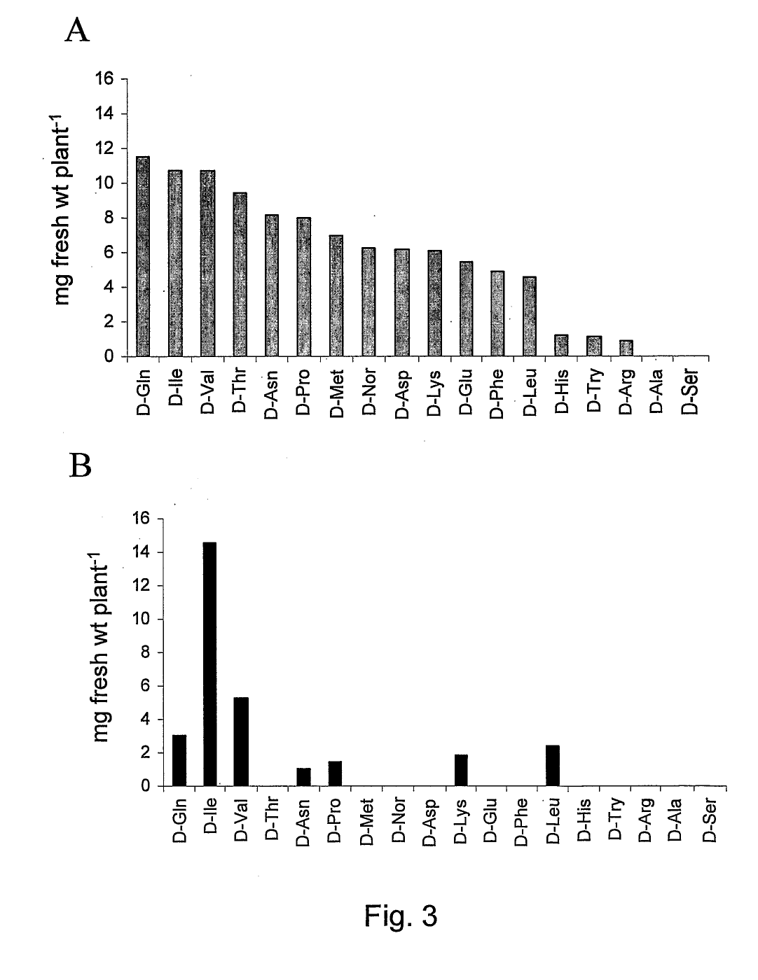

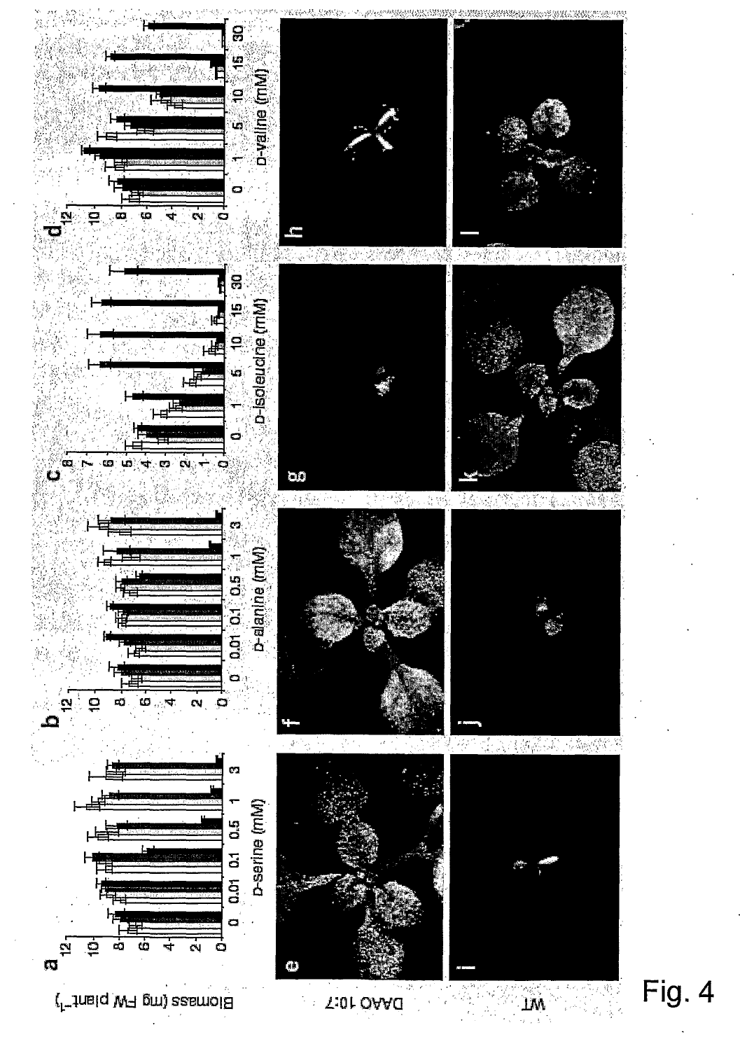

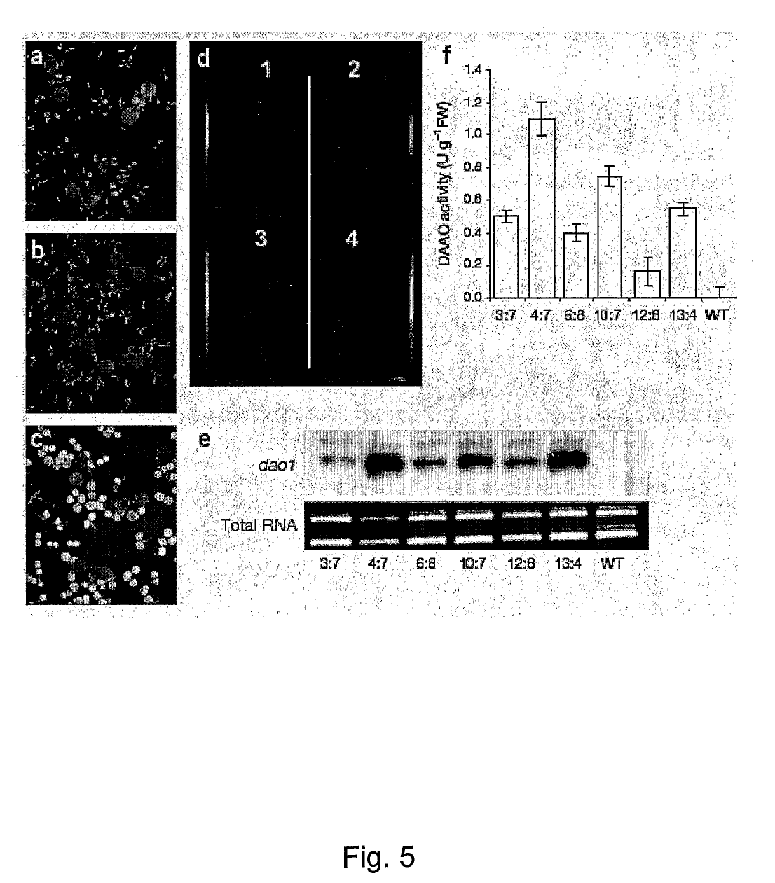

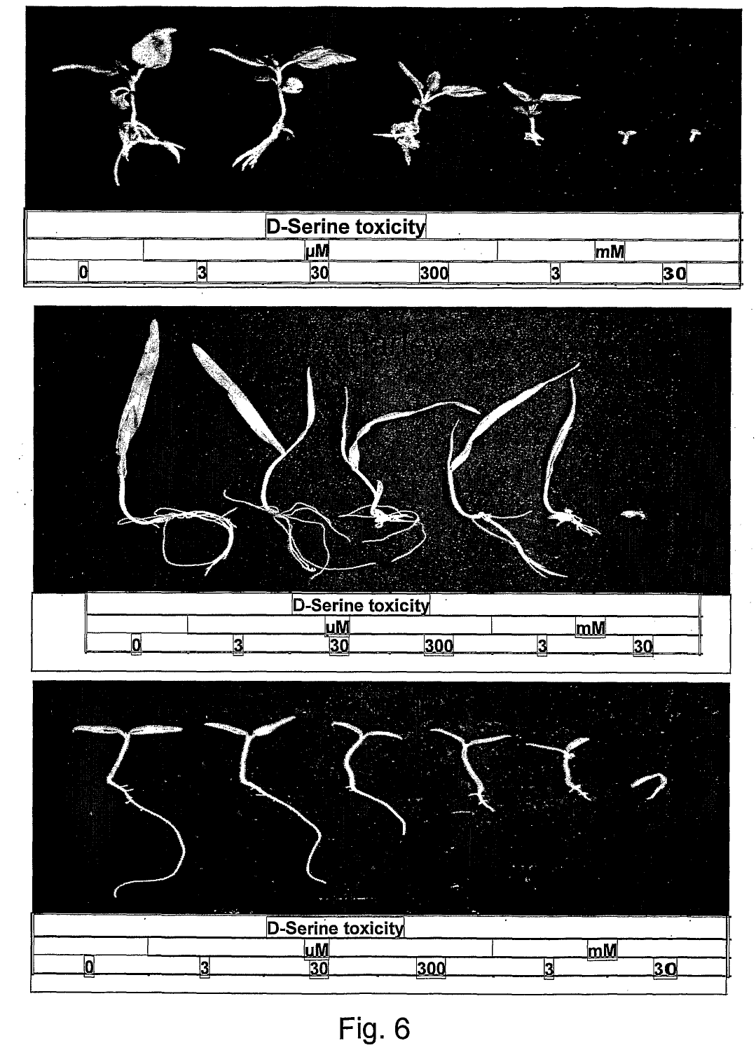

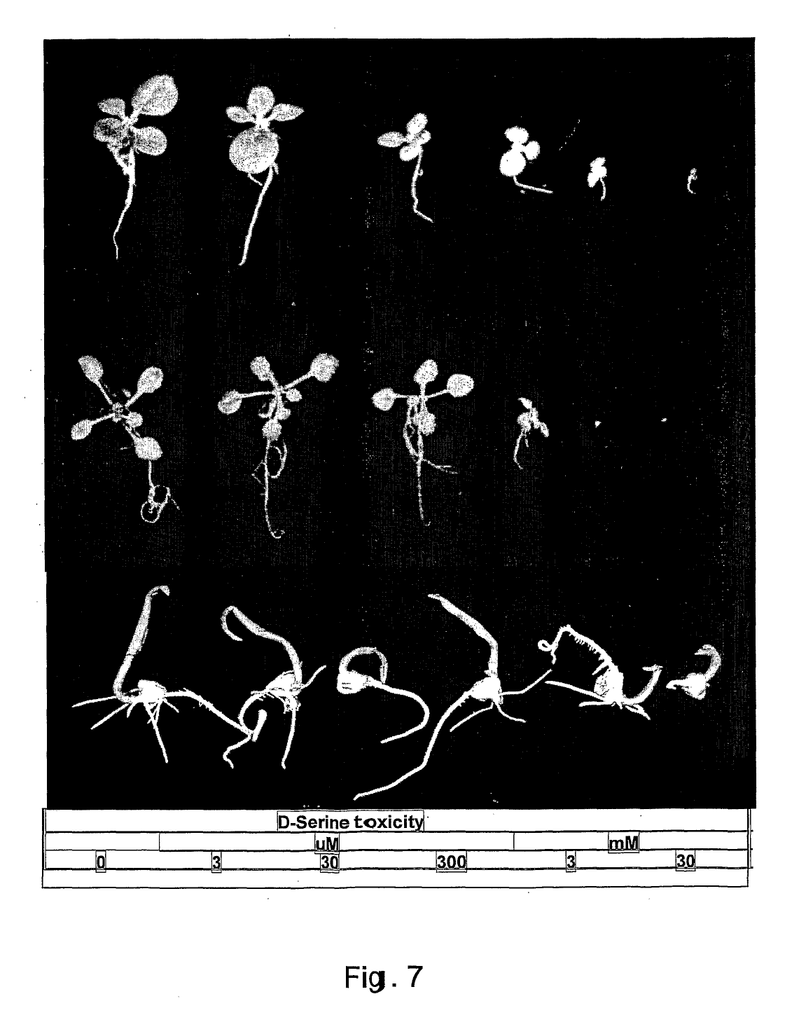

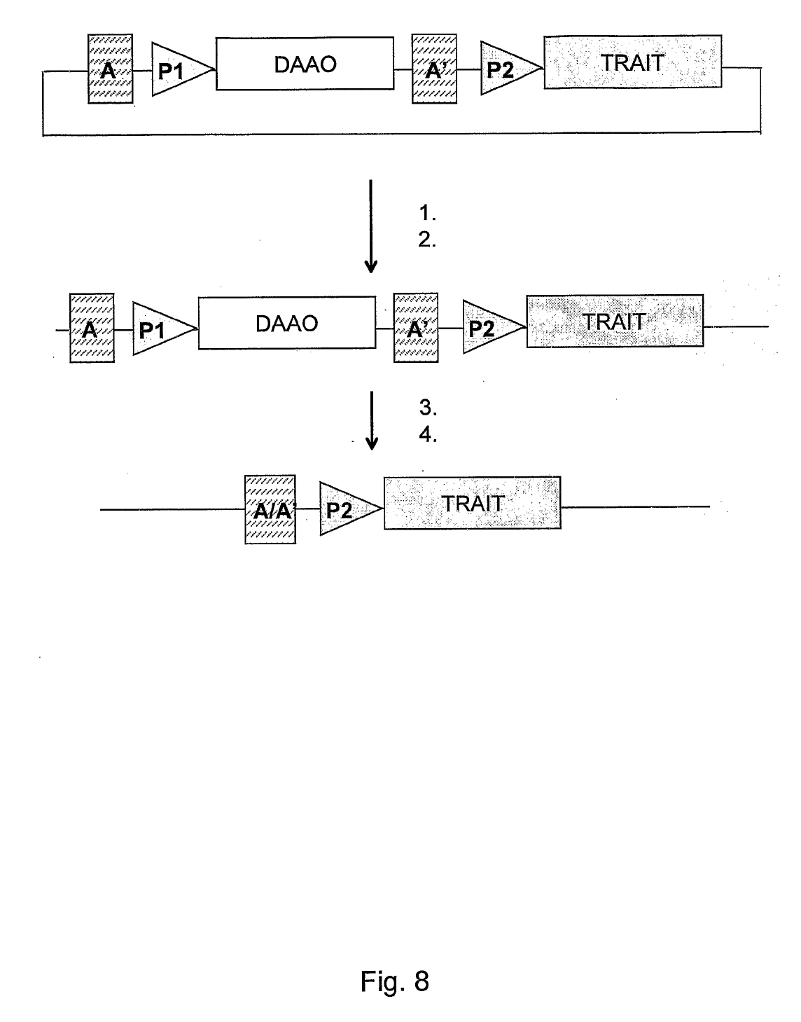

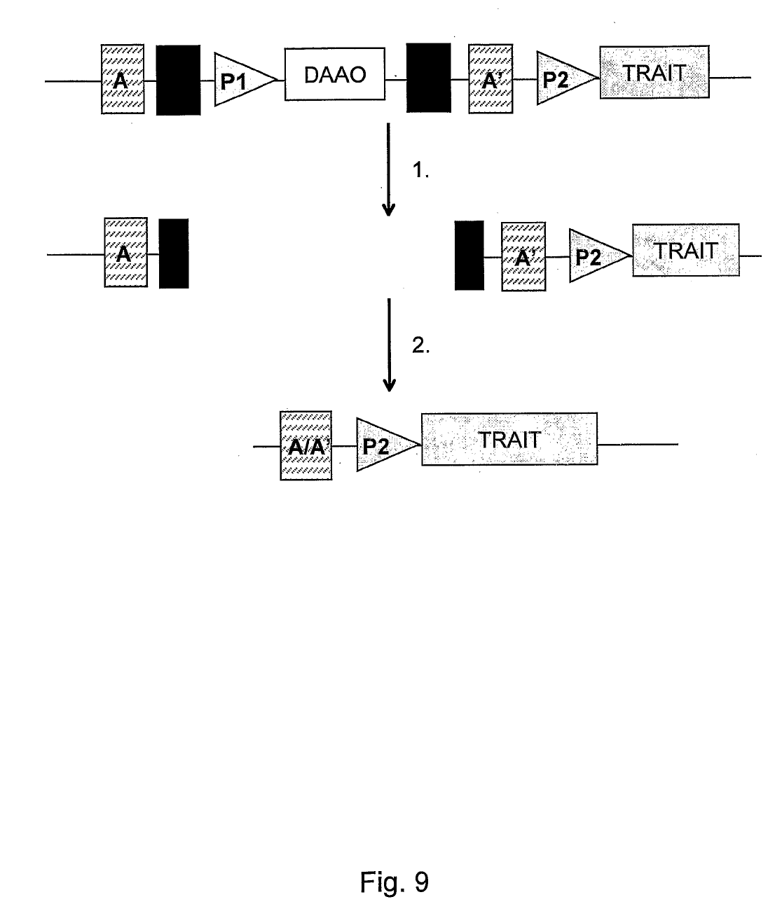

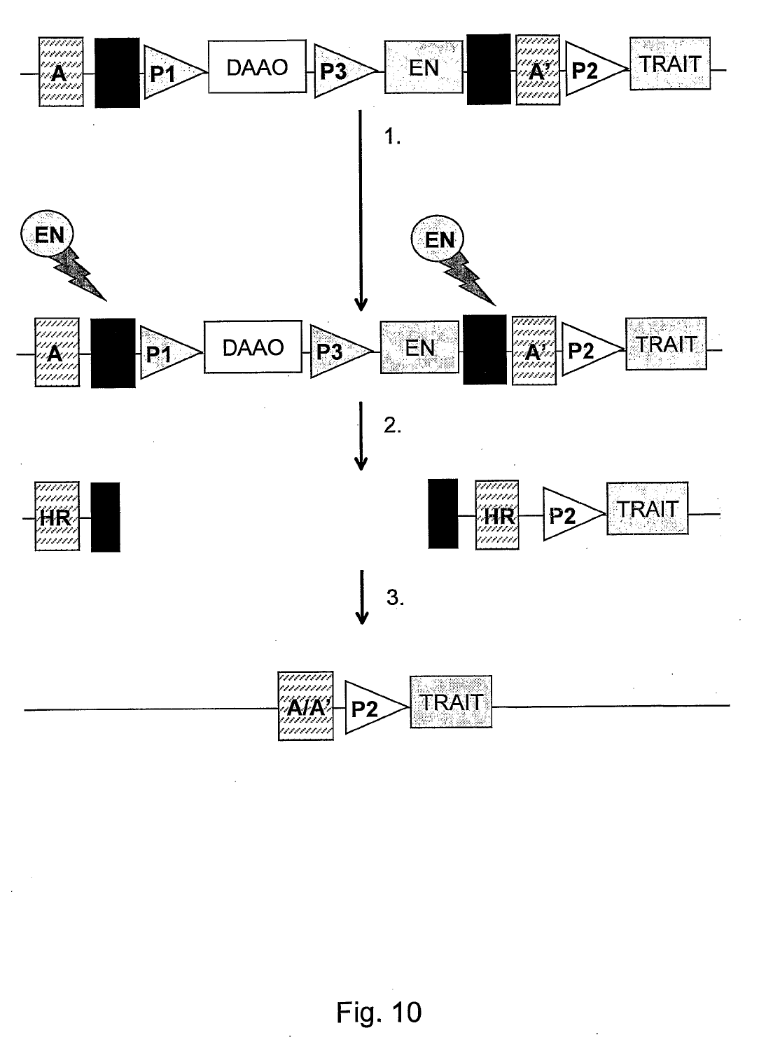

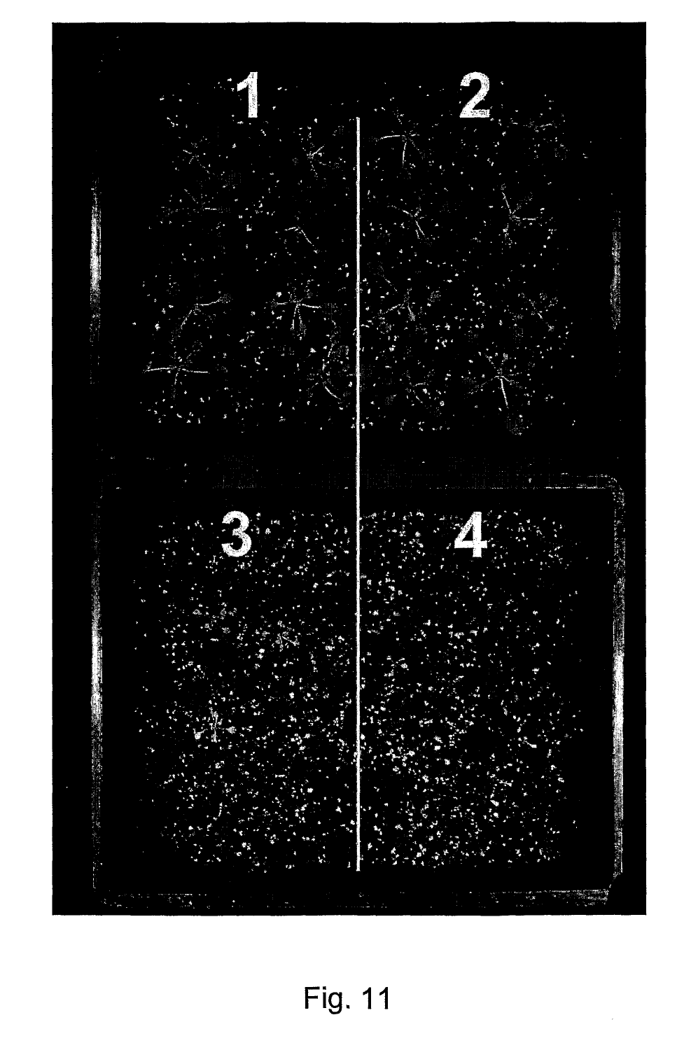

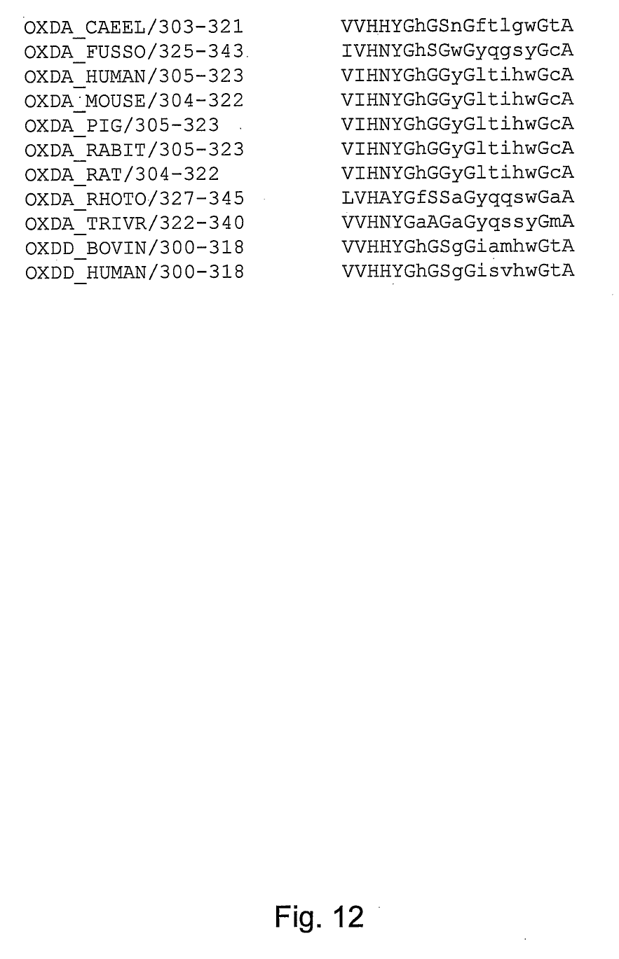

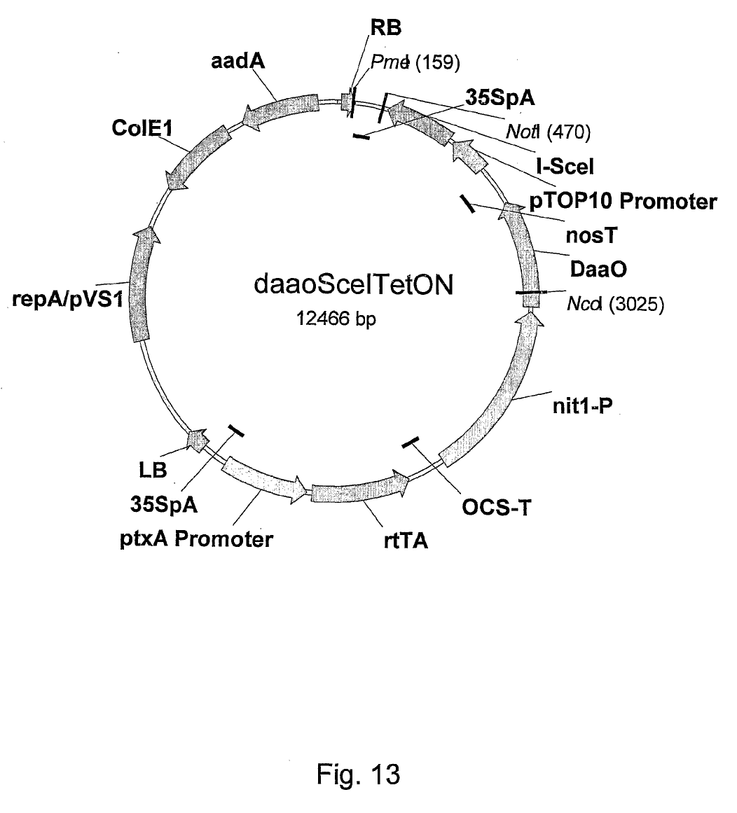

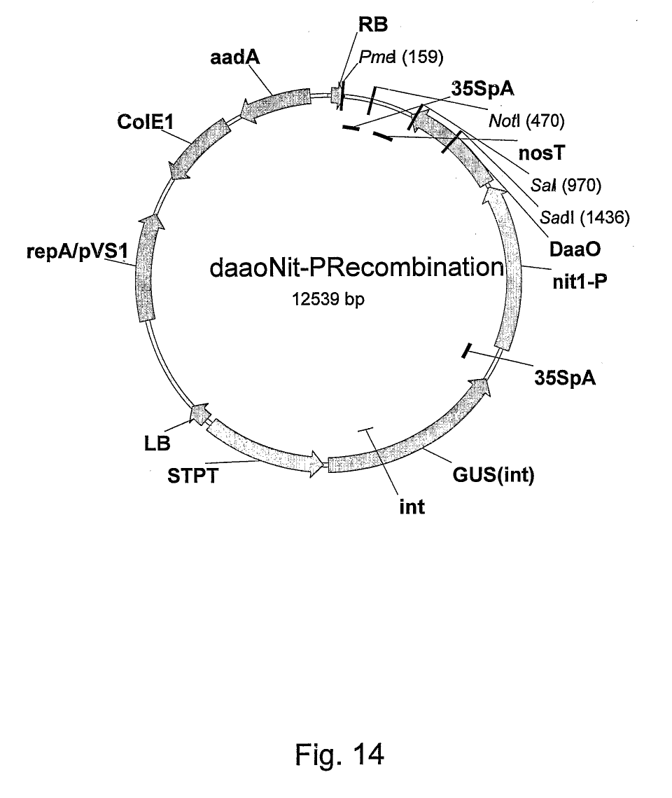

FIG. 3 Effect of various D-amino acids on plant growth.
Wild type Arabidopsis thaliana plantlets were grown on half-concentrated Murashige-Skoog medium (0.5% (wt/vol) sucrose, 0.8% (wt/vol) agar) supplemented with the indicated D-amino acid at either 3 mM (Panel A) or 30 mM (Panel B). While D-alanine and D-serine are imposing severe phytotoxic effects even at 3 mM concentrations no significant effects can be observed for D-isoleucine.

FIG. 4 D-amino acid dose responses of dao1 transgenic and wild-type A. thaliana.
(a-d) Growth of dao1 transgenic line 3:7 (white), 10:7 (light gray), 13:4 (gray) and wild-type (black) plants, in fresh weight per plant, on media containing various concentrations of D-serine, D-alanine, D-isoleucine and D-valine in half-strength MS with 0.5% (wt/vol) sucrose and 0.8% (wt/vol) agar. Different concentration ranges were used for different D-amino acids. The plants were grown for 10 d after germination under 16 h photoperiods at 24° C.; n=10±s.e.m., except for plants grown on D-isoleucine, where smaller Petri dishes were used, (n=6±s.e.m.).
(e-l) Photographs of dao1 transgenic line 10:7 (e-h) and wild-type plants (i-l), grown for 10 d on the highest concentrations of the D-amino acid shown in the respective graphs above. All pictures have the same magnification. FW, fresh weight.

FIG. 5: Selection of primary transformants with the DAAO marker.
(a-c) DAAO T1 seedlings on media containing 3 mM D-alanine (a), 3 mM D-serine (b) and 50 μg ml-1 kanamycin (c). Seeds were surface-sterilized and sown on half-strength MS plates with 0.5% (wt/vol) sucrose, 0.8% (wt/vol) agar and the respective selective compound, then grown for 5 d after germination under 16 h photoperiods at 24° C.
(d) DAAO transgenic plants grown on soil photographed after selection by spraying with (1) D-alanine and (2) D-serine, and wild-type plants sprayed with (3) D-alanine and (4) D-serine. Eight seeds per plot and treatment were sown on soil, and grown for 7 d after germination before applying the selective treatment, which consisted of spraying with aqueous 50 mM solutions of D-alanine or D-serine with 0.05% Tween 80 on three consecutive days.

(e) Northern blot analysis of dao1 mRNA levels from six D-serine- and D-alanine-resistant lines and wild-type plants. Ten μg total RNA was loaded per lane and separated on an agarose gel. Ethidium bromide-stained total RNA bands are shown as loading controls. (f) DAAO activity in six transgenic lines and wild type. A unit of DAAO activity is defined as the turnover of one micromole of substrate per minute. Bars represent means ±s.e.m., n=3

FIG. 6+7 Demonstration of broad applicability of the selection system. D-serine is imposing toxic effects on a variety of different plant species both monocotyledonous and dicotyledonous plants. Effects are demonstrated for popular (FIG. 6A), barley (FIG. 6B), tomato (FIG. 6C), tobacco (FIG. 7A), Arabidopsis thaliana (FIG. 7B), and Corn (Zea mays, FIG. 7C). Similar effects are obtained when using D-alanine instead of D-serine.

FIG. 8-10: Preferred constructs of the invention
The following abbreviations apply to the figures in general:
   A: Sequence A allowing for sequence deletion (e.g., recognition site for recombinase or homology sequence)
   A′: Sequence A′allowing for sequence deletion (e.g., recognition site for recombinase or homology sequence)
   A/A′: Sequence as the result of (homologous) recombination of A and A′
   DAAO: Sequence encoding a d-amino acid oxidase having dual-function marker activity.
   EN: Sequence encoding sequence specific DNA-endonuclease
   Trait: Sequence coding for e.g., agronomically valuable trait
   Pₙ: Promoter
   RSₙ: Recognition sequence for the site-directed induction of DNA double-strand breaks (e.g., S1: First recognition sequence). The recognition sequences may be different (e.g., functioning for different endonucleases) or—preferably—identical (but only placed in different locations).
   Rₙ or Sₙ: Part of recognition sequence RSₙ remaining after cleavage

FIG.: 8 Preferred basic construct and method
A vector comprising the DNA construct (preferably a circular Agrobacterium binary vector) is employed comprising: A first expression cassette for the dual-function marker (DAAO) under control of a promoter functional in plants (P1) and a second expression cassette for an agronomically valuable trait (TRAIT) also under control of (preferably a different) promoter functional in plants (P2). The first expression cassette is flanked by sequences which allow for specific deletion of said first expression cassette (A and A′). A and A′ may be sequences for a sequence-specific recombinase or sequences which allow for homologous recombination between each other. For the later alternative, two identical sequences can be arranged in form of a directed repeat.
The DNA construct is inserted into plant cells (1.) and selection is performed making use of the negative selection function of the dual function marker (2.) e.g., employing D-alanine or D-serine. Thereby plant cells or plants are selected comprising the DNA construct. Based on said plant cells or plants deletion of the first expression cassette is initiated (3.) and selection is performed making use of the counter-selection function of the dual function marker (4.) e.g., employing D-isoleucine or D-valine. Thereby plant cells or plants are selected comprising the second expression cassette but lacking the first expression cassette.

FIG.: 9 Construct mediating marker excision via induced homologous recombination The DNA construct introduced into the plant genome by utilizing the negative selection marker function of the dual-function marker is comprising: A first expression cassette for the dual-function marker (DAAO) under control of a promoter functional in plants (P1) and a second expression cassette for an agronomically valuable trait (TRAIT) also under control of (preferably a different) promoter functional in plants (P2). The first expression cassette is flanked by homology sequences A and A′ which allow for homologous recombination between each other, being arranged in form of a directed repeat. Within the DNA construct there is at least one (preferably—as depicted here—two) recognition sequences (RS) (cleavage sites) for a sequence specific endonuclease (RS₁, RS₂). The two sequences may be different (i.e., for different endonucleases) or—preferably—identical. Cleavage at said recognition sequences (RS₁ and RS₂) is initiated by the corresponding endonuclease (1.) resulting in double-strand breaks, which are “repaired” by homologous recombination between the homologous end-sequences (probably supported by the cellular DNA repair mechanism). The resulting genome still comprises the second expression cassette for the trait but lacks the first expression cassette for the dual-function marker. Selection is performed making use of the counter-selection function of the dual function marker (2.) e.g., employing D-isoleucine or D-valine. Thereby plant cells or plants are selected comprising the second expression cassette but lacking the first expression cassette.
In an preferred embodiment the DNA construct introduced into the plant genome further comprises a third expression cassette for the sequence specific endonuclease (or if recombinases are utilized for the recombinase). The first expression cassette (for the dual-function marker) and the third expression cassette (for the endonuclease) are together flanked by homology sequences A and A′ which allow for homologous recombination between each other, being arranged in form of a directed repeat. Within the DNA construct there is at least one (preferably—as depicted here—two) recognition sequences (RS) (cleavage sites) for a sequence specific endonuclease (RS₁, RS₂). The two sequences may be different (i.e., for different endonucleases) or—preferably—identical. Expression of the corresponding endonuclease from the third expression cassette is initiated (1.), resulting in cleavage at said recognition sequences (RS₁ and RS₂) thereby forming in double-strand breaks (2.), which are “repaired” by homologous recombination between the homologous end-sequences (probably supported by the cellular DNA repair mechanism). The resulting genome still comprises the second expression cassette for the trait but lacks the first and third expression cassette. Selection is performed making use of the counter-selection function of the dual function marker (3.) e.g., employing D-isoleucine or D-valine. Thereby plant cells or plants are selected comprising the second expression cassette but lacking the first and third expression cassette.
Preferably the expression of the endonuclease is controllable e.g., by employing an inducible promoter (see below for details).

FIG.: 11 D-amino acids are applicable by spraying procedure DAAO transgenic plants grown on soil photographed after selection by spraying with (1) D-alanine and (2) D-serine, and wild-type plants sprayed with (3) D-alanine and (4) D-serine. Eight seeds per plot and treatment were sown on soil, and grown for 7 d after germination before applying the selective treatment, which consisted of spraying with aqueous 50 mM solutions of D-alanine or D-serine with 0.05% Tween 80 on three consecutive days.

FIG.: 12 Alignment of the catalytic site of various D-amino acid oxidases
Multiple alignment of the catalytic site of various D-amino acid oxidases allows for determination of a characteristic sequence motif [LIVM]-[LIVM]-H*-[NHA]-Y-G-x-[GSA]-[GSA]-x-G-x₅-G-x-A, which allows for easy identification of additional D-amino acid oxidases suitable to be employed within the method and DNA-constructs of the invention.

FIG.: 13 Vector map of construct daaoSceITetOn (Seq ID NO: 15) (length: 12466 bp)

| | | Position | |
|---|---|---|---|
| Abbreviation | Feature | (Base No.) | Orientation |
| LB | Left Border | 7618-7834 | direct |
| 35SpA | 35S terminator | 7345-7549 | complement |
| ptxA promoter | | 6479-7341 | complement |
| rtTA | Tet repressor | 5418-6425 | complement |
| OCS-T | OCS terminator | 5118-5343 | complement |
| nit1P | Nit1 promoter | 3217-5028 | complement |
| daao | D-amino acid oxidase | 2067-3173 | complement |
| nosT | nos terminator | 1735-1990 | complement |
| pTOP10P | tet regulated promoter | 1270-1660 | complement |
| ISecl | I-Secl endonuclease | 515-1222 | complement |
| I-Sce recognition/cleavage site | | 445-462 | direct |
| 35SpA | 35S terminator | 196-400 | complement |
| RB | right border | 38-183 | direct |
| ColE1 | ColE1 origin of | | |
| | replication (E. coli) | | |
| aadA | Spectomycin/Strepotomycin | | |
| | resistance | | |
| repA/pVS1 | repA origin of replication | | |
| | (Agrobacterium) | | |

Furthermore, important restriction sites are indicated with their respective cutting position.

FIG.: 14 Vector map of construct daaoNit-PRecombination (Seq ID NO: 16) (length: 12539 bp)

| Abbreviation | Feature | Position (Base No.) | Orientation |
|---|---|---|---|
| LB | Left Border | 7691-7907 | direct |
| STPT | sTPT promoter | 7619-6302 | complement |
| GUS | GUS gene | 6248-4251 | complement |
| 35SpA | 35S terminator | 4176-3972 | complement |
| Nit1P | Nit1 promoter | 3882-2071 | complement |
| daao | D-amino acid oxidase | 2027-921 | complement |
| nosT | nos terminator | 844-589 | complement |
| I-Sce recognition/cleavage site | | 445-462 | direct |
| 35SpA | 35S terminator | 400-196 | complement |
| RB | right border | 38-183 | direct |
| ColE1 | ColE1 origin of | | |
| | replication (E. coli) | | |
| aadA | Spectomycin/ | | |
| | Strepotomycin | | |
| | resistance | | |
| repA/pVS1 | repA origin of | | |
| | replication | | |
| | (Agrobacterium) | | |

Furthermore, important restriction sites are indicated with their respective cutting position. The GUS gene is comprising an intron (int).

### XII. EXAMPLES

### General Methods

The chemical synthesis of oligonucleotides can be effected for example in the known manner using the phosphoamidite method (Voet, Voet, 2nd edition, Wiley Press New York, pages 896-897). The cloning steps carried out for the purposes of the pre-sent invention, such as, for example, restriction cleavages, agarose gel electrophoresis, purification of DNA fragments, the transfer of nucleic acids to nitrocellulose and nylon membranes, the linkage of DNA fragments, the transformation of E. coli cells, bacterial cultures, the propagation of phages and the sequence analysis of recombinant DNA are carried out as described by Sambrook et al. (1989) Cold Spring Harbor Laboratory Press; ISBN 0-87969-309-6. Recombinant DNA molecules were sequenced using an ALF Express laser fluorescence DNA sequencer (Pharmacia, Sweden) following the method of Sanger (Sanger et al., Proc. Natl. Acad. Sci. USA 74 (1977), 5463-5467).

### Example 1

### Vector Construction and Plant Transformation

DNA and RNA manipulation were done using standard techniques.

The yeast R. gracilis was grown in liquid culture containing 30 mM D-alanine to induce dao1, the gene encoding DAAO. Total RNA was isolated from the yeast and used for cDNA synthesis. The PCR primers

5′-ATTAGATCTTACTACTCGAAGGACGCCATG-3′
and
5′-ATTAGATCTACAGCCACAATTCCCGCCCTA-3′

were used to amplify the dao1 gene from the cDNA template by PCR. The PCR fragments were sub-cloned into the pGEM-T Easy vector (Promega) and subsequently ligated into the BamHI site of the CaMV 35S expression cassette of the binary vector pPCV702kana17 giving pPCV702:dao1. The vectors were subjected to restriction analysis and sequencing to check that they contained the correct constructs.

### Example 1a

### Transformation of Arabidopsis thaliana

A. thaliana plants (ecotype Col-0) were grown in soil until they flowered. Agrobacterium tumefaciens (strain GV3101:pMP110 RK) transformed with the construct of interest was grown in 500 mL in liquid YEB medium (5 g/L Beef extract, 1 g/L Yeast Extract (Duchefa), 5 g/L Peptone (Duchefa), 5 g/L sucrose (Duchefa), 0.49 g/L MgSO₄ (Merck)) until the culture reached an OD₆₀₀ 0.8-1.0. The bacterial cells were harvested by centrifugation (15 minutes, 5,000 rpm) and resuspended in 500 mL infiltration solution (5% sucrose, 0.05% SILWET L-77 [distributed by Lehle seeds, Cat. No. VIS-02]).

Flowering A. thaliana plants were then transformed by the floral dip method (Clough S J & Bent A F (1998) Plant J. 16, 735-743 (1998) with the transgenic Agrobacterium tumefaciens strain carrying the vector described above by dipping for 10-20 seconds into the Agrobacterium solution. Afterwards the plants were kept in the greenhouse until seeds could be harvested. Transgenic seeds were selected by plating surface sterilized seeds on growth medium A (4.4 g/L MS salts [Sigma-Aldrich], 0.5 g/L MES [Duchefa]; 8 g/L Plant Agar [Duchefa]) supplemented with 50 mg/L kanamycin for plants carrying the nptII resistance marker, or 0.3 to 30 mM D-amino acids (as described below) for plants comprising the dual-function marker of the invention. Surviving plants were transferred to soil and grown in the greenhouse.

Lines containing a single T-DNA insertion locus were selected by statistical analysis of T-DNA segregation in the T2 population that germinated on kanamycin or D-amino acid-containing medium. Plants with a single locus of inserted T-DNA were grown and self-fertilized. Homozygous T3 seed stocks were then identified by analyzing T-DNA segregation in T3 progenies and confirmed to be expressing the introduced gene by northern blot analyses.

### Example 1b

### Agrobacterium-Mediated Transformation of Brassica napus

Agrobacterium tumefaciens strain GV3101 transformed with the plasmid of interest was grown in 50 mL YEB medium (see Example 4a) at 28° C. overnight. The Agrobacterium solution is mixed with liquid co-cultivation medium (double concentrated MSB5 salts (Duchefa), 30 g/L sucrose (Duchefa), 3.75 mg/l BAP (6-benzylamino purine, Duchefa), 0.5 g/l MES (Duchefa), 0.5 mg/l GA3 (Gibberellic Acid, Duchefa); pH5.2) until OD₆₀₀ of 0.5 is reached. Petiols of 4 days old seedlings of Brassica napus cv. Westar grown on growth medium B (MSB5 salts (Duchefa), 3% sucrose (Duchefa), 0.8% oxoidagar (Oxoid GmbH); pH 5, 8) are cut. Petiols are dipped for 2-3 seconds in the Agrobacterium solution and afterwards put into solid medium for co-cultivation (co-cultivation medium supplemented with 1.6% Oxoidagar). The co-cultivation lasts 3 days (at 24° C. and ˜50 μMol/m²s light intensity). Afterwards petiols are transferred to co-cultivation medium supplemented with the appropriate selection agent (18 mg/L kanamycin (Duchefa) for plants comprising the nptII marker kanamycin for plants carrying the nptII resistance marker, or 0.3 to 30 mM D-amino acids; as described below) for plants comprising the dual-function marker of the invention) and 300 mg/L Timetin (Duchefa)

Transformed petioles are incubated on the selection medium for four weeks at 24° C. This step is repeated until shoots appear. Shoots are transferred to A6 medium (MS salts (Sigma Aldrich), 20 g/L sucrose, 100 mg/L myo-inositol (Duchefa), 40 mg/L adeninesulfate (Sigma Aldrich), 500 mg/L MES, 0.0025 mg/L BAP (Sigma), 5 g/L oxoidagar (Oxoid GmbH), 150 mg/L timetin (Duchefa), 0.1 mg/L IBA (indol butyric acid, Duchefa); pH 5, 8) supplemented with the appropriate selection agent (18 mg/L kanamycin (Duchefa) for plants comprising the nptII marker kanamycin for plants carrying the nptII resistance marker, or 0.3 to 30 mM D-amino acids; as described below) until they elongated. Elongated shoots are cultivated in A7 medium (A6 medium without BAP) for rooting. Rooted plants are transferred to soil and grown in the greenhouse.

### Example 2

### Selection Analysis

T1 seeds of transgenic Arabidopsis plants were surface-sterilized and sown in Petri plates that were sealed with gas-permeable tape. The growth medium was half strength MS19 with 0.5% (wt/vol) sucrose and 0.8% (wt/vol) agar, plus 3 mM D-alanine, 3 mM D-serine or 50 μg/ml kanamycin as the selective agent. Plants were grown for 5 d after germination with a 16 h photoperiod at 24° C. To evaluate the selection efficiency on different substrates, 2,074, 1,914 and 1,810 T1 seeds were sown on D-alanine-, D-serine- and kanamycin-selective plates, respectively, and the number of surviving seedlings was counted (44, 32 and 43, respectively).

### Example 3

### Toxicity Studies

To evaluate the toxic action of 3-methyl-2-oxopentanoate and 3-methyl-2-oxobutanoate, wild-type plants were sown on two sets of half strength MS agar plates, each containing one of the compounds in a range of concentrations (0.01-10 mM). Plants were slightly affected by 3-methyl-2-oxopentanoate at 0.1 mM, and total growth inhibition was observed at 1 mM. For 3-methyl-2-oxobutanoate, 5 mM was required for complete inhibition. Further, several attempts were made to probe the nature of D-serine's toxicity. In accordance with studies on E. coli, wildtype it was tried to rescue A. thaliana grown on lethal concentrations of D-serine through amendments with five potential inhibitors of D-serine toxicity (L-serine, calcium-pantothenate, β-alanine, leucine and threonine) added both separately and in combinations in a very wide range of concentrations (0.001-50 μg ml-1), without any success.

### Example 4

### Enzyme Assays

Soluble proteins were extracted by shaking 0.1 g samples of plant material that had been finely pulverized in a 1.5 ml Eppendorf tube in 1 ml of 0.1 M potassium phosphate buffer, pH 8. DAAO activity was then assayed as follows. Reaction mixtures were pre-pared containing 2,120 μl of 0.1 M potassium phosphate buffer, pH 8, 80 μl of crude protein extract and 100 μl of 0.3 M D-alanine. The samples were incubated for 2 h at 30° C. The enzyme activity was then assessed, by measuring the increase in absorbance at 220 nm (E=1.090 M⁻¹ cm⁻¹) associated with the conversion of D-alanine to pyruvate, after transferring the test tubes to boiling water for 10 min to stop the reaction. In control reactions, D-alanine was added immediately before boiling. One unit of DAAO activity is defined as the turnover of one micromole of substrate per minute, and activity was expressed per gram plant biomass (fresh weight). The breakdown of D-isoleucine and D-valine in DAAO incubations, and the associated production of 3-methyl-2-oxopentanoate and 3-methyl-2-oxobutanoate, were analyzed by high-performance liquid chromatography. In other respects the reactions were carried out as described above.

### Example 5

### Dual-Function Selection Marker

The qualification of the DAAO enzyme as a dual-function selection marker was demonstrated by testing germinated T1 seeds on different selective media. The T-DNA contained both 35S:dao1 and pNos:nptII, allowing D-amino acid and kanamycin selection to be compared in the same lot of seeds.

T1 seeds were sown on medium containing kanamycin (50 μg/ml), D-alanine (3 mM) or D-serine (3 mM), and the transformation frequencies found on the different selective media were 2.37%, 2.12% and 1.67%, respectively (FIG. 5a-c). D-alanine had no negative effect on the transgenic plants, even at a concentration of 30 mM, but at this concentration, D-serine induced significant growth inhibition. Fewer transgenic plants were found after selection on 3 mM D-serine because the compound slightly inhibited the growth of the transgenic plants at this concentration.

Further studies using lower concentrations corroborated this conclusion, and efficient selection using D-serine was achieved on concentrations lower than 1 mM (FIG. 4a). Progeny from the transgenic lines selected on D-serine and D-alanine were later confirmed to be kanamycin resistant, hence ensuring there would be no wild-type escapes from these lines.

Selection of seedlings on media containing D-alanine or D-serine was very rapid compared to selection on kanamycin. These D-amino acids inhibited growth of wild-type plants immediately after the cotyledons of wild-type plants had emerged. Therefore, transformants could be distinguished from non-transformed plants directly after germination. The difference between wild-type and transgenic plants after D-amino acid selection was unambiguous, with no intermediate phenotypes. In contrast, intermediate phenotypes are common when kanamycin resistance is used as a selection marker (FIG. 5c). Furthermore, wild-type seedlings were found to be sensitive to sprayed applications of D-serine and D-alanine. One-week-old seedlings were effectively killed when sprayed on three consecutive days with either 50 mM D-serine or D-alanine, although the sensitivity of wild-type plants rapidly decreased with age, presumably because as the cuticle and leaves became thicker, uptake by the leaves was reduced. Transgenic seedlings were resistant to foliar application of D-alanine or D-serine, so selection on soil was possible (FIGS. 5d, 11).

Transgenic plants grown under D-alanine and D-serine selection conditions developed normally. Early development of transgenic plants from line 3:7, 10:7 and 13:4 was compared with that of wild-type plants by cultivation on vertical agar plates. No differences in biomass, number of leaves, root length or root architecture were detected for the different sets of plants. Furthermore, soil-cultivated wild-type and transgenic plants (line 10:7) showed no differences in the total number of rosette leaves, number of inflorescences and number of siliqua after 4 weeks of growth.

Also, the phenotypes of 17 individual T1 lines, which were picked for T-DNA segregation, were studied and found indistinguishable from that of wild type when grown on soil. A problem sometimes encountered after selection on antibiotics is the growth lag displayed by transformants. This phenomenon is explained as an inhibitory effect of the antibiotic on the transgenic plants (Lindsey K & Gallois P (1990) J. Exp. Bot 41, 529-536). However, unlike seedlings picked from antibiotic selection plates, transgenic seedlings picked from D-amino acid selection plates and transferred to soil were not hampered in their growth and development, even temporarily. A possible reason for this difference is that the DAAO scavenging of D-amino acids may effectively remove the D-amino acid in the plants. Furthermore, D-alanine and D-serine may merely provide additional growth substrates, because their catabolic products are carbon and nitrogen compounds that are central compounds in plant metabolism. Quantification of dao1 mRNA from six independent D-alanine- and D-serine-resistant lines showed a range of different expression levels (FIG. 5e). These different expression levels were mirrored in a range of different DAAO activities (FIG. 5. In spite of these differences in mRNA levels and enzyme activities, no phenotypic variation associated with the D-serine and D-alanine treatment was found, suggesting that the DAAO marker is effective over a range of expression levels. As described above, D-isoleucine and D-valine were found to inhibit growth of the transgenic plants, but not the wild-type plants.

Therefore, plants containing the construct were tested as described above on two sets of media, one containing D-isoleucine and the other containing D-valine at various concentrations, to assess whether DAAO could also be used as a counter-selection marker. Unambiguous counter-selection selection was achieved when seeds were sown on either D-isoleucine or D-valine at concentrations greater than 10 mM (FIG. 4 c,d).

Thirteen individual lines expressing DAAO were tested for their response to D-isoleucine and all of them were effectively killed, whereas wild-type plants grew well, with no sign of toxicity. Similar results were obtained for D-valine, although this compound was found to have a moderately negative effect on wild-type plants at higher concentrations (FIG. 4 d). The keto acid produced in DAAO catabolism of D-isoleucine is the same as that formed when L-isoleucine is metabolized by the endogenous branched-chain amino acid transaminase [EC: 2.6.1.42], namely 3-methyl-2-oxopentanoate (Kyoto Encyclopedia of Genes and Genomes, metabolic pathway website, http://www.genome.ad.jp/kegg/metabolism.html).

Presumably endogenous transaminase may be specific for the L-enantiomer, so the corresponding D-enantiomer is not metabolized in wildtype plants, but only in plants expressing DAAO. The negative effects of L-isoleucine (but not of the D-form) observed on wildtype plants, supports this speculation. Incubation of cell-free extracts from dao1 transgenic line 10:7 with D-isoleucine and D-valine resulted in 15-fold and 7-fold increases in production of 3-methyl-2-oxopentanoate and 3-methyl-2-oxobutanoate, respectively, compared to extracts of wild-type plants. Further, 3-methyl-2-oxopentanoate and 3-methyl-2-oxobutanoate impaired growth of A. thaliana, corroborating the suggestion that these compounds, or products of their metabolism, are responsible for the negative effects of D-isoleucine and D-valine on the transgenic plants.

The toxicity of some D-amino acids on organisms is not well understood, and has only occasionally been studied in plants (Gamburg K Z & Rekoslavskaya N I (1991) Fiziologiya Rastenii 38, 1236-1246). Apart from A. thaliana, we have also tested the susceptibility of other plant species to D-serine, including poplar, tobacco, barley, maize, tomato and spruce. We found all tested species susceptible to D-serine at concentrations similar to those shown to be toxic for A. thaliana. A proposed mechanism for D-serine toxicity in bacteria is competitive inhibition of a-alanine coupling to pantoic acid, thus inhibiting formation of pantothenic acid (Cosloy S D & McFall E (1973) J. Bacteriol. 114, 685-694). It is possible to alleviate D-serine toxicity in D-serine-sensitive strains of Escherichia coli by providing pantothenic acid or â-alanine in the medium, but D-serine toxicity in A. thaliana could not be mitigated using these compounds. A second putative cause of D-amino acid toxicity is through competitive binding to tRNA. Knockout studies of the gene encoding D-Tyr-tRNATyr deacylase in E coli have shown that the toxicity of D-tyrosine increases in the absence of deacylase activity (Soutourina J et al. (1996) J. Biol. Chem. 274, 19109-19114), indicating that D-amino acids interfere at the tRNA level. Genes similar to that encoding bacterial deacylase have also been identified in A. thaliana (Soutourina J et al. (1996) J. Biol. Chem. 274, 19109-19114), corroborating the possibility that the mode of toxic action of D-amino acids might be through competitive binding to tRNA.

### Example 6

### Constructs Useful for Self-Excising Expression Cassettes Using I-SceI

Two expression constructs are constructed for carrying out the present invention (SEQ ID NO: 15, 16). The backbone of both plasmid constructs (pSUN derivative) contains origins for the propagation in E coli as well as in Agrobacterium and an aadA expression cassette (conferring spectinomycin and streptomycin resistance) to select for transgenic bacteria cells. The sequences for constructing the DNA constructs are amplified incorporating the appropriate restriction sites for subsequent cloning by PCR. Cloning was done by standard methods as described above. The sequence of the constructs is verified by DNA sequence analysis.

### Example 6a

### DAAO Driven by Constitutive Nitrilase Promoter

The first DNA construct (SEQ ID NO: 16) comprises an expression cassette for the D-amino acid oxidase (DAAO) from Rhodotorula gracilis under control of the Arabidopsis thaliana Nitrilase promoter. The DAAO cassette is flanked by a direct repeat of the 35S terminator functioning both as transcription terminator of the DAAO expression cassette and as homology sequences.

Further comprised is a expression cassette for the β-glucuronidase which may function as a substitute for an agronomically valuable trait under control of the Arabidopsis sTPT promoter (i.e. TPT promoter truncated version, WO 03/006660; SEQ ID NO: 27 cited therein), and the CaMV 35S terminator.

### Example 6b

### Self-Excisable DAAO Cassette

The second DNA (SEQ ID NO: 15) comprises an expression cassette for the D-amino acid oxidase (DAAO) from Rhodotorula gracilis under control of the Arabidopsis thaliana Nitrilase promoter. The DNA construct further comprises a Tet on expression system. This allows for induced expression of the I-Sce-I homing endonuclease which is placed under control of a Tet-regulatable promoter. The system further requires expression of the Tet-repressor, which is realized under control of the constitutive ptXA promoter from Pisum sativa.

Both the sequences encoding the DAAO cassette, the I-Sce-I expression cassette, and the rtTA expression cassette (for the reverse tetracycline responsive repressor) are flanked by a direct repeat of the 35S terminator functioning both as transcription terminator of the I-Sce-I expression cassette and as homology sequences.

### Example 7

### Use of the Constructs for the Method of the Invention

### Example 7.1

### Co-Transformation

Arabidopsis thaliana plants are transformed as described above with a mixture of DNA construct I (binary vector SEQ ID NO: 16) and a second binary vector comprising a GFP (green fluorescence protein) expression cassette. In a first selection process transgenic plants are selected comprising both constructs by employing D-alanine mediated selection. 3 mM and 30 mM D-alanine are used.

D-alanine resistant plants comprising the first DNA construct (detectable by GUS staining) also comprising the gfp gene (as assessed by green fluorescence) are isolated and crossed with wild-type plants. Resulting seeds are used for a second counter-selection process, wherein said seeds are germinated on D-isoleucine comprising medium (comprising either 3 mM or 30 mM D-isoleucine). D-isoleucine resistant plants—comprising the gfp gene—can be easily selected.

### Example 7.2

### Marker Excision

Arabidopsis thaliana plants are transformed as described above with a mixture of DNA construct I (binary vector SEQ ID NO: 16). In a first selection process transgenic plants are selected comprising construct I by employing D-alanine mediated selection. 3 mM and 30 mM D-alanine are used.

D-alanine resistant plants comprising the first DNA construct (detectable by GUS staining) are isolated and crossed with a transgenic master plant comprising a transgenic expression cassette for the I-Sce-I homing endonuclease under control of a constitutive promoter (as described in WO 03/004659). Resulting seeds are used for a second counter-selection process, wherein said seeds are germinated on D-isoleucine comprising medium (comprising either 3 mM or 30 mM D-isoleucine). D-isoleucine resistant plants still comprising the GUS-expression cassette can be easily selected.

### Example 7.3

### Use of a Self-Excisable Marker Cassette

Arabidopsis thaliana plants are transformed as described above with a mixture of DNA construct II (binary vector SEQ ID NO: 15). In a first selection process transgenic plants are selected comprising construct II by employing D-alanine mediated selection. 3 mM and 30 mM D-alanine are used.

D-alanine resistant plant cells comprising the DNA construct II are isolated and further cultivated on medium lacking D-alanine. Doxycycline (Sigma; 1 to 5 μg/ml) is added for induction of the marker excision process and cells are incubated for 24 to 48 h on said induction medium. Subsequently cells are further incubated for 3 to 5 days on medium lacking the inducer and D-amino acids (to allow for reduction of DAAO protein levels from prior expression). The resulting cells are used for a second counter-selection process, wherein said cells are further selected on D-isoleucine comprising medium (comprising either 3 mM or 30 mM D-isoleucine). Selected D-isoleucine resistant cells are regenerated into fertile plants and assessed for their transgenic status. By PCR mediated analysis it can be demonstrated that the region flanked by the 35S terminator sequences was accurately excised from the plant genome deleting both the I-SceI expression cassett, the DAAO expression cassette, and the rtTA expression cassette (for the reverse tetracycline responsive repressor)

1. A method for producing a transgenic plant comprising:
i) transforming a plant cell with a first expression cassette comprising a nucleic acid sequence encoding a D-amino acid oxidase operably linked with a promoter allowing expression in plant cells or plants, in combination with at least one second expression cassette suitable for conferring to said plant an agronomically valuable trait, and
ii) providing at least one first compound X, which is phytotoxic against plant cells not functionally expressing said D-amino acid oxidase, wherein said compound X can be metabolized by said D-amino acid oxidase into one or more compound(s) Y which are non-phytotoxic or less phytotoxic than compound X, and
iii) treating said transformed plant cells of step i) with said first compound X in a phytotoxic concentration and selecting plant cells comprising in their genome both said first and said second expression cassette, wherein said first expression cassette is conferring resistance to said transformed plant cells against said compound X by expression of said D-amino acid oxidase, and
iv) providing at least one second compound M, which is non-phytotoxic or moderately phytotoxic against plant cells not functionally expressing said D-amino acid oxidase, wherein said compound M can be metabolized by said D-amino acid oxidase into one or more compound(s) N which are phytotoxic or more phytotoxic than compound M, and
v) breaking the combination between said first expression cassette and said second expression cassette and treating resulting said plant cells with said second compound M in a concentration toxic to plant cells still comprising said first expression cassette, and selecting plant cells comprising said second expression cassette but lacking said first expression cassette.

2. The method of claim 1, wherein said first expression cassette for said D-amino acid oxidase and said second expression cassette for said agronomically valuable trait are
a) both comprised in one DNA construct and combination is broken by deletion or excision of said first expression cassette for said D-amino acid oxidase, or
b) are comprised on separate DNA constructs which are transformed in combination by co-transformation into said plant cells, and combination is broken by subsequent segregation of the two expression cassettes.

3. The method of claim 1, wherein said method for producing a transgenic plant comprises the steps of:
i) transforming a plant cell with a first DNA construct comprising
a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette, and
ii) providing at least one first compound X, which is phytotoxic against plant cells not functionally expressing said D-amino acid oxidase, wherein said compound X can be metabolized by said D-amino acid oxidase into one or more compound(s) Y which are non-phytotoxic or less phytotoxic than compound X, and
iii) treating said transformed plant cells of step i) with said first compound X in a phytotoxic concentration and selecting plant cells comprising in their genome said first DNA construct, conferring resistance to said transformed plant cells against said compound X by expression of said D-amino acid oxidase, and
iv) providing at least one second compound M, which is non-phytotoxic or moderately phytotoxic against plant cells not functionally expressing said D-amino acid oxidase, wherein said compound M can be metabolized by said D-amino acid oxidase into one or more compound(s) N which are phytotoxic or more phytotoxic than compound M, and
v) inducing deletion of said first expression cassette from the genome of said transformed plant cells and treating said plant cells with said second compound M in a concentration toxic to plant cells still comprising said first expression cassette, thereby selecting plant cells comprising said second expression cassette but lacking said first expression cassette.

4. The method of claim 1 further comprising the step of regeneration of a fertile plant.

5. The method of claim 1, wherein said first compound X comprises a D-amino acid structure selected from the group consisting of D-tryptophane, D-histidine, D-arginine, D-threonine, D-methionine, D-serine, and D-alanine, or derivatives thereof.

6. The method of claim 1, wherein said second compound M comprises a D-amino acid structure selected from the group consisting of D-isoleucine, D-valine, D-asparagine, D-leucine, D-lysine, D-proline, and D-glutamine, or derivatives thereof.

7. The method of claim 1, wherein deletion of said first expression cassette for the D-amino acid oxidase is realized by a method selected from:
a) recombination induced by a sequence specific recombinase, wherein said first expression cassette is flanked by corresponding recombination sites in a way that recombination between said flanking recombination sites results in deletion of the sequences in-between from the genome, or
b) homologous recombination between homology sequences A and A′ flanking said first expression cassette, induced by a sequence-specific double-strand break caused by a sequence specific endonuclease, wherein said homology sequences A and A′ have sufficient length and homology in order to ensure homologous recombination between A and A′, and having an orientation which—upon recombination between A and A′—will lead to excision of said first expression cassette from the genome of said plant.

8. The method of claim 7, wherein the recombinase or sequence-specific endonuclease, respectively, is expressed or combined with its corresponding recombination or recognition site, respectively, by a method selected from the group consisting of:
a) incorporation of a second expression cassette for expression of the recombinase or sequence-specific endonuclease operably linked to a plant promoter into said DNA construct, together with said first expression cassette flanked by said sequences which allow for specific deletion,
b) incorporation of a second expression cassette for expression of the recombinase or sequence-specific endonuclease operably linked to a plant promoter into the plant cells or plants used as target material for the transformation thereby generating master cell lines or cells,
c) incorporation of a second expression cassette for expression of the recombinase or sequence-specific endonuclease operably linked to a plant promoter into a separate DNA construct, which is transformed by way of co-transformation with said first DNA construct into said plant cells, and
d) incorporation of a second expression cassette for expression of the recombinase or sequence-specific endonuclease operably linked to a plant promoter into the plant cells or plants which are subsequently crossed with plants comprising the DNA construct of the invention.

9. The method of claim 7, wherein deletion of said first expression cassette for the D-amino acid oxidase is induced or activated by inducing expression and/or activity of said sequence-specific recombinase or endonuclease by a method selected from the group consisting of
a) inducible expression by operably linking the sequence encoding said recombinase or endonuclease to an inducible promoter, and
b) inducible activation, by employing a modified recombinase or endonuclease comprising a ligand-binding-domain, wherein activity of said modified recombinase or endonuclease can by modified by treatment of a compound having binding activity to said ligand-binding-domain.

10. The method of claim 2, wherein the DNA construct comprises
a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette
and the resulting plant cell or plant is selection marker free.

11. A DNA construct suitable for the method of claim 1, comprising
a) a first expression cassette comprising a nucleic acid sequence encoding a D-amino acid oxidase operably linked with a promoter allowing expression in plant cells or plants, wherein said first expression cassette is flanked by sequences which allow for specific deletion of said first expression cassette, and
b) at least one second expression cassette suitable for conferring to said plant an agronomically valuable trait, wherein said second expression cassette is not localized between said sequences which allow for specific deletion of said first expression cassette.

12. The DNA construct of claim 11, wherein said D-amino acid oxidase expressed from said first expression cassette has metabolizing activity against at least one D-amino acid and comprises the following consensus sequence:
[LIVM]-[LIVM]-H*-[NHA]-Y-G-x-[GSA]-[GSA]-x-G-X5-G-x-A
wherein amino acid residues given in brackets represent alternative residues for the respective position, x represents any amino acid residue, and indices numbers indicate the respective number of consecutive amino acid residues.

13. The DNA construct of claim 11, wherein said D-amino acid oxidase has enzymatic activity against at least one of the amino acids selected from the group consisting of D-alanine, D-serine, D-isoleucine, D-valine, and derivatives thereof.

14. The DNA construct of claim 11 wherein said D-amino acid oxidase is described by a sequence of the group consisting of sequences described by GenBank or SwisProt Acc. No. JX0152, O01739, O33145, O35078, O45307, P00371, P14920, P18894, P22942, P24552, P31228, P80324, Q19564, Q28382, Q7PWX4, Q7PWY8, Q7Q7G4, Q7SFW4, Q7Z312, Q82MI8, Q86JV2, Q8N552, Q8P4M9, Q8PG95, Q8R2R2, Q8SZN5, Q8VCW7, Q921M5, Q922Z0, Q95XG9, Q99042, Q99489, Q9C1L2, Q9JXF8, Q9V5P1, Q9VM80, Q9X7P6, Q9Y7N4, Q9Z1M5, Q9Z302, and U60066.

15. The DNA construct of claim 11 wherein said D-amino acid oxidase is selected from the group of amino acid sequences consisting of
a) sequences described by SEQ ID NO: 2, 4, 6, 8, 10, 12, and 14,
b) sequences having a sequence homology of at least 40% with a sequence as described by SEQ ID NO: 2, 4, 6, 8, 10, 12, and 14, and
c) sequences hybridizing under low or high stringency conditions with a sequence as described by SEQ ID NO: 2, 4, 6, 8, 10, 12, and 14.

16. The DNA construct of claim 11, wherein said sequences which allow for specific deletion of said first expression cassette are selected from the group of sequences consisting of
a) recombination sites for a sequences-specific recombinase arranged in a way that recombination between said flanking recombination sites results in deletion of the sequences in-between from the genome, and
b) homology sequences A and A′ having a sufficient length and homology in order to ensure homologous recombination between A and A′, and having an orientation which—upon recombination between A and A′—will result in deletion of the sequences in-between from the genome.

17. The DNA construct of claim 16, wherein said recombination sites correspond to a recombinase selected from the group consisting of a cre recombinase, a FLP recombinase, a Gin recombinase, a Pin recombinase, and a R recombinase.

18. The DNA construct of claim 16, wherein said DNA construct comprises a recognition site of at least 10 base pairs for a sequence specific endonuclease between said homology sequences A and A′.

19. The DNA construct of claim 18, wherein said recognition site corresponds to a sequence-specific endonuclease selected from the group consisting of homing endonucleases I-SceI, I-CpaI, I-CpaII, I-CreI, and I-ChuI and chimeras thereof with ligand-binding domains.

20. The DNA construct of claim 16, wherein said DNA construct further comprises a expression cassette for the sequence specific endonuclease or recombinase suitable for mediating deletion of the first expression cassette for the D-amino acid oxidase.

21. The DNA construct of claim 20, wherein expression and/or activity of said sequence-specific recombinase or endonuclease can be induced and/or activated by a method selected from the group consisting of
a) inducible expression by operably linking the sequence encoding said recombinase or endonuclease to an inducible promoter, and
b) inducible activation, by employing a modified recombinase or endonuclease comprising a ligand-binding-domain, wherein activity of said modified recombinase or endonuclease can by modified by treatment of a compound having binding activity to said ligand-binding-domain.

22. A transgenic vector comprising the DNA construct of claim 11.

23. A transgenic cell comprising the DNA construct of claim 11 or a vector comprising said construct.

24. The transgenic cell of claim 23, wherein said cell is a plant cell.

25. A transgenic, non-human organism comprising the DNA construct of claim 11, a vector comprising said construct, or a transgenic cell comprising said construct or vector.

26. The transgenic, non-human organism of claim 25 wherein said organism is a plant.

27. The method of claim 3 further comprising the step of regeneration of a fertile plant.

28. The method of claim 3, wherein said first compound X comprises a D-amino acid structure selected from the group consisting of D-tryptophane, D-histidine, D-arginine, D-threonine, D-methionine, D-serine, and D-alanine, or derivatives thereof.

29. The method of claim 3, wherein said second compound M comprises a D-amino acid structure selected from the group consisting of D-isoleucine, D-valine, D-asparagine, D-leucine, D-lysine, D-proline, and D-glutamine, or derivatives thereof.
